SEQUENCE LISTING (4 sequences)

SEQ ID NO: 1 - DNA, 1488 bp, Human
atggcggaggaaggagccgtggccgtctgcgtgcgagtgcggccgctgaacagcagagaagaatcacttggagaaactgcccaagtttactggaaaactgacaataatgtcatttatcaagttgatggaagtaaatccttcaattttgatcgtgtctttcatggtaatgaaactaccaaaaatgtgtatgaagaaatagcagcaccaatcatcgattctgccatacaaggctacaatggtactatatttgcctatggacagactgcttcaggaaaaacatataccatgatgggttcagaagatcatttgggagttatacccagggcaattcatgacattttccaaaaaattaagaagtttcctgatagggaatttctcttacgtgtatcttacatggaaatatacaatgaaaccattacagatttactctgtggcactcaaaaaatgaaacctttaattattcgagaagatgtcaataggaatgtgtatgttgctgatctcacagaagaagttgtatatacatcagaaatggctttgaaatggattacaaagggagaaaagagcaggcattatggagaaacaaaaatgaatcaaagaagcagtcgttctcataccatctttaggatgattttggaaagcagagagaagggtgaaccttctaattgtgaaggatctgttaaggtatcccatttgaatttggttgatcttgcaggcagtgaaagagctgctcaaacaggcgctgcaggtgtgcggctcaaggaaggctgtaatataaatcgaagcttatttattttgggacaagtgatcaagaaacttagtgatggacaagttggtggtttcataaattatcgagatagcaagttaacacgaattctccagaattccttgggaggaaatgcaaagacacgtattatctgcacaattactccagtatcttttgatgaaacacttactgctctccagtttgccagtactgctaaatatatgaagaatactccttatgttaatgaggtatcaactgatgaagctctcctgaaaaggtatagaaaagaaataatggatcttaaaaaacaattagaggaggtttctttagagacgcgggctcaggcaatggaaaaagaccaattggcccaacttttggaagaaaaagatttgcttcagaaagtacagaatgagaaaattgaaaacttaacacggatgctggtgacctcttcttccctcacgttgcaacaggaattaaaggctaaaagaaaacgaagagttacttggtgccttggcaaaattaacaaaatgaagaactcaaactatgcagatcaatttaatataccaacaaatataacaacaaaaacacataagctttctataaatttattacgagaaattgatgaatctgtcggtaccatatgggaattcgaagcttacgtagaacaaaaactcatctcagaagaggatctgaatagcgccgtcgaccatcatcatcatcatcat

SEQ ID NO: 2 - protein, 496 aa, Human
MAEEGAVAVCVRVRPLNSREESLGETAQVYWKTDNNVIYQVDGSKSFNFDRVFHGNETTKNVYEEIAAPIIDSAIQGYNGTIFAYGQTASGKTYTMMGSEDHLGVIPRAIHDIFQKIKKFPDREFLLRVSYMEIYNETITDLLCGTQKMKPLIIREDVNRNVYVADLTEEVVYTSEMALKWITKGEKSRHYGETKMNQRSSRSHTIFRMILESREKGEPSNCEGSVKVSHLNLVDLAGSERAAQTGAAGVRLKEGCNINRSLFILGQVIKKLSDGQVGGFINYRDSKLTRILQNSLGGNAKTRIICTITPVSFDETLTALQFASTAKYMKNTPYVNEVSTDEALLKRYRKEIMDLKKQLEEVSLETRAQAMEKDQLAQLLEEKDLLQKVQNEKIENLTRMLVTSSSLTLQQELKAKRKRRVTWCLGKINKMKNSNYADQFNIPTNITTKTHKLSINLLREIDESVGTIWEFEAYVEQKLISEEDLNSAVDHHHHHH

SEQ ID NO: 3 - DNA, 1065 bp, Human
atggcggaggaaggagccgtggccgtctgcgtgcgagtgcggccgctgaacagcagagaagaatcacttggagaaactgcccaagtttactggaaaactgacaataatgtcatttatcaagttgatggaagtaaatccttcaattttgatcgtgtctttcatggtaatgaaactaccaaaaatgtgtatgaagaaatagcagcaccaatcatcgattctgccatacaaggctacaatggtactatatttgcctatggacagactgcttcaggaaaaacatataccatgatgggttcagaagatcatttgggagttatacccagggcaattcatgacattttccaaaaaattaagaagtttcctgatagggaatttctcttacgtgtatcttacatggaaatatacaatgaaaccattacagatttactctgtggcactcaaaaaatgaaacctttaattattcgagaagatgtcaataggaatgtgtatgttgctgatctcacagaagaagttgtatatacatcagaaatggctttgaaatggattacaaagggagaaaagagcaggcattatggagaaacaaaaatgaatcaaagaagcagtcgttctcataccatctttaggatgattttggaaagcagagagaagggtgaaccttctaattgtgaaggatctgttaaggtatcccatttgaatttggttgatcttgcaggcagtgaaagagctgctcaaacaggcgctgcaggtgtgcggctcaaggaaggctgtaatataaatcgaagcttatttattttgggacaagtgatcaagaaacttagtgatggacaagttggtggtttcataaattatcgagatagcaagttaacacgaattctccagaattccttgggaggaaatgcaaagacacgtattatctgcacaattactccagtatcttttgatgaaacacttactgctctccagtttgccagtactgctaaatatatgaagaatactccttatgttaatgaggtatcaactgtcgacaagcttgcggccgcactcgagcaccaccaccaccaccac

SEQ ID NO: 4 - protein, 355 aa, Human
MAEEGAVAVCVRVRPLNSREESLGETAQVYWKTDNNVIYQVDGSKSFNFDRVFHGNETTKNVYEEIAAPIIDSAIQGYNGTIFAYGQTASGKTYTMMGSEDHLGVIPRAIHDIFQKIKKFPDREFLLRVSYMEIYNETITDLLCGTQKMKPLIIREDVNRNVYVADLTEEVVYTSEMALKWITKGEKSRHYGETKMNQRSSRSHTIFRMILESREKGEPSNCEGSVKVSHLNLVDLAGSERAAQTGAAGVRLKEGCNINRSLFILGQVIKKLSDGQVGGFINYRDSKLTRILQNSLGGNAKTRIICTITPVSFDETLTALQFASTAKYMKNTPYVNEVSTVDKLAAALEHHHHHH

(19) United States
(12) Patent Application Publication
(10) Pub. No.: US 2005/0164201 A1
(43) Pub. Date: Jul. 28, 2005

(54) Isolated nucleic acid molecule encoding a novel centromere-associated motor protein, and uses therof

(76) Inventors: Diane Marie Harvey, Lawrenceville, NJ (US); Yi Yang, Dresher, PA (US); Nancy E. Kohl, Wellesley, PA (US)

Correspondence Address: MERCK AND CO., INC, P O BOX 2000, RAHWAY, NJ, 07065-0907, US

(21) Appl. No.: 10/517,510

(22) PCT Filed: Jun. 9, 2003
(86) PCT No.: PCT/US03/18203

Related U.S. Application Data
(60) Provisional application No. 60/387,403, filed on Jun. 10, 2002.

(51) Int. Cl. C12Q 1/68; C07H 21/04; C12N 9/00

(52) U.S. Cl. 435/6; 435/69.1; 435/183; 435/320.1; 435/325; 530/388.26; 536/23.2

(57) ABSTRACT

An isolated nucleic acid is provided which encodes a novel centromere-associated motor protein, HsCENP-E. Also provided are the purified polypeptide encoded by the nucleic acid sequence, and antibodies immunologically specific for the polypeptide. These biological molecules are useful as markers of cellular proliferation, particularly for the identification of cells in the G2 and M phases of the cell cycle. Methods are provided for using the nucleic acid, protein and antibodies for assessing cellular proliferation in biological fluids and tissue samples, and for detecting the presence of autoantibodies to the protein.

### CROSS-REFERENCE TO RELATED APPLICATIONS

This application claims the benefit of U.S. Provisional Application No. 60/387,403, filed Jun. 10, 2002, the contents of which are incorporated herein by reference in their entirety.

### STATEMENT REGARDING FEDERALLY-SPONSORED R&D

Not applicable.

### REFERENCE TO MICROFICHE APPENDIX

Not applicable.

### FIELD OF THE INVENTION

This invention relates generally to genetic engineering involving recombinant DNA technology, and particularly to the identification of nucleic acid molecules encoding a novel centromere-associated motor protein, its gene product and to the use of these sequences in the diagnosis, treatment, and prevention of cancer, and neurological disorders.

### BACKGROUND OF THE INVENTION

Translocation of components within the cell is critical for maintaining cell structure and function. Cellular components such as proteins and membrane-bound organelles are transported along well-defined routes to specific subcellular compartments. Intracellular transport mechanisms utilize microtubules, which are filamentous polymers that serve as tracks for directing the movement of molecules. Molecular transport is driven by the microtubule-based motor proteins, kinesin and dynein. These proteins use the energy derived from ATP hydrolysis to power their movement unidirectionally along microtubules and to transport molecular cargo to specific destinations.

During mitosis, mammalian cells undertake a complex series of steps that ultimately result in the segregation of chromosomes. Mitosis requires dynamic attachment of chromosomes to spindle microtubules. Segregation of genetic material during mitosis is mediated by the microtubules of the mitotic spindle (see, e.g., McIntosh, in Microtubules, pp. 413-434 (Hyams & Lloyd, eds., 1994). Interaction of chromosomes with spindle microtubules is mediated by kinetochores, specialized microtubule attachment structures located at the centromeric region of each sister chromatid (reviewed in Rieder, 1982; Mitchison, 1988.)

The mitotic spindle is a self-organizing structure that is constructed primarily from microtubules. The mitotic spindle undergoes a remarkable series of transitions in response to cell cycle control signals. At each mitotic cell division, the spindle assembles, it forms attachments to each chromosome, it orients itself properly within the cell, and then, with extraordinarily high fidelity, it carries out chromosome segregation. Then it disassembles.

Proper spindle assembly and function involves coordination of many events and processes including modulation of microtubule dynamics and creation of at least three distinct microtubule populations (kinetochore, polar, and astral microtubules). In addition, connections must be established between different spindle microtubule subpopulations, between spindle microtubules and chromosomes, between spindle microtubules and microtubule-associated proteins and motor proteins, and between spindle microtubules and the cell cortex (reviewed by Waters and Salmon, 1997). Proper spindle assembly is monitored by a cellular surveillance system which activates a mitotic checkpoint if the spindle is not assembled correctly (reviewed by Hardwick, 1998; Rudner and Murray, 1996). Once the spindle is assembled, a carefully orchestrated set of molecular events results in chromosome to pole movement (anaphase A) and separation of spindle poles (anaphase B).

Spindle microtubules have a defined polarity, with their slow-growing minus ends anchored at or near the spindle pole, and their dynamic, fast-growing plus ends interacting with chromosomes and with microtubules emanating from the opposite pole (McIntosh and Euteneur, 1984). The two predominant and opposing forces currently thought to be responsible for chromosome movement during congression are an antipoleward polar ejection force associated with regions of high microtubule density near the spindle poles and forces generated directly at the kinetochore by microtubule-dependent motors. Studies in vitro have demonstrated the presence of both plus end- and minus end-directed microtubule motor activities on prometaphase kinetochores (Mitchison and Kirschner, 1985; Hyman and Mitchison, 1991).

During prometaphase, kinetochores capture and stabilize dynamically unstable microtubules growing from the poles (Nicklas and Kubai, 1985; Mitchison et al., 1986). Once attached to microtubules, kinetochores and chromosomes exhibit oscillatory movements switching between poleward and antipoleward movement (Roos, 1976; Bajer, 1982; Reider, et al., 1986; Skibbens et al., 1993; Khodjakov and Rieder, 1996; Waters et al., 1996) and culminating in alignment at the metaphase plate. These movements are collectively referred to as congression, and are thought to be, at least in part, a consequence of forces generated by microtubule motors localized to the kinetochore (Rieder and Salmon, 1994).

Kinetochores are capable of at least three functions in the cell: the attachment of the chromosome to the mitotic spindle through interactions with microtubules, the mediation of mitotic chromosome movements, and the maintenance of a mitotic checkpoint.

The identification and molecular cloning of many proteins of the centromere-kinetochore complex, using autoimmune sera and biochemical fractionation, has provided the necessary reagents to investigate the biochemical structure and function of the complex. The collection of centromere-associated proteins of which six (CENP-A to CENP-F) are currently known can effectively be grouped into two classes, based on their distribution during various times of the cell cycle.

One class, the DNA- or chromatin-binding proteins are constitutive centromere proteins, inasmuch as they can be detected throughout interphase, at discrete loci within the nucleus (presumably centromere chromatin), or localized within the centromere-kinetochore complex during mitosis.

In mammals, four constitutive centromere-binding proteins, CENP-A, CENP-B, CENP-C, and CENP-D, have been characterized to varying extents and implicated to have possible direct roles in centromere function.

CENP-A, a protein localized to the outer kinetochore domain, is a centromere-specific core histone that shows sequence homology to the histone H₃ protein and may serve to differentiate the centromere from the rest of the chromosome at the most fundamental level of chromatin structure—the nucleosome (Sullivan et a., 1994).

CENP-B, a protein which associates with the centromeric heterochromatin through its binding to the CENP-B box motif found in primate α-satellite and mouse minor satellite DNA, probably has a role in packaging centromeric heterochromatic DNA—a role which, however, may not be indispensable since the protein is undetectable on the Y chromosome (Pluta et al., 1990) and is found on the inactive centromeres of dicentric chromosomes (Earnshaw et al., 1989).

CENP-C has been shown to be located at the inner kinetochore plate and is postulated to have an essential although yet undetermined centromere function, as seen, for example, from inhibition of mitotic progression following microinjection of anti-CENP-C antibodies into cells (Bernat et al., 1990; Tomkiel et al., 1994) and from its association with the active but not the inactive centromeres of dicentric chromosomes (Earnshaw et al., 1989; Page et al., 1995; Sullivan and Schwartz 1995).

Finally, CENP-D (or RCC1) is a guanine exchange factor that appears to have a general cellular role that is neither specific nor clear for the centromere (Kingwell and Rattner 1987; Bischoff et al., 1990; Dasso, 1993).

The other class, comprises well characterized proteins such as INCENP, HsCENP-E, CENP-F and CENP-G, which belong to the facultative family of centromere-kinetochore proteins, because of the transient nature of their association with the kinetochore complex. (reviewed by Earnshaw and Mackay, 1994, and Pluta et al., 1995). These passenger proteins, whose appearance at the centromere is transient and tightly regulated by the cell cycle, provide vital functions that include motor movement of chromosomes, modulation of spindle dynamics, nuclear organization, intracellular bridge structure and function, sister chromatid cohesion and release, and cytokinesis.

A human CENP-E gene was first characterized in 1991 and found to span a length of 8371 bases, the open reading frame encodes 2663 amino acids, and the respective protein has a molecular weight of 312 kDa. It is a member of the kinesin superfamily of microtubule motor proteins that is an integral component of kinetochore corona fibers that link centromeres to spindle microtubules (Yen et al., 1992; Yao et al., 1997).

Molecular characterization of the CENP-E molecule shows it to have a tri-partite structure comprised of amino- and carboxy-terminal globular domains separated by a 1,500-residue α-helical domain that is predicted to form coiled-coils. The N-terminal region shares strong sequence homology with the microtubule motor domain of kinesin and kinesin-like proteins (KLPs) (Goldstein, 1993) and binds to microtubules in an ATP-sensitive manner (Liao et al., 1994).

It is a kinetochore motor, which accumulates transiently at kinetochores in the G2 phase of the cell cycle before mitosis takes place that appears to modulate chromosome movement and spindle elongation, and is degraded at the end of mitosis. Cells in G1 and early S phases have little detectable CENP-E, but levels of the protein increase sharply during late S and G2/M. CENP-E associates with kinetochores during congression, relocates to spindle midzones at anaphase, and is discarded or degraded at the end of cell division. CENP-E is believed to serve as an organizing center, facilitating microtubule-kinetochore interaction. Consistently, inhibition of the CENP-E protein by specific antibodies causes cell cycle arrest at metaphase. In addition, it has been implicated is regulating microtubule formation and the consecutive movement of chromosomes during mitosis, and therefore it like other kinesin-like proteins, is crucial for cell division.

More, recent data suggest that CENP-E participates actively in chromosome migration. Microinjection of a CENP-E monoclonal antibody during prometaphase significantly delayed the onset of anaphase (Yen et al., 1991). More recently, some CENP-E antibodies were shown to inhibit poleward chromosome migration driven by microtubule depolymerization in an in vitro assay (Lombillo et al., 1995), while antibodies to, or u.v. induced cleavage of, cytoplasmic dynein (another motor suspected to play a role in anaphase chromosome movement; Steuer et al., 1990; Pfarr et al., 1990) had no effect on this chromosome movement in vitro. Indeed, microinjection of antibody to CENP-E has been shown to block meiotic progression into anaphase I presumably by disrupting the function of CENP-E and/or adjacent components at meiotic kinetochores. [Duesbery N S et al., “CENP-E is an essential kinetochore motor in maturing oocytes and is masked during mos-dependent, cell cycle arrest at metaphase II”, Proc. Natl. Acad. Sci. (USA), 94: 9165-70 (1997).]

As well, data show that depletion of CENP-E from mammalian kinetochores leads to mitotic arrest with a mixture of aligned and unaligned chromosomes. Equally telling is the observation that depletion of CENP-E from kinetochores via antibody microinjection reduces kinetochore microtubule binding by 23% at aligned chromosomes, and severely reduces microtubule binding at unaligned chromosomes. In fact, disruption of CENP-E function also reduces tension across the centromere, increases the incidence of spindle pole fragmentation, and results in monooriented chromosomes approaching abnormally close to the spindle pole.

Likewise, Kullmann et al., “Kinesin-like protein CENP-E is upregulated in rheumatoid synovial fibroblasts” Arthritis Res, 1(1): 71-80 (1999) have demonstrated an up regulation of CENP-E gene expression in Rheumatoid arthritis. Indeed, their study supports the hypothesis that CENP-E, presumably independently from medication, may not only be upregulated, but also be involved in RA pathophysiology.

As indicated above, the mitotic spindle has been the subject of considerable research. The study of mitotic spindle proteins has yielded anti-mitotic compounds with important applications in cancer chemotherapy, and therapeutic agents targeted against fungal pathogens.

Indeed, chemotherapeutics, such as taxol and the vinca alkaloids, have been shown to perturb kinetochore-microtubule attachment and disruption of chromosome segregation, which, in turn, activates a check point pathway that delays cell cycle progression and induces programmed cell death (P. K. Sorger et al. (1997) Curr. Opin. Cell. Biol. 9(6): 807-14; C. M. Ireland et al. (1995) Biochem. Pharmacol. 49(10): 1491-99). As well, Taxol has been demonstrated to induce tubulin polymerization and mitotic arrest, which is followed by apoptosis.

Thus, the demonstrated effectiveness of these anti-mitotic compounds in important medical applications demonstrates the desirability of identifying and characterizing anti-mitotic compound development candidates.

Consequently, a prominent candidate for powering one or more aspects of chromosome movement in mitosis, e.g., inducing cell cycle arrest, is the herein disclosed novel human CENP-E protein.

Importantly, the discovery of a new kinesin-like motor protein and the polynucleotide(s) encoding it satisfies a need in the art by providing new compositions which are useful in the diagnosis, prevention, and treatment of cancer, and other CENP-E mediated disorders.

### SUMMARY OF THE INVENTION

The present invention is based on the discovery of a novel nucleic acid molecule which encodes an essential human centromere-kinetochore protein that has been implicated in regulating microtubule formation and the movement of chromosomes during mitosis. The gene products of the nucleic acid molecule (s) disclosed herein is referred to herein as a centromere-associated motor protein—to wit HsCENP-E. The HsCENP-E molecules of the present invention are useful as modulating agents to regulate a variety of cellular processes, e.g., cell cycle. The protein(s) encoded by the nucleic acid molecule disclosed herein modulates chromosome movement and spindle elongation. As such, the nucleic acid molecules and proteins identified and characterized by the present invention are useful as development candidates for cancer chemotherapeutic agents, anti-fungal compounds, and other anti-mitotic agents.

Accordingly, in one aspect, this invention provides isolated nucleic acid molecules encoding HSCENP-E proteins, as well as nucleic acid fragments suitable as primers or hybridization probes for the detection of HSCENP-E-encoding nucleic acids.

In one embodiment, a HSCENP-E encoding nucleic acid molecule of the invention is at least 50%, 55%, 60%, 65%, 70%, 75%, 80%, 85%, 90%, 95%, 98% or more identical to the nucleotide sequence disclosed herein. In preferred embodiments, the nucleic acid molecules are at least 15 (e.g., contiguous) nucleotides in length and hybridize under stringent conditions to the nucleotide sequences disclosed herein.

In another preferred embodiment, the isolated nucleic acid molecule includes the nucleotide sequence shown SEQ ID NO: 1. Splice variants of the nucleic acid molecule disclosed herein are also encompassed by the present invention.

In another embodiment, a nucleic acid molecule includes a nucleotide sequence encoding a protein having an amino acid sequence sufficiently identical to the amino acid sequence of SEQ ID NO:2.

In other preferred embodiments, the nucleic acid molecule encodes a polypeptide comprising the amino acid sequence of SEQ ID NO:2.

Another embodiment of the invention provides an isolated nucleic acid molecule which is antisense to a HsCENP-E nucleic acid molecule, e.g., the coding strand of a HSCENP-E nucleic acid molecule.

Another aspect of the invention provides a vector comprising a HSCENP-E encoding nucleic acid molecule. In certain embodiments, the vector is a recombinant expression vector.

In another embodiment, the invention provides a host cell containing a vector of the invention.

Methods of producing the gene product encoded by the nucleic acid molecule of the invention are also provided.

A substantially pure polypeptide of SEQ ID NO:2 is also contemplated.

The proteins of the present invention including biologically active fragments thereof, can be operatively linked to a non-HsCENP-E protein (e.g., heterologous amino acid sequences) to form fusion proteins.

The invention further features antibodies, such as monoclonal or polyclonal antibodies, that specifically bind proteins of the invention, preferably HsCENP-E proteins.

In addition, the HsCENP-E proteins or biologically active portions thereof can be incorporated into pharmaceutical compositions, which optionally include pharmaceutically acceptable carriers.

In another aspect, the present invention provides a method for detecting the presence of a HsCENP-E nucleic acid molecule, protein or polypeptide in a biological sample by contacting the biological sample with an agent capable of detecting a HsCENP-E nucleic acid molecule, protein or polypeptide such that the presence of a HsCENP-E nucleic acid molecule, protein or polypeptide is detected in the biological sample.

In another aspect, the invention provides a method for modulating HsCENP-E activity comprising contacting a cell capable of expressing HsCENP-E with an agent that modulates HsCENP-E activity such that HsCENP-E activity in the cell is modulated. In one embodiment, the agent inhibits HsCENP-E activity. In another embodiment, the agent stimulates HsCENP-E activity. In one embodiment, the agent is an antibody that specifically binds to a HsCENP-E protein. In another embodiment, the agent modulates expression of HsCENP-E by modulating transcription of a HsCENP-E gene or translation of a HsCENP-E mRNA. In yet another embodiment, the agent is a nucleic acid molecule having a nucleotide sequence that is antisense to the coding strand of a HsCENP-E mRNA or a HsCENP-E gene.

In one embodiment, the methods of the present invention are used to treat a subject having a disorder characterized by aberrant HsCENP-E protein or nucleic acid expression or activity by administering an agent which is a HsCENP-E modulator to the subject. In one embodiment, the HsCENP-E modulator is a HsCENP-E protein. In another embodiment the HsCENP-E modulator is a HsCENP-E nucleic acid molecule. In yet another embodiment, the HsCENP-E modulator is a peptide, peptidomimetic, or other small molecule. In a preferred embodiment, the disorder is a cellular proliferative disorder characterized by aberrant HsCENP-E protein or nucleic acid expression such as cancer, which is characterized by cells which grow and divide inappropriately with CENP-E being an essential protein for mitotic division.

The present invention also provides a diagnostic assay for identifying the presence or absence of a genetic alteration characterized by at least one of (i) aberrant modification or mutation of a gene encoding a HsCENP-E protein; (ii) mis-regulation of the gene; and (iii) aberrant post-translational modification of a HSCENP-E protein, wherein a wild-type form of the gene encodes a protein with a HsCENP-E activity.

In another aspect the invention provides a method for identifying a compound that binds to or modulates the activity of a HsCENP-E protein, by providing an indicator composition comprising a HsCENP-E protein having HsCENP-E activity, contacting the indicator composition with a test compound, and determining the effect of the test compound on HsCENP-E activity in the indicator composition to identify a compound that modulates the activity of a HsCENP-E protein.

An alternative embodiment contemplates a composition useful as a development candidate for an anti-mitotic agent. The development candidate includes an amino acid sequence of SEQ ID NO:2 or a biologically active fragment thereof.

In an additional embodiment, the invention provides an anti-mitotic agent identified by a screening method using one or more proteins essential to mitotic spindle formation/elongation. In preferred embodiments, the one or more proteins include an amino acid sequence of SEQ ID NO:2 or an amino acid sequence coded for by the HsCENP-E gene of SEQ ID NO:1.

In a further aspect, the invention provides a method of disrupting mitotic spindle formation in a cell. The method involves administering to a cell an anti-mitotic agent that disrupts the activity of one or more proteins essential to mitotic spindle formation. The one or more proteins include an amino acid sequence selected from at least one of the amino acid sequences coded for by the HsCENP-E gene of SEQ ID NO:1 and the amino acid sequence of SEQ ID NO:2.

This invention further provides a method for determining the susceptibility of a biological sample to treatment with a mitotic spindle inhibitor comprising the steps of:
a) contacting the cancer cell containing sample with the antibody capable of specifically binding to HsCENP-E protein, under conditions permitting formation of a complex between the antibody and the HsCENP-E protein in the sample; and
b) detecting the complex formed in step a), the presence of the complex indicating that the cancer cell containing sample is susceptible to treatment with a mitotic spindle inhibitor.

This invention also provides a method of determining whether a biological sample suspected of containing cancerous cells is susceptible to treatment with a mitotic spindle inhibitor by detecting the presence of HsCENP-E protein in the sample which comprises:
a) contacting a biological sample suspected of containing cancerous cells with a polynucleotide probe comprising at least 15 contiguous nucleotides derived from SEQ ID NO:1 under conditions permitting the hybridization of the probe to the RNA present in the sample; and
b) detecting the presence of the hybridized probe, a positive detection indicating susceptibility to treatment with a mitotic spindle inhibitor.

This invention further provides a method of suppressing hyper-proliferative cell growth disorder in a subject which comprises administering the nucleic acid molecule encoding a HsCENP-E protein to the subject in an amount effective to decrease expression of the HsCENP-E protein.

This invention also provides a pharmaceutical composition comprising an amount of the antisense oligonucleotide having a sequence capable of specifically hybridizing to mRNA encoding for a HsCENP-E protein so as to prevent translation of the mRNA, which is capable of passing through a cell membrane and effective to inhibit the expression of HsCENP-E and a suitable pharmaceutically acceptable carrier.

This invention also provides a nucleic acid molecule reagent capable of detecting the HsCENP-E gene or gene product.

This invention also provides a method for in situ identification of HsCENP-E mediated pathologies which may be susceptible to treatment with mitotic spindle inhibitors by detecting the presence of nucleic acid molecule encoding HsCENP-E in a cancerous cell which method comprises contacting the cell with a suitably labeled nucleic acid molecule reagent capable of detecting the HsCENP-E gene or gene product.

Yet another aspect of the present invention is a method of inhibiting disease-associated proliferation of cells comprising the step of contacting the cells with a purified polynucleotide comprising the nucleotide sequence as set forth in SEQ ID NO:1 or a fragment of from 12 to 100 nucleotides in length derived therefrom, said polynucleotide being substantially complementary to a nucleic acid sequence region of 50 nucleotides present in the mRNA encoding HsCENP-E (but having T substituted for U), wherein said oligonucleotide inhibits proliferation of cells in vivo or in vitro.

A diagnostic method for predicting an oncogenic potential of a sample of cells, comprising:
(a) determining, in the sample, levels of expression of a gene product expressed from a nucleotide sequence which hybridizes with a nucleotide sequence corresponding to SEQ ID NO:1 or its complement.

Also provided is a method for following progress of a therapeutic regime designed to alleviate a condition characterized by abnormal expression of a gene product expressed from the isolated nucleic acid molecule having a sequence of nucleotides as set forth in SEQ ID NO:1 comprising:
(a) assaying a sample from a subject to determine level of a parameter selected from the group consisting of (i) a polypeptide encoded by a the nucleotide sequence of SEQ D NO:1 and (ii) a polynucleotide encoding the amino acid sequence of SEQ ID NO:2, at a first time point;
(b) assaying level of the parameter selected in (a) at a second time point and
(c) comparing said level at said second time point to the level determined in (a) as a determination of effect of said therapeutic regimé.

An alternative embodiment provides a method for determining regression, progression or onset of a pathological disorder characterized by an aberrant level or activity of a HsCENP-E protein comprising incubating a sample obtained from a patient with said disorder with a complimentary nucleic acid hybridization probe having a sequence of nucleotides that are substantially homologous to those of SEQ ID NO:1 and determining binding between said probe and any complimentary mRNA that may be present in said sample as determinative of the regression, progression or onset of said pathological disorder in said patient.

Alternatively, the probe may encompass an antibody specific for the polypeptide of SEQ ID NO:2 or a biologically/immunologically active fragment thereof. Consequently, the proposed method is drawn to determining regression, comprising: contacting a sample, from a patient with said disorder, with a detectable probe that is specific for the gene product expressed by the isolated nucleic acid molecule of the invention, under conditions favoring formation of a probe/gene product complex, the presence of which is indicative of the regression, progression or onset of said pathological disorder in said patient.

Other uses and objectives of this invention will be apparent to those of ordinary skill in the art in view of the Detailed Description which follows. Such other uses and objectives are deemed to be within the scope of the claims which follow.

### BRIEF DESCRIPTION OF THE DRAWINGS

FIG. 1 (SEQ ID NO:1) discloses a nucleotide sequence of 1395 base pairs that encodes a novel motor domain of human CENP-E protein—referred to as CENP-E 465.

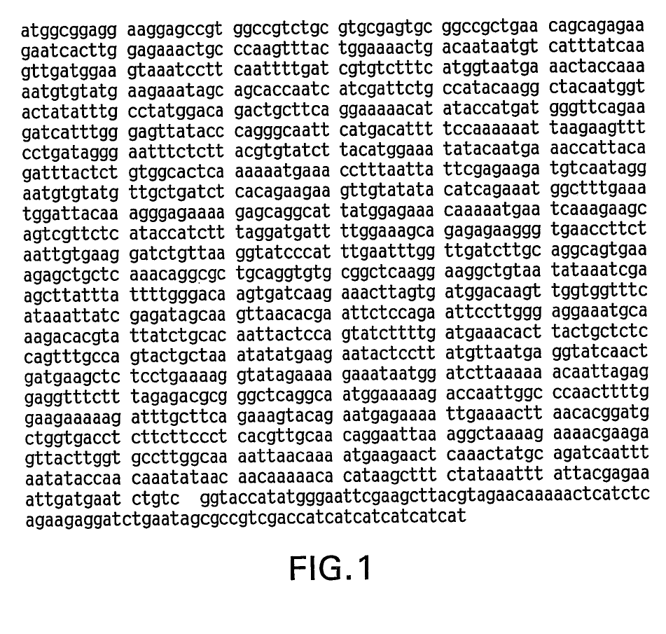

FIG. 2 (SEQ ID NO:2) is the amino acid sequence of the motor protein encoded by the nucleotide sequence as set forth in SEQ ID NO:1-465 amino acids.

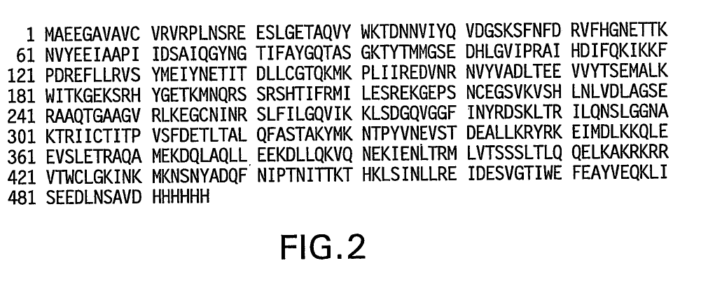

FIG. 3 (SEQ ID NO:3) corresponds to the nucleotide sequence of 1020 base pairs which encodes a novel motor domain of human CENP-E protein of 340 amino acids, also referred to as CENP-E340.

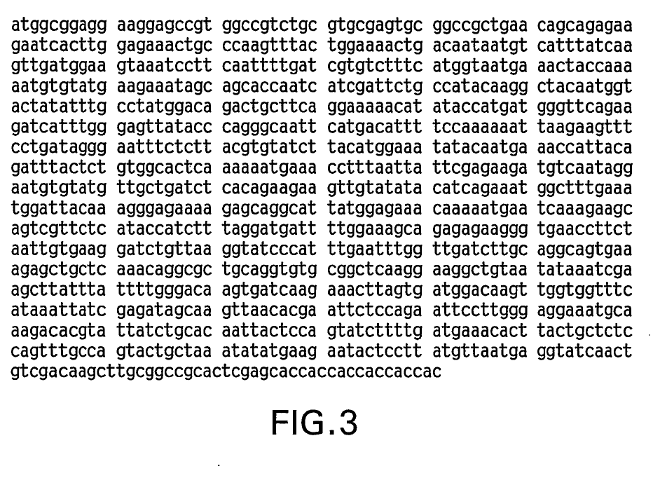

FIG. 4 (SEQ ID NO:4) is the amino acid sequence of the motor protein encoded by the nucleotide sequence as set forth in SEQ ID NO: 3-340 amino acids.

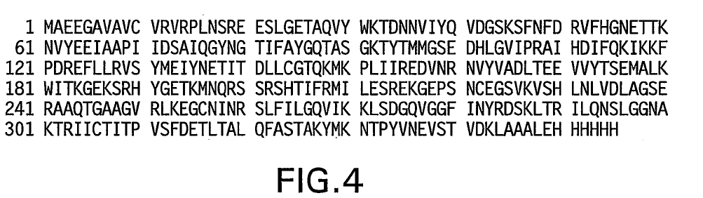

### DETAIL DESCRIPTION OF THE INVENTION

The present invention is based on the discovery of a new human kinesin motor protein, human CENP-E (HsCENP-E), the polynucleotide encoding HsCENP-E, and the use of these compositions for the diagnosis, treatment, or prevention of cancer, neurological disorders, and disorders of vesicular transport. Also provided are modulators of a target protein, e.g., native CENP-E including agents for the treatment of cellular proliferating disorders such as cancer. The agents and compositions provided herein can be used in a variety of applications which include the formulation of sprays, powders, and other compositions. Methods of treating cellular proliferation disorders such as cancer, for treating disorders associated with HsCENP-E activity, and for inhibiting HsCENP-E are also provided.

In particular, the invention is based upon the discovery-cloning and expression, of a novel DNA fragment of about 1395 nucleotides that encodes a novel motor domain of human CENP-E of about 465 amino acids. The encoded protein fragment is functional in that it includes microtubule binding activity as well as an ATP binding site and its attending ATPase activity.

The resulting protein can be distinguished from the prior art CENP-E based upon the presence of alanine at position 300 instead of proline (published sequence). With respect to the novel DNA sequence provide herein, it differs from the published sequence in at least the following positions—
base pair 876 T (published sequence)>C, which corresponds to amino acid 292 of SEQ ID NO: 2,
base pair 898 C (published sequence)>G, which corresponds to amino acid at position 300
base pair 948 T (published sequence)>A, which corresponds to amino acid position 316 relative to the published sequence.

Importantly, unlike the two changes at positions 876 and 948, which do not result in a change in the encoded amino acid of the published sequence, the change at position 898 results in a change in the encoded protein in that proline (published sequence) is changed to Alanine at the same position.

Amino acid at position 292 of the herein disclosed sequence corresponds to position 292 of the published sequence.

Amino acid at position 300 of the herein disclosed sequence corresponds to position 300 of the published sequence.

Amino acid at position 316 of the herein disclosed sequence corresponds to position 316 of the published sequence.

The numbering of the amino acids in the CENP-E protein(s) of the invention correspond to the numbering of the published protein. However, with regards to the cDNA sequence, base pairs 1-1395 disclosed herein correspond to base pairs 91-1485 in the published CENP-E cDNA nucleotide sequence. The nucleotide and amino acid sequence of the prior art CENP-E protein are disclosed in Yen, T. J. et al. (1991) “CENP-E, a novel human centromere-associated protein required for progression from metaphase to anaphase.” EMBO J. 10(5): 1245-1254; and Yen, T. J. et al. (1992) “CENP-E is a putative kinetochore motor that accumulates just before mitosis.” Nature 359(6395): 536-539. The contents of each of the references is incorporated by reference herein in its entirety.

Before the present proteins, nucleotide sequences, and methods are described, it is understood that this invention is not limited to the particular machines, materials and methods described, as these may vary. It is also to be understood that the terminology used herein is for the purpose of describing particular embodiments only, and is not intended to limit the scope of the present invention which will be limited only by the appended claims.

In the description that follows, a number of terms used in the field of recombinant DNA technology are extensively utilized. In order to provide a clearer and consistent understanding of the specification and claims, including the scope to be given such terms, the following definitions are provided.

A “gene” refers to a nucleic acid molecule whose nucleotide sequence codes for a polypeptide molecule. Genes may be uninterrupted sequences of nucleotides or they may include such intervening segments as introns, promoter regions, splicing sites and repetitive sequences. A gene can be either RNA or DNA. A preferred gene is one that encodes the invention protein.

The term “nucleic acid” “nucleic acid molecule” “polynucleotide” “oligonucleotide” or grammatical equivalents thereof is intended for ribonucleic acid (RNA) or deoxyribonucleic acid (DNA), probes, polynucleotides, fragment or portions thereof, and primers. DNA can be either complementary DNA (cDNA) or genomic DNA, e.g. a gene encoding the invention protein.

For a DNA molecule or polynucleotide, a nucleotide sequence refers to a sequence of deoxyribonucleotides, and for an RNA molecule or polynucleotide, the corresponding sequence of ribonucleotides (A, G, C and U), where each thymidine deoxyribonucleotide (T) in the specified deoxyribonucleotide sequence is replaced by the ribonucleotide uridine (U). For instance, reference to an RNA molecule having the sequence of SEQ ID NO:1 set forth using deoxyribonucleotide abbreviations is intended to indicate an RNA molecule having a sequence in which each deoxyribonucleotide A, G or C of SEQ ID NO:1 has been replaced by the corresponding ribonucleotide A, G or C, and each deoxyribonucleotide T has been replaced by a ribonucleotide U.

Unless otherwise indicated, a nucleotide defines a monomeric unit of DNA or RNA consisting of a sugar moiety (pentose), a phosphate group, and a nitrogenous heterocyclic base. The base is linked to the sugar moiety via the glycosidic carbon (1′ carbon of the pentose) and that combination of base and sugar is a nucleoside. When the nucleoside contains a phosphate group bonded to the 3′ or 5′ position of the pentose, it is referred to as a nucleotide. A sequence of operatively linked nucleotides is typically referred to herein as a “base sequence” or “nucleotide sequence”, and their grammatical equivalents, and is represented herein by a formula whose left to right orientation is in the conventional direction of 5′-terminus to 3′-terminus.

Unless otherwise indicated, a particular nucleic acid molecule sequence also implicitly encompasses conservatively modified variants thereof (e.g., degenerate codon substitutions) and complementary sequences and as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic acid molecule Res. 19: 5081 (1991); Ohtsuka et al., J. Biol. Chem. 260.2605-2608 (1985); Cassol et al., 1992; Rossolini et al., Mol. Cell. Probes 8: 91-98 (1994)). The term nucleic acid molecule is used interchangeably with gene, cDNA, and mRNA encoded by a gene.

A “fragment” of a nucleic acid molecule or nucleotide sequence is a portion of the nucleic acid that is less than full-length and comprises at least a minimum length capable of hybridizing specifically with the nucleotide sequence of SEQ ID NO:1 under stringent hybridization conditions. The length of such a fragment is preferably 15-17 nucleotides or more.

“Variant” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCT all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each degenerate codon in a nucleic acid can be modified to yield a functionally identical molecule. Accordingly, each silent variation of a nucleic acid which encodes a polypeptide is implicit in each described sequence. Included within this definition are polymorphisms which may or may not be readily detectable using a particular oligonucleotide probe of the polynucleotide encoding HsCENP-E, and improper or unexpected hybridization to allelic variants, with a locus other than the normal chromosomal locus for the polynucleotide sequence encoding HsCENP-E.

Also included within the definition of target proteins of the present invention are amino acid sequence variants of wild-type target proteins. These variants fall into one or more of three classes: substitutional, insertional or deletional variants. These variants ordinarily are prepared by site specific mutagenesis of nucleotides in the DNA encoding the target protein, using cassette or PCR mutagenesis or other techniques well known in the art, to produce DNA encoding the variant, and thereafter expressing the DNA in recombinant cell culture. Variant target protein fragments having up to about 100-150 amino acid residues may be prepared by in vitro synthesis using established techniques. Amino acid sequence variants are characterized by the predetermined nature of the variation, a feature that sets them apart from naturally occurring allelic or interspecies variation of the target protein amino acid sequence. The variants typically exhibit the same qualitative biological activity as the naturally occurring analogue, although variants can also be selected which have modified characteristics.

Amino acid substitutions are typically of single residues; insertions usually will be on the order of from about 1 to about 20 amino acids, although considerably longer insertions may be tolerated. Deletions range from about 1 to about 20 residues, although in some cases, deletions may be much longer.

Substitutions, deletions, and insertions or any combinations thereof may be used to arrive at a final derivative. Generally, these changes are done on a few amino acids to minimize the alteration of the molecule. However, larger characteristics may be tolerated in certain circumstances.

Individual substitutions, to a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art (Henikoff and Henikoff (Proc. Natl. Acad. Sci. USA 89; 10915-10919 (1992))).

“HsCENP-E” or “CENP-E” are used interchangeably refers to a centromere-associated motor protein having the amino acid sequence as set forth in SEQ ID NO:2 or an allelic or biologically active fragment thereof. “Hs” refers to homo sapien. HsCENP-E is an integral component of the kinetochore structure of the chromosome, which links the chromosome to the spindle microtubules. HsCENP-E has activity such as ATPase activity, microtubule binding activity, and plus end-directed microtubule motor activity.

A “HsCENP-E polynucleotide(s)” refer to polynucleotides (DNA or RNA) containing a nucleotide sequence which encodes a HsCENP-E protein or a biologically active fragment thereof, or a sequence of nucleotides that hybridize under high stringency conditions to the nucleotide sequence disclosed herein. Such a nucleic acid molecule can be characterized in a number of ways, for example—the nucleic acid molecule may encode the amino acid sequence set forth in SEQ ID NO:2, or a nucleotide sequence which has at least 75% identity to a nucleotide sequence encoding the polypeptide of SEQ ID NO:2 or the corresponding fragment thereof, or a nucleotide sequence which has sufficient identity to a nucleotide sequence contained in SEQ ID NO:1 or allelic variants thereof, splice variants thereof and/or their complements.

“Invention nucleic acid(s)” and “nucleic acid molecules” are used interchangeably and refer to the nucleic acid molecule(s) set forth herein.

As used herein, a “splice variant” refers to variant invention protein(s)—encoding nucleic acid(s) produced by differential processing of primary transcript(s) of genomic DNA, resulting in the production of more than one type of mRNA. cDNA derived from differentially processed primary transcript will encode the HsCENP-E protein(s) of the invention that has regions of complete amino acid identity and regions having different amino acid sequences. Thus, the same genomic sequence can lead to the production of multiple, related mRNAs and proteins. Both the resulting mRNAs and proteins are referred to herein as “splice variants”.

As used herein a “polynucleotide probe” is defined as a nucleotide sequence capable of binding to a target nucleic acid molecule of complementary sequence through one or more types of chemical bonds, usually through complementary base pairing, usually through hydrogen bond formation. Presently preferred probe-based screening conditions which allow the identification of sequences having at least 70% homology with the probe, while discriminating against sequences which have a lower degree of homology with the probe. As a result, nucleic acids having substantially the same nucleotide sequence as the sequence of nucleotides set forth in SEQ ID NO:1 are obtained.

“Hybridization” refers to the binding of complementary strands of nucleic acid (i.e., sense:antisense strands or probe:target-DNA) to each other through hydrogen bonds, similar to the bonds that naturally occur in chromosomal DNA. Stringency levels used to hybridize a given probe with target-DNA can be readily varied by those of skill in the art.

The phrase “stringent hybridization conditions” is used herein to refer to conditions under which polynucleic acid hybrids are stable. As known to those of skill in the art, the stability of hybrids is reflected in the melting temperature (Tₘ) of the hybrids. Tₘ can be approximated by the formula:
81.5° C.−16.6(log₁₀[Na⁺])+0.41(% G+C)−600/1,
where 1 is the length of the hybrids in nucleotides. Tₘ decreases approximately 1°-1.5° C. with every 1% decrease in sequence homology. In general, the stability of a hybrid is a function of sodium ion concentration and temperature. Typically, the hybridization reaction is performed under conditions of lower stringency, followed by washes of varying, but higher, stringency. Reference to hybridization stringency relates to such washing conditions.

As used herein, the phrase “moderately stringent hybridization” refers to conditions that permit target-DNA to bind a complementary nucleic acid that has about 60% identity, preferably about 75% identity, more preferably about 85% identity to the target DNA; with greater than about 90% identity to target-DNA being especially preferred. Preferably, moderately stringent conditions are conditions equivalent to hybridization in 50% formamide, 5× Denhart's solution, 5×SSPE, 0.2% SDS at 42° C., followed by washing in 0.2×SSPE, 0.2% SDS, at 65° C. The skilled artisan will recognize how to adjust the temperature, ionic strength, etc. as necessary to accommodate factors such as probe length and the like. (See Sambrook et al., Molecular Cloning: A Laboratory Manual, New York: Cold Spring Harbor Press, 1989).

“High stringency conditions”, as defined herein, may be identified by those that: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5× Denhardt's solution, sonicated salmon sperm DNA (50 Jig/ml), 0.1% SDS, and 10% dextran sulfate at 42° C., with washes at 42° C. in 0.2×SSC (sodium chloride/sodium citrate) and 50% formamide at 55° C., followed by a high-stringency wash consisting of 0.1×SSC containing EDTA at 55° C.

The phrase “low stringency hybridization” refers to conditions equivalent to hybridization in 10% formamide, 5× Denhart's solution, 6×SSPE, 0.2% SDS at 42° C., followed by washing in 1×SSPE, 0.2% SDS, at 50° C.

Denhardt's solution and SSPE (see, e.g., Sambrook, Fritsch, and Maniatis, in: Molecular Cloning, A Laboratory Manual, Cold Spring Harbor Laboratory Press, 1989) are well known to those of skill in the art as are other suitable hybridization buffers. For example, SSPE is pH 7.4 phosphate-buffered 0.18M NaCl. SSPE can be prepared, for example, as a 20× stock solution by dissolving 175.3 g of NaCl, 27.6 g of NaH₂PO₄ and 7.4 g EDTA in 800 ml of water, adjusting the pH to 7.4, and then adding water to 1 liter. Denhardt's solution (see, Denhardt (1966) Biochem. Biophys. Res. Commun. 23: 641) can be prepared, for example, as a 50× stock solution by mixing 5 g Ficoll (Type 400, Pharmacia LKB Biotechnology, Inc., Piscataway N.J.), 5 g of polyvinylpyrrolidone, and 5 g bovine serum albumin (Fraction V; Sigma, St. Louis Mo.), and then adding water to 500 ml and filtering to remove particulate matter.

Preferred nucleic acids encoding the invention polypeptide(s) hybridize under moderately stringent, preferably high stringency, conditions to substantially the entire sequence, or substantial portions (i.e., typically at least 15-30 nucleotides) of the nucleic acid sequence set forth in SEQ ID NO:1 (HsCENP-E).

In defining nucleic acid sequences, all subject nucleic acid sequences capable of encoding “substantially similar amino acid sequences” are considered substantially similar or are considered as comprising substantially identical sequences of nucleotides to the reference nucleic acid sequence, i.e., HsCENP-E encoding sequence—SEQ ID NO:1.

In practice, the term “substantially the same sequence” means that DNA or RNA encoding two proteins hybridize under moderately stringent conditions and encode proteins that have the same sequence of amino acids or have changes in sequence that do not alter their structure or function.

Nucleotide sequence “similarity” is a measure of the degree to which two polynucleotide sequences have identical nucleotide bases at corresponding positions in their sequence when optimally aligned (with appropriate nucleotide insertions or deletions). Sequence similarity or percent similarity can be determined, for example, by comparing sequence information using sequence analysis software such as the GAP computer program, version 6.0, available from the University of Wisconsin Genetics Computer Group (UWGCG). The GAP program utilizes the alignment method of Needleman and Wunsch (J. Mol. Biol. 48: 443, 1970), as revised by Smith and Waterman (Adv. Appl. Math. 2: 482, 1981).

“Identity,” as known in the art, is a relationship between two or more polypeptide sequences or two or more polynucleotide sequences, as the case may be, as determined by comparing the sequences. “Identity” can be readily calculated by known methods, including but not limited to those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New, York, 1991; and Carillo, H., and Lipman, D., SIAM J Applied Math., 48: 1073 (1988). Methods to determine identity are designed to give the largest match between the sequences tested. Moreover, methods to determine identity are codified in publicly available computer programs. Computer program methods to determine identity between two sequences include, but are not limited to, the GCG program package (Devereux, J., et al., Nucleic Acids Research 12(1): 387 (1984)), BLASTP, BLASTN, and FASTA (Atschul, S. F. et al., J. Molec. Biol. 215: 403-410 (1990). The BLAST X program is publicly available from NCBI and other sources (BLAST Manual, Altschul, S., et al., NCBI NLM NIH Bethesda, Md. 20894; Altschul, S., et al., J. Mol. Biol. 215: 403-410 (1990). The well known Smith Waterman algorithm may also be used to determine identity.

Parameters for polypeptide sequence comparison include the following:
1) Algorithm: Needleman and Wunsch, J. Mol. Biol. 48: 443-453 (1970)
Comparison matrix: BLOSSUM62 from Hentikoff and Hentikoff, Proc. Natl. Acad. Sci. USA. 89: 10915-10919 (1992)
Gap Penalty: 12
Gap Length Penalty: 4

A program useful with these parameters is publicly available as the “gap” program from Genetics Computer Group, Madison Wis. The aforementioned parameters are the default parameters for peptide comparisons (along with no penalty for end gaps).

Parameters for polynucleotide comparison include the following:
1) Algorithm: Needleman and Wunsch, J. Mol. Biol. 48: 443-453 (1970)
Comparison matrix: matches=+10, mismatch=0
Gap Penalty: 50
Gap Length Penalty: 3

Available as: The “gap” program from Genetics Computer Group, Madison Wis. These are the default parameters for nucleic acid comparisons.

A preferred meaning for “identity” for polynucleotides and polypeptides, as the case may be, are provided in (1) and (2) below.

(1) Polynucleotide embodiments further include an isolated polynucleotide comprising a polynucleotide sequence having at least a 50, 60, 70, 80, 85, 90, 95, 97 or 100% identity to the reference sequence of SEQ ID NO:1, wherein the polynucleotide sequence may be identical to the reference sequence of SEQ ID NO:1 or may include up to a certain integer number of nucleotide alterations as compared to the reference sequence, wherein the alterations are selected from the group consisting of at least one nucleotide deletion, substitution, including transition and transversion, or insertion, and wherein the alterations may occur at the 5′ or 3′ terminal positions of the reference nucleotide sequence or anywhere between those terminal positions, interspersed either individually among the nucleotides in the reference sequence or in one or more contiguous groups within the reference sequence, and wherein the number of nucleotide alterations is determined by multiplying the total number of nucleotides in SEQ ID NO:1 by the integer defining the percent identity divided by 100 and then subtracting that product from the total number of nucleotides in SEQ ID NO:1, or:
NₙXₙ−(XₙY),
wherein Nₙ is the number of nucleotide alterations, Xₙ is the total number of nucleotides in SEQ ID NO:1, Y is 0.50 for 50%, 0.60 for 60%, 0.70 for 70%, 0.80 for 80%, 0.85 for 85%, 0.90 for 90%, 0.95 for 95%, 0.97 for 97% or 1.00 for 100%, and is the symbol for the multiplication operator, and wherein any non-integer product of Xₙ and Y is rounded down to the nearest integer prior to subtracting it from Xₙ Alterations of a polynucleotide sequence encoding the polypeptide of SEQ ID NO:2 may create nonsense, missense or frameshift mutations in this coding sequence and thereby alter the polypeptide encoded by the polynucleotide following such alterations.

(2) Polypeptide embodiments further include an isolated polypeptide comprising a polypeptide having at least a 50, 60, 70, 80, 85, 90, 95, 97 or 100% identity to a polypeptide reference sequence of SEQ ID NO:2, wherein the polypeptide sequence may be identical to the reference sequence of SEQ ID NO:2 or may include up to a certain integer number of amino acid alterations as compared to the reference sequence, wherein the alterations are selected from the group consisting of at least one amino acid deletion, substitution, including conservative and non-conservative substitution, or insertion, and wherein the alterations may occur at the amino- or carboxy-terminal positions of the reference polypeptide sequence or anywhere between those terminal positions, interspersed either individually among the amino acids in the reference sequence or in one or more contiguous groups within the reference sequence, and wherein the number of amino acid alterations is determined by multiplying the total number of amino acids in SEQ ID NO:2 by the integer defining the percent identity divided by 100 and then subtracting that product from the total number of amino acids in SEQ ID NO:2, or:
Nₐ=Xₐ−(XₐY),
herein Nₐ is the number of amino acid alterations, Xₐ is the total number of amino acids in SEQ ID NO:2, Y is 0.50 for 50%, 0.60 for 60%, 0.70 for 70%, 0.80 for 80%, 0.85 for 85%, 0.90 for 90%, 0.95 for 95%, 0.97 for 97% or 1.00 for 100%, and is the symbol for the multiplication operator, and wherein any non-integer product of Xₐ and Y is rounded down to the nearest integer prior to subtracting it from Xₐ.

For example, a designated amino acid percent identity of 70% refers to sequences or subsequences that have at least about 70% amino acid identity when aligned for maximum correspondence over a comparison window as measured using one of the sequence comparison algorithms disclosed herein and well known to a skilled artisan or by manual alignment and visual inspection. It is recognized, however, that proteins (and DNA or mRNA encoding such proteins) containing less than the above-described level of homology arising as splice variants or that are modified by conservative amino acid substitutions (or substitution of degenerate codons) are contemplated to be within the scope of the present invention.

The present invention also encompasses nucleic acids which differ from the nucleic acids shown in SEQ ID NO:1, but which have the same phenotype. Phenotypically similar nucleic acids are also referred to as “functionally equivalent nucleic acids”.

As used herein, the phrase “functionally equivalent nucleic acids” encompasses nucleic acids characterized by slight and non-consequential sequence variations that will function in substantially the same manner to produce the same protein product(s) as the nucleic acids disclosed herein. These changes include those recognized by those of skill in the art as those that do not substantially alter the tertiary structure of the protein.

By “protein” herein is meant at least two covalently attached amino acids, which includes proteins, polypeptides, oligopeptides, and peptides. Thus “amino acid”, or “peptide residue”, as used herein means both naturally occurring and synthetic amino acids. The side chains may be in either the (R) or the (S) configuration. In the preferred embodiment, the amino acids are in the (S) or L-configuration. If non-naturally occurring side chains are used, non-amino acid substituents may be used, for example to prevent or retard in vivo degradations.

The terms “polypeptide”, “peptide” and “protein” are used interchangeably herein to refer to a polymer of amino acid residues. The terms apply to amino acid polymers in which one or more amino acid residues is an artificial chemical analogue of a corresponding naturally occurring amino acid, as well as to naturally occurring amino acid polymers. A target protein comprises a polypeptide demonstrated to have at least microtubule stimulated ATPase activity and, preferably that also binds to an antibody selectively immunoreactive with HsCENP-E or whose sequence is derived from HsCENP-E by mutagenesis and/or recombination. Amino acids may be referred to herein by either their commonly known one or three letter symbols. Nucleotides, likewise, may be referred to by their commonly accepted single-letter codes. The target protein may also be “altered,” and may contain deletions, insertions, or substitutions of amino acid residues which produce a silent change and result in a functionally equivalent HsCENP-E (e.g., variant HsCENP-E). Deliberate amino acid substitutions may be made on the basis of similarity in polarity, charge, solubility, hydrophobicity, hydrophilicity, and/or the amphipathic nature of the residues, as long as the biological or immunological activity of HsCENP-E is retained. For example, negatively charged amino acids may include aspartic acid and glutamic acid, positively charged amino acids may include lysine and arginine, and amino acids with uncharged polar head groups having similar hydrophilicity values may include leucine, isoleucine, and valine; glycine and alanine; asparagine and glutamine; serine and threonine; and phenylalanine and tyrosine.

The term “amino acid sequence” as used herein refers to an oligopeptide, peptide, polypeptide, or protein sequence, and fragments or portions thereof, and to naturally occurring or synthetic molecules.

A “fragment” or “a biologically active fragment” of the reference protein, e.g., SEQ ID NO:2 is meant to refer to a protein which contains a portion of the complete amino acid sequence of the wild type or reference protein. Such molecules are expected to have, inter alia, similar biological functions/properties equivalent to their wild type homologous polypeptides and polynucleotides. Furthermore, preferred polypeptides and polynucleotides of the present invention have at least one HsCENP-E mediated activity.

“Biologically active” when used in conjunction with either “HsCENP-E” or “isolated HsCENP-E” or a “target protein” refers to a protein that has one or more of kinesin protein's biological activities, including, but not limited to microtubule stimulated ATPase activity, as tested, e.g., in an ATPase assay. Biological activity can also be demonstrated in a microtubule gliding assay or a microtubule binding assay. As used herein, biological activity attending a HsCENP-E protein refers to any activity characteristic of human CENP-E, see supra.

“ATPase activity” refers to ability to hydrolyze ATP. Other activities include polymerization/depolymerization (effects on microtubule dynamics), binding to other proteins of the spindle, binding to proteins involved in cell-cycle control, or serving as a substrate to other enzymes, such as kinases or proteases and specific kinesin cellular activities, such as chromosome congregation, axonal transport, etc. Members of the kinesin superfamily are believed to be essential for mitotic and meiotic spindle organization, chromosome segregation, organelle and vesicle transport and many other processes that require microtubule based transport. The common feature of kinesins in the presence of a conserved ca350 amino acid motor domain which harbors the microtubule binding, ATP-hydrolyzing, and force transducing activities (see, e.g., Barton et al. (1996) Proc. Natl. Acad. Sci. USA, 93(5): 1735-1742, and Goldstein, (1993) Annu. Rev. Genet., 27: 319-351). For a review of kinesins (kinesin motors) and kinesin related proteins, see, e.g., Kreis and Vale (1993) Guidebook to the Cytoskeletal and Motor Proteins, Oxford University Press, Oxford and references therein). Kinesin heavy and light chains have been cloned and sequenced from a number of species including human (GenBank X65873).

The terms “kinesin motor inhibitor” or “inhibition of kinesin motor activity” refers to the decrease or elimination of kinesin/microtubule mediated transduction of chemical energy (e.g. as stored in ATP) into mechanical energy (e.g., force generation or movement). Such a decrease can be measured directly, e.g., as in a motility assay, or alternatively can be ascertained by the use of surrogate markers such as a decrease in the ATPase activity of the kinesin protein, and/or a decrease in the affinity and/or specificity of kinesin motor protein-microtubule binding interactions, and/or in a decrease in mitotic activity of a cell or cells. Conversely, a “kinesin motor agonist” or “upregulator of kinesin motor activity” refers to the increase of kinesin/microtubule mediated transduction of chemical energy (e.g. as stored in ATP) into mechanical energy (e.g. force generation or movement).

The term “test compound” refers to a compound whose anti-kinesin motor activity it is desired to determine. Such test compounds may include virtually any molecule or mixture of molecules, alone or in a suitable carrier.

“Modulators of HsCENP-E” refers to modulatory molecules identified using in vitro assays for HsCENP-E activity (e.g., inhibitors and activators or enhancers). Such assays include ATPase activity, microtubule gliding, spindle assembly, microtubule depolymerizing activity, and metaphase arrest. Assays that are treated with a at least one candidate agent at a test concentration are compared to control samples having the candidate agent at a control concentration (which can be zero), to examine the extent of modulation. Control samples are assigned a relative HsCENP-E activity value of 100. Modulation of HsCENP-E is achieved when the HsCENP-E activity value relative to the control is increased or decreased about at least 10%, 20%, 30%, 40%, 50%, 75%, or preferably, at least 100%.

“Treatment” refers to both therapeutic treatment and prophylactic or preventative measures. Those in need of treatment include those already with the disorder as well as those prone to have the disorder or those in which the disorder is to be prevented.

A “disorder” is any condition that would benefit from treatment with the invention protein of the invention. This includes chronic and acute disorders or diseases including those pathological conditions which predispose the mammal to the disorder in question. Disorders include, but are not limited to, those of the cardiovascular system, the nervous system and those involving pain perception.

As used herein, “functional” with respect to a recombinant or heterologous HsCENP-E means that the protein(s) of the invention exhibits an activity attending native CENP-E as assessed by any in vitro or in vivo assay disclosed herein or known to those of skill in the art. Possession of any such activity may be assessed by any method known to those of skill in the art and provided herein is sufficient to designate a peptide as functional. Such activity may be detected as noted supra.

I. Isolated Nucleic Acid Molecule(s)

The invention is based on the discovery of a novel human centromere-associated motor protein (HsCENP-E), the polynucleotides encoding HsCENP-E, and the use of these compositions for the diagnosis, treatment, or prevention of cancer, neurological disorders, and disorders of vesicular transport.

In one aspect, the invention provides an isolated nucleic acid molecule comprising a sequence of nucleotides encoding a kinesin superfamily motor protein, wherein the motor protein has the following properties: (i) the protein's activity includes microtubule stimulated ATPase activity; (ii) the protein has a sequence that has greater than 70% amino acid sequence identity to SEQ ID NO:2 as measured using a sequence comparison algorithm, and (iii) the protein specifically binds to antibodies raised against SEQ ID NO:2.

In a particular embodiment, the invention encompasses a polynucleotide sequence comprising the nucleotide sequence of SEQ ID NO:1, which encodes HsCENP-E thereof.

In another embodiment, the nucleic acid molecule encodes SEQ ID NO:2.

Complementary DNA clones encoding a HsCENP-E of the invention may be prepared from the DNA provided. As well, the polynucleotides of the invention can be obtained from natural sources such as genomic DNA libraries or can be synthesized using well known and commercially available techniques.

“Isolated HsCENP-E nucleic acid molecule” is RNA or DNA containing greater than 16 and preferably 20 or more sequential nucleotide bases that encodes biologically active HsCENP-E or a fragment thereof, is complementary to the RNA or DNA, or hybridizes to the RNA or DNA and remains stably bound under moderate to stringent conditions. This RNA or DNA is free from at least one contaminating source nucleic acid molecule with which it is normally associated in the natural source and preferably substantially free of any other mammalian RNA or DNA. An example of isolated HsCENP-E nucleic acid molecule is RNA or DNA that encodes a biologically active HsCENP-E sharing at least 75%, more preferably at least 80%, still more preferably at least 85%, even more preferably 90%, and most preferably 95% sequence identity with the native CENP-E.

Among particularly preferred embodiments of the invention are polynucleotides encoding HsCENP-E proteins having the amino acid sequence of set out in SEQ ID NO:2 and biologically active fragments thereof.

Preferred embodiments of the invention are polynucleotides that are at least 80% identical over their entire length to a polynucleotide encoding the HsCENP-E protein having the amino acid sequence set out in SEQ ID NO:2, and polynucleotides which are complementary to such polynucleotides. In this regard, polynucleotides at least 80% identical over their entire length to the same are particularly preferred, and those with at least 90% are especially preferred. Alternatively, polynucelotides having at least 97% sequence identity to the sequence set forth in SEQ ID NO:1 are most highly preferred, with at least 99% being the most preferred.

The present invention further relates to polynucleotides that hybridize to the herein above-described sequences. In this regard, the present invention especially relates to polynucleotides which hybridize under stringent conditions to the herein above-described polynucleotides. As herein used, the term “stringent conditions” means hybridization will occur only if there are at least 95% and preferably at least 97% identity between the sequences.

It will be appreciated by those skilled in the art that as a result of the degeneracy of the genetic code, a multitude of polynucleotide sequences encoding HsCENP-E, some bearing minimal similarity to the polynucleotide sequences of any known and naturally occurring gene, may be produced. Thus, the invention also contemplates each and every possible variation of a polynucleotide sequence that could be made by selecting combinations based on possible codon choices. These combinations are made in accordance with the standard triplet genetic code as applied to the polynucleotide sequence of naturally occurring HsCENP-E, and all such variations are to be considered as being specifically disclosed.

As used herein, the term “degenerate” refers to codons that differ in at least one nucleotide from SEQ ID NO:1, but encode the same amino acids as that set forth nucleotide from SEQ ID NO:2 (HsCENP-E).

For example, codons specified by the triplets “UCU”, “UCC”, “UCA”, and “UCG” are degenerate with respect to each other since all four of these codons encode the amino acid serine.

Thus, a nucleotide sequence encoding a HsCENP-E protein may be identical over its entire length to the coding sequence set forth in one of SEQ ID NO:1, or may be a degenerate form of this nucleotide sequence encoding the polypeptide of SEQ ID NO:2, or may be highly identical to a nucleotide sequence that encodes the polypeptide of SEQ ID NO:2.

An exemplary nucleic acid molecule encoding a HsCENP-E protein may be selected from:
(a) DNA encoding the amino acid sequence set forth in SEQ ID NO:2.
(b) DNA that hybridizes to the DNA of (a) under moderately stringent conditions, wherein the DNA encodes biologically active Human HsCENP-E; or
(c) DNA degenerate with respect to either (a) or (b) above, wherein the DNA encodes biologically active Human HsCENP-E.

Another embodiment of the invention contemplates nucleic acid(s) having substantially the same nucleotide sequence as the reference nucleotide sequence that encodes substantially the same amino acid sequence as that set forth in SEQ ID NO:2.

Polynucleotides which are identical or sufficiently identical to a nucleotide sequence contained in SEQ ID NO:1, may be used as hybridization probes for cDNA and genomic DNA or as primers for a nucleic acid amplification (PCR) reaction, to isolate full-length cDNAs and genomic clones encoding polypeptides of the present invention and to isolate cDNA and genomic clones of other genes (including genes encoding homologs and orthologs from species other than human) that have a high sequence similarity to SEQ ID NO:1. Typically these nucleotide sequences are 70% identical, preferably 80% identical, more preferably 90% identical, most preferably 95% identical to that of the referent. The probes or primers will generally comprise at least 15 nucleotides, preferably, at least 30 nucleotides and may have at least 50 nucleotides. Particularly preferred probes will have between 30 and 50 nucleotides.

The skilled artisan will appreciate that, in many cases, an isolated cDNA sequence will be incomplete, in that the region coding for the human protein of the invention is cut short at the 5′ end of the cDNA. This is a consequence of reverse transcriptase, an enzyme with inherently low ‘processivity’ (a measure of the ability of the enzyme to remain attached to the template during the polymerization reaction), failing to complete a DNA copy of the mRNA template during 1st strand cDNA synthesis.

Although nucleotide sequences which encode HsCENP-E and its fragments are preferably capable of hybridizing to the nucleotide sequence of the naturally occurring HsCENP-E under appropriately selected conditions of stringency, it may be advantageous to produce nucleotide sequences encoding HsCENP-E or its derivatives possessing a substantially different codon usage, e.g., inclusion of non-naturally occurring codons. Codons may be selected to increase the rate at which expression of the peptide occurs in a particular prokaryotic or eukaryotic host in accordance with the frequency with which particular codons are utilized by the host. Other reasons for substantially altering the nucleotide sequence encoding HsCENP-E and its derivatives without altering the encoded amino acid sequences include the production of RNA transcripts having more desirable properties, such as a greater half-life, than transcripts produced from the naturally occurring sequence.

The nucleic acid sequences encoding HsCENP-E may be extended utilizing a partial nucleotide sequence and employing various PCR-based methods known in the art to detect upstream sequences, such as promoters and regulatory elements. Various protocols exist to achieve the above object. See e.g., Sarkar, G. (1993) PCR Methods Applic. 2: 318-322; Trnglia, T. et al. (1988) Nucleic Acids Res. 16: 8186); Lagerstrom, M. et al. (1991) PCR Methods Applic. 1: 111-119 Parker, J. D. et al. (1991) Nucleic Acids Res. 19: 3055-306. Other methods for obtaining full-length cDNAs, or extend short cDNAs are well known. See, e.g., Frohman et al., PNAS USA 85, 8998-9002, 1988).

Methods for DNA sequencing are well known in the art and may be used to practice any of the embodiments of the invention. The methods may employ such enzymes as the Klenow fragment of DNA polymerase I, SEQUENASE (US Biochemical, Cleveland Ohio), Taq polymerase (Perkin-Elmer), thermostable T7 polymerase (Amersham Pharmacia Biotech, Piscataway N.J.), or combinations of polymerases and proofreading exonucleases such as those found in the ELONGASE amplification system (Life Technologies, Gaithersburg Md.). Sequencing may be carried out using either ABI 373 or 377 DNA sequencing systems (Perkin-Elmer) or the MEGABACE 1000 DNA sequencing system (Molecular Dynamics, Sunnyvale Calif.). The resulting sequences are analyzed using a variety of algorithms which are well known in the art. (See, e.g., Ausubel, F. M. (1997) Short Protocols in Molecular Biology, John Wiley & Sons, New York N.Y., unit 7.7; Meyers, R. A. (1995) Molecular Biology and Biotechnology, Wiley VCH, New York N.Y., pp. 856-853.)

A polynucleotide encoding a polypeptide of the present invention, including homologs and orthologs from species other than human, may be obtained by a process which comprises the steps of screening an appropriate library under stringent hybridization conditions with a labeled probe having the sequence of SEQ ID NO:1 or a fragment derived therefrom; and isolating full-length cDNA and genomic clones containing the polynucleotide sequence. Such hybridization techniques are well known to the skilled artisan. After screening the mammalian library, positive clones are identified by detecting a hybridization signal; the identified clones are characterized by restriction enzyme mapping and/or DNA sequence analysis, and then examined, by comparison with the sequences set forth herein, to ascertain whether they include DNA encoding the entire invention protein. If the selected clones are incomplete, they may be used to rescreen the same or a different library to obtain overlapping clones. If desired, the library can be rescreened with positive clones until overlapping clones that encode an entire invention protein are obtained. If the library is a cDNA library, then the overlapping clones will include an open reading frame. If the library is genomic, then the overlapping clones may include exons and introns. In both instances, complete clones may be identified by comparison with the DNA and encoded proteins provided herein.

Nucleic acid molecules thus identified can, in turn, be used for producing the HsCENP-E proteins encoded by the nucleic acid molecules, when such nucleic acids are incorporated into a variety of protein expression systems known to those of skill in the art. In addition, such nucleic acid molecules or fragments thereof can be labeled with a readily detectable substituent and used as hybridization probes for assaying for the presence and/or amount of a Human HsCENP-E encoding gene or mRNA transcript in a given sample. The nucleic acid molecules described herein, and fragments thereof, are also useful as primers and/or templates in a PCR reaction for amplifying genes encoding the invention protein described herein.

When screening for full-length cDNAs, it is preferable to use libraries that have been size-selected to include larger cDNAs. Preferred stringent hybridization conditions include overnight incubation at 42° C. In a solution comprising: 50% formamide, 5×SSC (150 mM NaCl, 15 mM trisodium citrate), 50 mM sodium phosphate (pH7.6), 5× Denhardt's solution, 10% dextran sulfate, and 20 microgram/ml denatured, sheared salmon sperm DNA; followed by washing the filters in 0.1×SSC at about 65° C.

An aspect of the invention also includes polynucleotides obtainable by screening an appropriate library under stringent hybridization conditions with a labeled probe having the sequence of SEQ ID NO:1 or a fragment thereof.

The invention nucleic acids can be produced by a variety of methods well-known in the art, e.g., the methods described herein, employing PCR amplification using oligonucleotide primers from various regions of SEQ ID NO:1 and the like. Alternatively, sequences encoding HsCENP-E may be synthesized, in whole or in part, using chemical methods well known in the art. (See, e.g., Caruthers, M. H. et al. (1980) Nucl. Acids Res. Symp. Ser. 215-223, and Hom, T. et al. (1980) Nucl. Acids Res. Symp. Ser. 225-232). Alternatively, HsCENP-E itself or a fragment thereof may be synthesized using chemical methods. For example, peptide synthesis can be performed using various solid-phase techniques. (See, e.g., Roberge, J. Y. et al. (1995) Science 269: 202-204). Automated synthesis may be achieved using the ABI 431A peptide synthesizer (Perkin-Elmer).

In another embodiment of the invention, polynucleotide sequences or fragments thereof which encode a HsCENP-E protein may be cloned in recombinant DNA molecules that direct expression of HsCENP-E, or fragments or functional equivalents thereof, in appropriate host cells. Due to the inherent degeneracy of the genetic code, other DNA sequences which encode substantially the same or a functionally equivalent amino acid sequence may be produced and used to express HsCENP-E.

The nucleotide sequences of the present invention can be engineered using methods generally known in the art in order to alter HsCENP-E-encoding sequences for a variety of purposes including, but not limited to, modification of the cloning, processing, and/or expression of the gene product. DNA shuffling by random fragmentation and PCR reassembly of gene fragments and synthetic polynucleotides may be used to engineer the nucleotide sequences. For example, oligonucleotide-mediated site-directed mutagenesis may be used to introduce mutations that create new restriction sites, alter glycosylation patterns, change codon preference, produce splice variants, and so forth.

Numerous methods are known and available to one skilled in the art to identify cells transformed with nucleic acids of the invention. For example, immunological methods for detecting and measuring the expression of HsCENP-E using either specific polyclonal or monoclonal antibodies are known in the art. Examples of such techniques include enzyme-linked immunosorbent assays (ELISAs), radioimmunoassays (RIAs), and fluorescence activated cell sorting (FACS). A two-site, monoclonal-based immunoassay utilizing monoclonal antibodies reactive to two non-interfering epitopes on HsCENP-E is preferred, but a competitive binding assay may be employed. These and other assays are well known in the art. (See, e.g., Hampton, R. et al. (1990) Serological Methods, a Laboratory Manual, APS Press, St Paul Minn., Sect. IV; Coligan, J. E. et al. (1997) Current Protocols in Immunology, Greene Pub. Associates and Wiley-Interscience, New York N.Y.; and Pound, J. D. (1998) Immunochemical Protocols, Humana Press, Totowa N.J.).

Also provided are antisense polynucleotides having a nucleotide sequence capable of binding specifically with any portion of an mRNA that encodes the invention protein so as to prevent translation of the mRNA. The antisense oligonucleotide may have a sequence capable of binding specifically with any portion of the sequence of the cDNA encoding the invention polypeptides.

A wide variety of labels and conjugation techniques are known by those skilled in the art and may be used in various nucleic acid and amino acid assays. Means for producing labeled hybridization or PCR probes for detecting sequences related to polynucleotides encoding HsCENP-E include oligolabeling, nick translation, end-labeling, or PCR amplification using a labeled nucleotide.

Fragments of a HsCENP-E protein may be produced not only by recombinant production, but also by direct peptide synthesis using solid-phase techniques. (See, e.g., Creighton, supra pp. 55-60). Protein synthesis may be performed by manual techniques or by automation. Automated synthesis may be achieved, for example, using the ABI 431A peptide synthesizer (Perkin-Elmer). Various fragments of HsCENP-E may be synthesized separately and then combined to produce the full length molecule.

As used herein, “expression” refers to the process by which polynucleic acids are transcribed into mRNA and translated into peptides, polypeptides, or proteins. If the polynucleic acid is derived from genomic DNA, expression may, if an appropriate eukaryotic host cell or organism is selected, include splicing of the mRNA.

II. Vectors, Host Cells, Expression etc.

The present invention also relates to vectors, preferably expression vectors, containing a nucleic acid molecule of the invention, e.g., (a) encoding the amino acid sequence of SEQ ID NO:2 or a biologically active fragment thereof, or (b) having a nucleotide sequence comprising the sequence set forth in SEQ ID NO:1 or a portion thereof.

It will be appreciated by those skilled in the art that the design of the expression vector can depend on such factors as the choice of the host cell to be transformed, the level of expression of protein desired, and the like. The expression vectors of the invention can be introduced into host cells to thereby produce proteins or peptides, including fusion proteins or peptides, encoded by nucleic acids as described herein (e.g., HsCENP-E proteins, mutant forms of HsCENP-E proteins, fusion proteins, and the like).

Incorporation of cloned DNA into a suitable expression vector, transfection of eukaryotic cells with a plasmid vector or a combination of plasmid vectors, each encoding one or more distinct genes or with linear DNA, and selection of transfected cells are well known in the art (see, e.g., Sambrook et al. (1989) Molecular Cloning: A Laboratory Manual, Second Edition, Cold Spring Harbor Laboratory Press). Suitable means for introducing (transducing) expression vectors containing invention nucleic acid constructs into host cells to produce transduced recombinant cells (i.e., cells containing recombinant heterologous nucleic acid) are well-known in the art. For a detailed review, see, e.g., Friedmann, 1989, Science, 244: 1275-1281; Mulligan, 1993, Science, 260: 926-932, each of which is incorporated herein by reference in their entirety.

As used herein, the terms “transformation” and “transfection” are intended to refer to a variety of art-recognized techniques for introducing foreign nucleic acid (e.g., DNA) into a host cell. Suitable methods for transforming or transfecting host cells can be found in Sambrook, et al. (Molecular Cloning: A Laboratory Manual. 2nd, ed., Cold Spring Harbor Laboratory, Cold Spring Harbor Laboratory Press, Cold Spring Harbor, N.Y., 1989), and other laboratory manuals.

The recombinant expression vector(s) of the invention comprise a nucleic acid of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990). Regulatory sequences include those which direct constitutive expression of a nucleotide sequence in many types of host cell and those which direct expression of the nucleotide sequence only in certain host cells (e.g., tissue-specific regulatory sequences).

Preferably, a recombinant expression vector of the invention comprises a nucleic acid molecule encoding a kinesin superfamily motor protein, wherein the motor protein has the following properties: (i) the protein's activity includes microtubule stimulated ATPase activity; and (ii) the protein has a sequence that has greater than 70% amino acid sequence identity to SEQ ID NO:2 as measured using a sequence comparison algorithm.

In order to express a biologically active HsCENP-E, the nucleotide sequences encoding HsCENP-E or derivatives thereof may be inserted into an appropriate expression vector, i.e., a vector which contains the necessary elements for transcriptional and translational control of the inserted coding sequence in a suitable host. These elements include regulatory sequences, such as enhancers, constitutive and inducible promoters, and 5′ and 3′ untranslated regions in the vector and in polynucleotide sequences encoding HsCENP-E. Such elements may vary in their strength and specificity. Specific initiation signals may also be used to achieve more efficient translation of sequences encoding HsCENP-E. Such signals include the ATG initiation codon and adjacent sequences, e.g. the Kozak sequence. In cases where sequences encoding HsCENP-E and its initiation codon and upstream regulatory sequences are inserted into the appropriate expression vector, no additional transcriptional or translational control signals may be needed. However, in cases where only coding sequence, or a fragment thereof, is inserted, exogenous translational control signals including an in-frame ATG initiation codon should be provided by the vector. Exogenous translational elements and initiation codons may be of various origins, both natural and synthetic. The efficiency of expression may be enhanced by the inclusion of enhancers appropriate for the particular host cell system used. (See, e.g., Scharf, D. et al. (1994) Results Probl. Cell Differ. 20: 125-162).

Generally, the DNA sequence that will ultimately be expressed is joined to an expression vector by cleaving the DNA sequence and the expression vector with one or more restriction enzymes and then ligating the fragments together.

Procedures for restriction enzyme digestion are well known to those of ordinary skill in the art. Procedures for ligating the heterologous nucleic acid, the promoter, terminator and other elements, respectively, and to insert them into suitable cloning vehicles containing the information necessary for replication, are well known to persons skilled in the art (vide e.g., Sambrook et al., 1989; inter alia).

Exemplary methods of transduction include, e.g., infection employing viral vectors (see, e.g., U.S. Pat. Nos. 4,405,712 and 4,650,764), calcium phosphate transfection (U.S. Pat. Nos. 4,399,216 and 4,634,665), dextran sulfate transfection, electroporation, lipofection (see, e.g., U.S. Pat. Nos. 4,394,448 and 4,619,794), cytofection, particle bead bombardment, and the like. The heterologous nucleic acid can optionally include sequences which allow for its extrachromosomal (i.e., episomal) maintenance, or the heterologous nucleic acid can be donor nucleic acid that integrates into the genome of the host. Recombinant cells can then be cultured under conditions whereby the invention protein(s) encoded by the DNA is (are) expressed. Preferred cells include mammalian cells (e.g., HEK 293, CHO and Ltk⁻ cells), yeast cells (e.g., methylotrophic yeast cells, such as Pichia pastoris), bacterial cells (e.g., Escherichia coli), and the like.

As used herein, “heterologous or foreign DNA and/or RNA” are used interchangeably and refer to DNA or RNA that does not occur naturally as part of the genome of the cell in which it is present or to DNA or RNA which is found in a location or locations in the genome that differ from that in which it occurs in nature. Typically, heterologous or foreign DNA and RNA refers to DNA or RNA that is not endogenous to the host cell and has been artificially introduced into the cell. Examples of heterologous DNA include DNA that encodes the invention proteins.

Methods which are well known to those skilled in the art may be used to construct expression vectors containing sequences encoding HsCENP-E and appropriate transcriptional and translational control elements. These methods include in vitro recombinant DNA techniques, synthetic techniques, and in vivo genetic recombination. (See, e.g., Sambrook, J. et al. (1989) Molecular Cloning, A Laboratory Manual, Cold Spring Harbor Press, Plainview N.Y., ch. 4, 8, and 16-17; Ausubel, F. M. et al. (1995) Current Protocols in Molecular Biology, John Wiley & Sons, New York N.Y., ch. 9, 13, and 16).

Another aspect of the invention pertains to host cells into which a recombinant expression vector of the invention has been introduced. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A variety of expression vector/host systems may be utilized to contain and express sequences encoding HsCENP-E. These include, but are not limited to, microorganisms such as bacteria transformed with recombinant bacteriophage, plasmid, or cosmid DNA expression vectors; yeast transformed with yeast expression vectors; insect cell systems infected with viral expression vectors (e.g., baculovirus); amphibian cells (e.g., Xenopus laevis oocytes); plant cell systems transformed with viral expression vectors (e.g., cauliflower mosaic virus, CaMV, or tobacco mosaic virus, TMV) or with bacterial expression vectors (e.g., Ti or pBR322 plasmids); or mammalian cells such as Chinese hamster ovary cells (CHO) or COS cells Or animal cell systems. The invention is not limited by the host cell employed. Exemplary cells for expressing injected RNA transcripts include Xenopus laevis oocytes. Generally, any system or vector which is able to maintain, propagate or express a polynucleotide to produce a polypeptide in a host may be used. The appropriate nucleotide sequence may be inserted into an expression system by any of a variety of well-known and routine techniques, such as, for example, those set forth in Sambrook et al., MOLECULAR CLONING, A LABORATORY MANUAL (supra).

In bacterial systems, a number of cloning and expression vectors may be selected depending upon the use intended for polynucleotide sequences encoding HsCENP-E. For example, routine cloning, subcloning, and propagation of polynucleotide sequences encoding HsCENP-E can be achieved using a multifunctional E. coli vector such as pBLUESCRIPT (Stratagene, La Jolla Calif.) or pSPORT1 plasmid (Life Technologies). Ligation of sequences encoding HsCENP-E into the vector's multiple cloning site disrupts the lacZ gene, allowing a colorimetric screening procedure for identification of transformed bacteria containing recombinant molecules. In addition, these vectors may be useful for in vitro transcription, dideoxy sequencing, single strand rescue with helper phage, and creation of nested deletions in the cloned sequence. (See, e.g., Van Heeke, G. and S. M. Schuster (1989) J. Biol. Chem. 264: 5503-5509). When large quantities of HsCENP-E are needed, e.g. for the production of antibodies, vectors which direct high level expression of HsCENP-E may be used. For example, vectors containing the strong, inducible T5 or T7 bacteriophage promoter may be used.

Exemplary expression vectors for transformation of E. coli prokaryotic cells include the pET expression vectors (Novagen, Madison, Wis., see U.S. Pat. No. 4,952,496), e.g., pETlla, which contains the T7 promoter, T7 terminator, the inducible E. coli lac operator, and the lac repressor gene; and pET 12a-c, which contains the T7 promoter, T7 terminator, and the E. coli ompT secretion signal. Another such vector is the pIN-IIIompA2 (see Duffaud et al., Meth. in Enzymology, 153: 492-507, 1987), which contains the lpp promoter, the lacUV5 promoter operator, the ompA secretion signal, and the lac repressor gene.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith, D. B. and Johnson, K. S. (1988) Gene 67: 31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

One strategy to maximize recombinant protein expression in E. coli is to express the protein in a host bacteria with an impaired capacity to proteolytically cleave the recombinant protein (Gottesman, S., Gene Expression Technology: Methods in Enzymology 185, Academic Press, San Diego, Calif. (1990) 119-128). Another strategy is to alter the nucleic acid sequence of the nucleic acid to be inserted into an expression vector so that the individual codons for each amino acid are those preferentially utilized in E. coli (Wada et al., (1992) Nucleic Acids Res. 20: 2111-2118). Such alteration of nucleic acid sequences of the invention can be carried out by standard DNA synthesis techniques.

Fungal cells, including species of yeast or filamentous fungi (e.g., Aspergillus spp., Neurospora spp.) may be used as host cells within the present invention.

Exemplary yeast cells include Saccharomyces cerevisiae, or common baker's yeast, which is the most commonly used among eukaryotic microorganisms. Other strains are available and may be substituted for the baker's yeast. The term “yeast”, as used herein, includes not only yeast in a strictly taxonomic sense, i.e., unicellular organisms, but also yeast-like multicellular fungi or filamentous fungi.

Representative vectors that are operable in yeast cells are well known. For expression in Saccharomyces, the plasmid YRp7, for example, (Stinchcomb, et al, Nature, 282: 39 (1979); Kingsman et al, Gene, 7: 141 (1979); Tschemper, et al, Gene, 10: 157 (1980)) is preferred. Other representative promoters that are functional in yeast and required for the expression of a heterologous nucleic acid in yeast include the promoters for metallothionein, 3-phosphoglycerate kinase (Hitzeman et al., J. Biol. Chem. 255, 2073 (1980) or other glycolytic enzymes (Hess et al., J. Adv. Enzyme Req. 7, 149 (1968); and Holland et al. Biochemistry 17, 4900 (1978)), such as enolase, glyceraldehyde-3-phosphate dehydrogenase, hexokinase, pyruvate decarboxylase, phospho-fructokinase, glucose-6-phosphate isomerase, 3-phosphoglycerate mutase, pyruvate kinase, triosephosphate isomerase, phospho-glucose isomerase, and glucokinase.

Examples of vectors for expression in yeast e.g., S. cerevisiae include pYepSec1 (Baldari, et al., EMBO J. 6: 229-234 (1987)), pMFa (Kuijan et al., Cell 30: 933-943(1982)), pJRY88 (Schultz et al., Gene 54: 113-123 (1987)), and pYES2 (Invitrogen Corporation, San Diego, Calif.). A number of other vectors are known that are capable of expressing recombinant proteins in yeast. Representative examples include YEP24, YIP5, YEP51, YEP52, and YRP17, all of which are suitable cloning and expression vehicles useful in the introduction of genetic constructs into S. cerevisiae. See Broach et al. (1983) in Experimental Manipulation of Gene Expression, ed. M. Inouye Academic Press, p. 83, incorporated by reference herein).

Additional vectors, promoters and terminators for use in expressing the HsCENP-E protein(s) of the invention in yeast are well known in the art and are reviewed by, for example, Emr, Meth. Enzymol. 185: 231-279, (1990), incorporated herein by reference. The HsCENP-E proteins of the invention may be expressed in Aspergillus spp. (McKnight and Upshall, described in U.S. Pat. No. 4,935,349, which is incorporated herein by reference). Useful promoters include those derived from Aspergillus nidulans glycolytic genes, such as the ADH3 promoter (McKnight et al., EMBO J. 4: 2093-2099, 1985) and the tpiA promoter. An example of a suitable terminator is the ADH3 terminator (McKnight et al., ibid.). Techniques for transforming fungi are well known in the literature, and have been described, for instance by Beggs (ibid.), Hinnen et al. (Proc. Natl. Acad. Sci. USA 75: 1929-1933, 1978), Yelton et al. (Proc. Natl. Acad. Sci. USA 81: 1740-1747, 1984), and Russell (Nature 301: 167-169, 1983) each of which are incorporated herein by reference.

Alternatively, the HsCENP-E proteins can be expressed in insect cells using baculovirus expression vectors. Baculovirus vectors available for expression of proteins in cultured insect cells (e.g., Sf9 cells) include the pAc series (Smith et al. (1983) Mol. Cell Biol. 3: 2156-2165) and the pVL series (Lucklow and Summers (1989) Virology 170: 31-39).

Eukaryotic cells in which DNA or RNA may be introduced include any cells that are transfectable by such DNA or RNA or into which such DNA or RNA may be injected. Preferred cells are those that can be transiently or stably transfected and also express the DNA and RNA. Presently most preferred cells are those that can express recombinant or heterologous HsCENP-E encoded by the nucleic acid molecule(s) of the invention. Such cells may be identified empirically or selected from among those known to be readily transfected or injected.

Host cells transformed with nucleotide sequences encoding HsCENP-E may be cultured under conditions suitable for the expression and recovery of the protein from cell culture. The protein produced by a transformed cell may be secreted or retained intracellularly depending on the sequence and/or the vector used. As will be understood by those of skill in the art, expression vectors containing polynucleotides which encode HsCENP-E may be designed to contain signal sequences which direct secretion.

The proteins of the present invention can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Most preferably, high performance liquid chromatography is employed for purification. Well known techniques for refolding proteins may be employed to regenerate active conformation when the polypeptide is denatured during isolation and/or purification. Methods of protein purification are known in the art (see generally, Scopes, R., Protein Purification, Pringer-Verlag, N.Y. (1982), which is incorporated herein by reference) and may be applied to the purification of the HsCENP-E protein and particularly the recombinantly produced HsCENP-E protein described herein.

Nucleic acid molecules of the invention may be stably incorporated into cells or may be transiently introduced using methods known in the art. Stably transfected mammalian cells may be prepared by transfecting cells with an expression vector comprising a sequence of nucleotides that encodes the invention proteins, i.e., either Human HsCENP-E, in conjunction with a selectable marker gene (such as, for example, the gene for thymidine kinase, dihydrofolate reductase, neomycin resistance, and the like), and growing the transfected cells under conditions selective for cells expressing the marker gene. Cells stably transfected with the introduced nucleic acid can be identified by drug selection (e.g., cells that have incorporated the selectable marker gene will survive, while the other cells die).

To prepare transient transfectants, mammalian cells are transfected with a reporter gene (such as the E. coli .beta.-galactosidase gene) to monitor transfection efficiency. The precise amounts and ratios of DNA encoding the invention proteins may be empirically determined and optimized for a particular cell and assay conditions. Selectable marker genes are typically not included in the transient transfections because the transfectants are typically not grown under selective conditions, and are usually analyzed within a few days after transfection.

“Cell,” “cell line,” and “cell culture” are used interchangeably herein and such designations include all progeny of a cell or cell line. Thus, for example, terms like “transformants” and “transformed cells” include the primary subject cell and cultures derived therefrom without regard for the number of transfers. It is also understood that all progeny may not be precisely identical in DNA content, due to deliberate or inadvertent mutations. Mutant progeny that have the same function or biological activity as screened for in the originally transformed cell are included. Where distinct designations are intended, it will be clear from the context.

In other embodiments, mRNA may be produced by in vitro transcription of DNA encoding the invention protein. This mRNA can then be injected into Xenopus oocytes where the RNA directs the synthesis of the invention protein. Alternatively, the invention-encoding DNA can be directly injected into oocytes for expression of a functional invention protein. The transfected mammalian cells or injected oocytes may then be used in the methods of drug screening provided herein.

As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions. It is implied, although not always explicitly stated, that these expression vectors must be replicable in the host organisms either as episomes or as an integral part of the chromosomal DNA. Clearly a lack of replicability would render them effectively inoperable.

In sum, “expression vector” is given a functional definition, and any DNA sequence which is capable of effecting expression of a specified DNA code disposed therein is included in this term as it is applied to the specified sequence. In general, expression vectors of utility in recombinant DNA techniques are often in the form of “plasmids” which refer to circular double stranded DNA loops that, in their vector form are not bound to the chromosome.

As used herein, the term “expression” refers to any number of steps comprising the process by which nucleic acid molecules are transcribed into RNA, and (optionally) translated into peptides, polypeptides, or proteins. If the polynucleic acid is derived from genomic DNA, expression may, if an appropriate eukaryotic host cell or organism is selected, include splicing of the RNA.

Thus, an expression vector refers to a recombinant DNA or RNA construct, such as a plasmid, a phage, recombinant virus or other vector that, upon introduction into an appropriate host cell, results in expression of the inserted DNA. Appropriate expression vectors are well known to those of skill in the art and include those that are replicable in eukaryotic cells and/or prokaryotic cells and those that remain episomal or those which integrate into the host cell genome.

A vector can be maintained in the host cell as an extrachromosomal element where it replicates and produces additional copies of the nucleic acid molecules. Alternatively, the vector may integrate into the host cell genome and produce additional copies of the nucleic acid molecules when the host cell replicates.

Improvements in DNA vectors have also been made and are likely applicable to all of the non-viral delivery systems. These include the use of supercoiled minicircles reported by RPR Gencell (which do not have bacterial origins of replication nor antibiotic resistance genes and thus are potentially safer as they exhibit a high level of biological containment), episomal expression vectors as developed by Copernicus Gene Systems Inc (replicating episomal expression systems where the plasmid amplifies within the nucleus but outside the chromosome and thus avoids genome integration events) and T7 systems as developed by Progenitor (a strictly a cytoplasmic expression vector in which the vector itself expresses phage T7 RNA polymerase and the therapeutic gene is driven from a second T7 promoter, using the polymerase generated by the first promoter). Other, more general improvements to DNA vector technology include use of cis-acting elements to effect high levels of expression (Vical), sequences derived from alphoid repeat DNA to supply once-per-cell-cycle replication and nuclear targeting sequences (from EBNA-1 gene (Calos at Stanford, with Megabios); SV40 early promoter/enhancer or peptide sequences attached to the DNA).

It is noteworthy that transcription of a heterologous nucleic acid molecule encoding the target protein or gene product of interest (the amino acid sequence of SEQ ID NO:2, by higher eukaryotes can be increased by inserting an enhancer sequence into the vector. Enhancers are cis-acting elements of DNA, usually about from 10 to 300 bp that act on a promoter to increase its transcription. Examples including the SV40 enhancer on the late side of the replication origin (bp 100 to 270), a cytomegalovirus early promoter enhancer, the polyoma enhancer on the late side of the replication origin, and adenovirus enhancers.

Immunological methods for detecting and measuring the expression of HsCENP-E proteins of the invention using either specific polyclonal or monoclonal antibodies are known in the art. Examples of such techniques include enzyme-linked immunosorbent assays (ELISAs), radioimmunoassays (RIAs), and fluorescence activated cell sorting (FACS). A two-site, monoclonal-based immunoassay utilizing monoclonal antibodies reactive to two non-interfering epitopes on HsCENP-E is preferred, but a competitive binding assay may be employed. These and other assays are well known in the art. (See, e.g., Hampton, R. et al. (1990) Serological Methods, a Laboratory Manual, APS Press, St Paul Minn., Sect. IV; Coligan, J. E. et al. (1997) Current Protocols in Immunology, Greene Pub. Associates and Wiley-Interscience, New York N.Y.; and Pound, J. D. (1998) Immunochemical Protocols, Humana Press, Totowa N.J.).

A wide variety of labels and conjugation techniques are known by those skilled in the art and may be used in various nucleic acid and amino acid assays. Means for producing labeled hybridization or PCR probes for detecting sequences related to polynucleotides encoding CENP-E include oligolabeling, nick translation, end-labeling, or PCR amplification using a labeled nucleotide. Alternatively, the sequences encoding HsCENP-E protein of the invention, or any fragments thereof, may be cloned into a vector for the production of an mRNA probe. Such vectors are known in the art, are commercially available, and may be used to synthesize RNA probes in vitro by addition of an appropriate RNA polymerase such as T7, T3, or SP6 and labeled nucleotides. These procedures may be conducted using a variety of commercially available kits, such as those provided by Amersham Pharmacia Biotech, Promega (Madison Wis.), and US Biochemical. Suitable reporter molecules or labels which may be used for ease of detection include radionuclides, enzymes, fluorescent, chemiluminescent, or chromogenic agents, as well as substrates, cofactors, inhibitors, magnetic particles, and the like.

Transfected host cells can be identified in numerous ways. For example, the presence of a heterologous nucleic acid molecule e.g., a DNA molecule encoding the amino acid of SEQ ID NO:2 or a portion thereof may be determined by introducing a labeled-oligonucleotide probe that is sufficiently complimentary to a portion of the nucleotide sequence of the heterologous DNA such as to form a complex (hybrid) with the portion of the heterologous DNA sequence, at moderately stringent condition. Identification of transfected or transfected cells is achieved via identification of the hybrid/complex.

III. Substantially Pure HsCENP-E Proteins/Polypeptides and Antibodies

An aspect of the invention pertains to substantially pure HsCENP-E proteins, and biologically active fragments thereof, as well as polypeptide fragments suitable for use as immunogens to raise anti-HsCENP-E antibodies.

An “anti-HsCENP-E” antibody is an antibody or antibody fragment that specifically binds a polypeptide encoded by the HsCENP-E gene, cDNA, or a subsequence thereof.

The HsCENP-E protein(s) of the invention is defined herein to be any polypeptide sequence that possesses at least one biological property (as defined below) of a naturally occurring polypeptide, e.g., wild type HsCENP-E. This definition encompasses not only the polypeptide isolated from a native CENP-E source, but also the polypeptide prepared by recombinant or synthetic methods. It also includes variant forms including functional derivatives, alleles, isoforms and analogues thereof.

In one aspect, HsCENP-E can be defined by having at least one or preferably more than one of the following functional and structural characteristics. Functionally, HsCENP-E will have microtubule-stimulated ATPase activity, and microtubule motor activity that is ATP dependent. HsCENP-E activity can also be described in terms of its ability to bind microtubules. As well, HsCENP-E can be defined by its ability to bind to polyclonal antibodies generated against a motor domain, tail domain or other fragment of native CENP-E.

In another aspect, the invention features a substantially pure polypeptide comprising the amino acid sequence of SEQ ID NO:2 or a fragment thereof and more particularly the motor domain of the amino acid sequence of SEQ ID NO:2 or a fragment thereof.

In one embodiment, HsCENP-E proteins can be isolated from cells or tissue sources by an appropriate purification scheme using standard protein purification techniques. In another embodiment, HsCENP-E proteins are produced by recombinant DNA techniques. Alternative to recombinant expression, a HsCENP-E protein or polypeptide can be synthesized chemically using standard peptide synthesis techniques.

An “isolated” or “purified” protein or biologically active fragment thereof is substantially free of cellular material or other contaminating proteins from the cell or tissue source from which the HsCENP-E protein is derived, or substantially free from chemical precursors or other chemicals when chemically synthesized. The language “substantially free of cellular material” includes preparations of HsCENP-E protein in which the protein is separated from cellular components of the cells from which it is isolated or recombinantly produced. Consequently, use of the terms “isolated” and/or “purified” as used herein as a modifier of polypeptides or proteins means that the polypeptides or proteins so designated have been produced in such form by the hand of man, and thus are separated from their native in vivo cellular environment. As a result, the recombinant polypeptides and proteins of the invention are useful in ways described herein that the DNAs, RNAs, polypeptides or proteins as they naturally occur are not.

“Biologically active fragments” of a HsCENP-E protein are a portion of a naturally occurring mature full-length HsCENP-E sequence or the protein of SEQ ID NO:2 having one or more amino acid residues deleted. As such, fragments include peptides comprising amino acid sequences sufficiently identical to or derived from the amino acid sequence of the HsCENP-E protein, e.g., the amino acid sequence shown in SEQ ID NO:2, which include less amino acids than the full length HsCENP-E proteins, and exhibit at least one activity of a HsCENP-E protein. The deleted amino acid residue(s) may occur anywhere in the polypeptide, including at either the N-terminal or C-terminal end or internally. Typically, biologically active fragments will share at least one biological property in common with the human or wild type HsCENP-E., e.g., ATP binding domain etc. HsCENP-E fragments typically will have a consecutive sequence of at least 10, 15, 20, 25, 30, or 40 amino acid residues that are identical to the sequences of the HsCENP-E isolated from a mammal. Thus, a “HsCENP-E fragment” is a portion of a naturally occurring mature full-length HsCENP-E sequence having one or more amino acid residues or carbohydrate units deleted.

In one embodiment, a biologically active fragment of a HsCENP-E protein (invention protein of SEQ ID NO:2) comprises at least one ATP binding domain. In another embodiment, a biologically active fragment of a HsCENP-E protein of the invention comprises at least one motor domain.

Preferred fragments include, for example, truncation polypeptides having the amino acid sequence of the HsCENP-E protein disclosed herein, except for deletion of a continuous series of residues that includes the amino terminus, or a continuous series of residues that includes the carboxyl terminus or deletion of two continuous series of residues, one including the amino terminus and one including the carboxyl terminus. It is to be understood that a preferred biologically active fragment include an ATP binding domain and a motor domain. Other biologically active fragments, in which other regions of the protein are deleted, can be prepared by recombinant techniques and evaluated for one or more of the functional activities of a native CENP-E protein.

In another embodiment, the HsCENP-E protein has an amino acid sequence shown in SEQ ID NO:2.

The invention also encompasses variants of a HsCENP-E protein. As used herein, a “variant” of the HsCENP-E protein refers to a polypeptide having an amino acid sequence with one or more amino acid substitutions, insertions, and/or deletions compared to the sequence of the invention protein. Generally, differences are limited so that the sequences of the reference (invention protein) and the variant are closely similar overall, and in many regions, identical. Such variants are generally biologically active and necessarily have less than 100% sequence identity with the polypeptide of interest.

Another aspect of the invention proposes a HsCENP-E protein having an amino acid sequence sufficiently identical to the amino acid sequence of SEQ ID NO:2, or encoded by a nucleotide sequence sufficiently identical to SEQ ID NO:1.

As used herein, the term “sufficiently identical” refers to a first amino acid or nucleotide sequence which contains a sufficient or minimum number of identical or equivalent (e.g., an amino acid residue which has a similar side chain) amino acid residues or nucleotides to a second amino acid or nucleotide sequence such that the first and second amino acid or nucleotide sequences share common structural domains or motifs and/or a common functional activity. For example, amino acid or nucleotide sequences which share common structural domains or biological functions have at least 70%-80%, and even more preferably 90-95% identity across the amino acid sequences of the domains or over the entire length of the polypeptide or polynucleotide and contain at least one and preferably two structural domains, are defined herein as sufficiently identical.

Amino-acid substitutions are preferably substitutions of single amino-acid residues. Preferred variants are those that vary from the reference by conservative amino acid substitutions—i.e., those that substitute a residue with another of like characteristics. Typical such substitutions are among Ala, Val, Leu and Ile; among Ser and Thr; among the acidic residues Asp and Glu; among Asn and Gln; and among the basic residues Lys and Arg; or aromatic residues Phe and Tyr. Particularly preferred are variants in which several, 5-10, 1-5, or 1-2 amino acids are substituted, deleted, or added in any combination.

In accordance with the above, preferred proteins are HsCENP-E proteins having at least one ATP binding domain and preferably at least one other HsCENP-E activity. Other preferred proteins are HsCENP-E proteins having at least one ATP binding domain, and are, preferably, encoded by a nucleic acid molecule having a nucleotide sequence which hybridizes under stringent hybridization conditions to the nucleic acid molecule comprising the nucleotide sequence of SEQ ID NO:1.

In other embodiments, the HsCENP-E protein is substantially homologous to SEQ ID NO:2, and retains the functional activity of the protein of SEQ ID NO:2, yet differs in amino acid sequence due to natural allelic variation or mutagenesis, as described herein.

Variants of HsCENP-E which has at least 80% identity to the polypeptide of SEQ ID NO:2 or and more preferably at least 85% identity, and still more preferably at least 90% identity, and even still more preferably at least 95% identity to SEQ ID NO:2 are also encompassed by the invention. Preferably, all of these polypeptides retain the biological activity of the protein disclosed herein (SEQ ID NO:2), including antigenic activity.

In other embodiments, the HsCENP-E protein is substantially homologous to SEQ ID NO:2, and retains the functional activity of the protein of SEQ ID NO:2, yet differs in amino acid sequence due to natural allelic variation or mutagenesis, as described herein.

To determine the percent identity of two amino acid sequences or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 70%, or 80%, or 90% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch (J. Mol. Biol. (48): 444-453 (1970)) algorithm which has been incorporated into the GAP program in the GCG software package (available at http://www.gcg.com), using either a Blosum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6.

In another embodiment, the percent identity between two amino acid or nucleotide sequences is determined using the algorithm of E. Meyers and W. Miller (CABIOS, 4: 11-17 (1989)) which has been incorporated into the ALIGN program (version 2.0), using a PAM120 weight residue table, a gap length penalty of 12 and a gap penalty of 4.

The nucleic acid and protein sequences of the present invention can further be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215: 403-10. BLAST nucleotide searches can be performed with the NBLAST program, score=100, wordlength=12 to obtain nucleotide sequences homologous to HsCENP-E nucleic acid molecules of the invention. BLAST protein searches can be performed with the XBLAST program, score=50, wordlength=3 to obtain amino acid sequences homologous to HsCENP-E protein molecules of the invention. To obtain gapped alignments for comparison purposes, Gapped BLAST can be utilized as described in Altschul et al., (1997) Nucleic Acids Res. 25(17): 3389-3402. When utilizing BLAST and Gapped BLAST programs, the default parameters of the respective programs (e.g., XBLAST and NBLAST) can be used. See http://www.ncbi.nlm.nih.gov.

The invention also provides HsCENP-E chimeric or fusion proteins. As used herein, a HsCENP-E “chimeric protein” or “fusion protein” comprises a HsCENP-E protein operatively linked to a non-HsCENP-E protein. An “HsCENP-E protein” is as defined supra, whereas a “non-HsCENP-E protein” refers to a polypeptide having an amino acid sequence corresponding to a protein which is not substantially homologous to the HsCENP-E protein, e.g., a protein which is different from the HsCENP-E protein and which is derived from the same or a different organism. Within a HsCENP-E fusion protein the HsCENP-E protein can correspond to all or a portion of a HsCENP-E protein.

In a preferred embodiment, a HsCENP-E fusion protein comprises at least one biologically active portion of a HsCENP-E protein. In another preferred embodiment, a HsCENP-E fusion protein comprises at least two biologically active portions of a HsCENP-E protein.

Within the fusion protein, the term “operatively linked” is intended to indicate that the HsCENP-E protein and the non-HsCENP-E protein are fused in frame to each other. The non-HsCENP-E protein can be fused to the N-terminus or C-terminus of the HsCENP-E protein.

In another embodiment, the fusion protein is a HsCENP-E protein containing a heterologous signal sequence at its N-terminus. In certain host cells (e.g., mammalian host cells), expression and/or secretion of HsCENP-E can be increased through use of a heterologous signal sequence.

Use of HsCENP-E fusion proteins may be useful therapeutically for the treatment of HsCENP-E related/associated disorders such as Cancer.

Moreover, the HsCENP-E-fusion proteins of the invention can be used as immunogens to produce anti-HsCENP-E antibodies in a subject, to purify HsCENP-E ligands and in screening assays to identify molecules which inhibit the interaction of HsCENP-E with a HsCENP-E binding partner or substrate.

Preferably, a HsCENP-E chimeric or fusion protein of the invention is produced by standard recombinant DNA techniques. One such technique is described in “Current Protocols in Molecular Biology”, eds. Ausubel et al. John Wiley & Sons: 1992).

The present invention also pertains to variants of the HsCENP-E proteins, which function as either HsCENP-E agonists (mimetics) or as HsCENP-E antagonists. Variants of the HsCENP-E proteins can be generated by mutagenesis, e.g., discrete point mutation or truncation of a HsCENP-E protein.

An agonist of the HsCENP-E proteins can retain substantially the same, or a subset, of the biological activities of the naturally occurring form of a HsCENP-E protein. An antagonist of a HsCENP-E protein can inhibit one or more of the activities of the naturally occurring form of the HsCENP-E protein.

In furtherance of the above, an embodiment of the invention contemplates treatment of a subject with a variant HsCENP-E protein having a subset of the biological activities of the naturally occurring form of the protein and having fewer side effects in a subject relative to treatment with the naturally occurring form of the HsCENP-E protein.

A variegated library of HsCENP-E variants is generated by combinatorial mutagenesis at the nucleic acid level and is encoded by a variegated gene library. A variegated library of HsCENP-E variants can be produced by, for example, enzymatically ligating a mixture of synthetic polynucleotides into gene sequences such that a degenerate set of potential HsCENP-E sequences is expressible as individual polypeptides, or alternatively, as a set of larger fusion proteins (e.g., for phage display) containing the set of HsCENP-E sequences therein. There are a variety of methods which can be used to produce libraries of potential HsCENP-E variants from a degenerate oligonucleotide sequence. Methods for synthesizing degenerate polynucleotides are known in the art (see, e.g., Narang, S. A. (1983) Tetrahedron 39: 3; Itakura et al. (1984) Annu. Rev. Biochem. 53: 323; Itakura et al. (1984) Science 198: 1056; Ike et al. (1983) Nucleic Acid Res. 11: 477.

In addition, libraries of fragments of a HsCENP-E protein coding sequence can be used to generate a variegated population of HsCENP-E fragments for screening and subsequent selection of variants of a HsCENP-E protein.

For example, a library of coding sequence fragments can be generated by treating a double stranded PCR fragment of a HsCENP-E coding sequence with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes N-terminal, C-terminal and internal fragments of various sizes of the HsCENP-E protein.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of HsCENP-E proteins. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recrusive ensemble mutagenesis (REM), a new technique which enhances the frequency of functional mutants in the libraries, can be used in combination with the screening assays to identify HsCENP-E variants (Arkin and Yourvan (1992) Proc. Natl. Acad. Sci. USA 89: 7811-7815; Delgrave et al. (1993) Protein Engineering 6(3): 327-331). See also U.S. Patent Publication No. 2002/0006664 A1 (“reverse transfection”) Alternatively, a plurality of nucleic acid molecules, each or a collection thereof encoding various HsCENP-E mutants can be used to transfect a plurality of cells according to the MAGEC method disclosed in Provisional Application, number unknown, filed Apr. 16, 2002, assigned to Merck & Co., Inc.

In a representative embodiment, cell based assays can be exploited to analyze a variegated HsCENP-E library. For example, a library of expression vectors can be transfected into a cell line which ordinarily possesses a HsCENP-E mediated activity. The effect of the HsCENP-E mutant on the HsCENP-E mediated activity, e.g., cell proliferation can then be detected by any of a number of conventional assays. Plasmid DNA can then be recovered from the cells which score for inhibition and the individual clones further characterized.

In another aspect, the substantially pure HsCENP-E protein of the invention (SEQ ID NO:2), or a fragment thereof, can be used as an immunogen to generate antibodies that bind HsCENP-E using standard techniques for polyclonal and monoclonal antibody preparation. A full-length HsCENP-E protein can be used or, alternatively, the invention provides antigenic peptide fragments of HsCENP-E for use as immunogens.

The antigenic peptide fragment of the herein disclosed HsCENP-E protein comprises at least 8 amino acid residues of the amino acid sequence shown in SEQ ID NO:2 and encompasses an epitope of HsCENP-E such that an antibody raised against the peptide forms a specific immune complex with HsCENP-E. Preferably, the antigenic peptide comprises at least 10 amino acid residues, more preferably at least 15 amino acid residues, even more preferably at least 20 amino acid residues, and most preferably at least 30 amino acid residues.

Preferred epitopes encompassed by the antigenic peptide are regions of HsCENP-E that are located on the surface of the protein, e.g., hydrophilic regions, as well as regions with high antigenicity.

Consequently, an aspect of the invention pertains to anti-HsCENP-E antibodies. The term “antibody” is used in the broadest sense and specifically covers single monoclonal antibodies (including agonist and antagonist antibodies) and antibody compositions with polyepitopic specificity. In general, the term refers to immunoglobulin molecules and antigenically or immunologically active fragments of immunoglobulin molecules, i.e., molecules that contain an antigen binding site which specifically binds (immunoreacts with) an antigen, such as HsCENP-E. Representative examples of immunologically active fragments of immunoglobulin molecules include F(ab) and F(ab).₂ fragments which can be generated by treating the antibody with an enzyme such as pepsin.

Methods of making antibodies are well known. Various hosts including goats, rabbits, rats, mice, humans, and others may be immunized by injection with HsCENP-E or “Immunologically active fragment(s)” thereof for producing the antibodies. “Immunologically active fragment(s)” of HsCENP-E refer to those fragments which are capable of eliciting an immune response can also be used as immunogens to produce antibodies immunospecific for the HsCENP-E protein(s) of the invention. Such fragments are those proteins that are capable of raising HsCENP-E-specific antibodies in a target immune system (e.g., murine or rabbit) or of competing with native CENP-E for binding to HsCENP-E-specific antibodies, and is thus useful in immunoassays for the presence of HsCENP-E peptides in a biological sample. Such immunologically active fragments typically have a minimum size of 8 to 11 consecutive amino acids. As well, it is preferable that the immunologically active fragment(s) be identical to a portion of the amino acid sequence of the native human CENP-E protein and contain the entire amino acid sequence of a small, naturally occurring molecule. Alternatively, short stretches of HsCENP-E amino acids may be fused with those of another protein to form chimeric entities, and antibodies to the chimeric entity may then produced.

Depending on the host species, various adjuvants may be used to increase immunological response. Such adjuvants include, but are not limited to, Freund's, mineral gels such as aluminum hydroxide, and surface active substances such as lysolecithin, pluronic polyols, polyanions, peptides, oil emulsions, KILH, and dinitrophenol. Among adjuvants used in humans, BCG (bacilli Calmette-Guerin) and Corynabacterium parvum are especially preferable.

An appropriate immunogenic preparation can contain, for example, recombinantly expressed HsCENP-E protein or a chemically synthesized HsCENP-E protein. The preparation can further include an adjuvant, such as Freund's complete or incomplete adjuvant, or similar immunostimulatory agent. Immunization of a suitable subject with an immunogenic HsCENP-E preparation induces a polyclonal anti-HsCENP-E antibody response.

Polyclonal anti-HsCENP-E antibodies can be prepared as described above by immunizing a suitable subject with a HsCENP-E immunogen. At an appropriate time after immunization, e.g., when the anti-HsCENP-E antibody titers are highest, antibody-producing cells can be obtained from the subject and used to prepare monoclonal antibodies by standard techniques, such as the hybridoma technique originally described by Kohler and Milstein (1975) Nature 256: 495-497) (see also, Brown et al. (1981) J. Immunol. 127: 539-46; Brown et al. (1980) J. Biol. Chem. 255: 4980-83; Yeh et al. (1976) Proc. Natl. Acad. Sci. USA 76: 2927-31; and Yeh et al. (1982) Int. J. Cancer 29: 269-75), the more recent human B cell hybridoma technique (Kozbor et al. (1983) Immunol Today 4: 72), the EBV-hybridoma technique (Cole et al. (1985), Monoclonal Antibodies and Cancer Therapy, Alan R. Liss, Inc., pp. 77-96) or trioma techniques. A common technique entails fusing an immortal cell line (typically a myeloma) to lymphocytes (typically splenocytes) from a mammal immunized with a HsCENP-E immunogen as described above, followed by screening the culture supernatants of the resulting hybridoma cells to identify a hybridoma producing a monoclonal antibody that binds HsCENP-E.

As the generation of human monoclonal antibodies to HsCENP-E antigen may be difficult with conventional techniques, it may be desirable to create “chimeric antibodies”) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (Cabilly et al., supra; Morrison et al., Proc. Natl. Acad. Sci. USA, 81: 6851-6855, 1984). In general, mouse antibody genes are spliced to human antibody genes to obtain a molecule with appropriate antigen specificity and biological activity. See, e.g., Morrison, S. L. et al. (1984) Proc. Natl. Acad. Sci. 81: 6851-6855; Neuberger, M. S. et al. (1984) Nature 312: 604-608; and Takeda, S. et al. (1985) Nature 314: 452-454.

Other “chimeric” antibodies include hybrid and recombinant antibodies produced by splicing a variable (including hypervariable) domain of an anti-HsCENP-E antibody with a constant domain (e.g “humanized” antibodies), or a light chain with a heavy chain, or a chain from one species with a chain from another species, or fusions with heterologous proteins, regardless of species of origin or immunoglobulin class or subclass designation, as well as antibody fragments (e.g., Fab, F(ab′)₂, and Fv), so long as they exhibit the desired biological activity. See, e.g. Cabilly, et al., U.S. Pat. No. 4,816,567; Mage & Lamoyi, in Monoclonal Antibody Production Techniques and Applications, pp. 79-97 (Marcel Dekker, Inc., New York, 1987).

Humanized monoclonal antibodies, comprising both human and non-human portions, are also encompassed by the invention. Such chimeric and humanized monoclonal antibodies can be produced by recombinant DNA techniques known in the art, for example using methods described in Robinson et al. International Application No. PCT/US86/02269; Akira, et al. European Patent Application 184,187; Taniguchi, M., European Patent Application 171,496; Morrison et al. European Patent Application 173,494; Neuberger et al. PCT International Publication No. WO 86/01533; Cabilly et al. U.S. Pat. No. 4,816,567; Cabilly et al. European Patent Application 125,023; Better et al. (1988) Science 240: 1041-1043; Liu et al. (1987) Proc. Natl. Acad. Sci. USA 84: 3439-3443; Liu et al. (1987) J. Immunol. 139: 3521-3526; Sun et al. (1987) Proc. Natl. Acad. Sci. USA 84: 214-218; Nishimura et al. (1987) Canc. Res. 47: 999-1005; Wood et al. (1985) Nature 314: 446-449; and Shaw et al. (1988) J. Natl. Cancer Inst. 80: 1553-1559); Morrison, S. L. (1985) Science 229: 1202-1207; Oi et al. (1986) BioTechniques 4: 214; Winter U.S. Pat. No. 5,225,539; Jones et al. (1986) Nature 321: 552-525; Verhoeyan et al. (1988) Science 239: 1534; and Beidler et al. (1988) J. Immunol. 141: 4053-4060.

Techniques for the production of single chain antibodies may also be adapted, using methods known in the art, which effectively produce HsCENP-E-specific single chain antibodies. Antibodies with related specificity, but of distinct idiotypic composition, can also be generated by chain shuffling from random combinatorial immunoglobulin libraries. (See, e.g., Burton D. R. (1991) Proc. Natl. Acad. Sci. 88: 10134-10137).

Conventional immunoassays may be employed to identify antibodies having the desired specificity. Such protocols include but are not limited to those described in U.S. Pat. Nos. 4,642,285; 4,376,110; 4,016,043; 3,879,262; 3,852,157; 3,850,752; 3,839,153; 3,791,932; and Harlow and Lane, Antibodies, A Laboratory Manual, Cold Spring Harbor Publications, N.Y. (1988), each incorporated by reference herein.

Immunological procedures useful for in vitro detection of HsCENP-E in a sample are well known. Indeed, numerous protocols for competitive binding or immunoradiometric assays using either polyclonal or monoclonal antibodies with established specificities are known to one skilled in the art. Such immunoassays include, for example, ELISA, Pandex microfluorimetric assay, agglutination assays, flow cytometry, serum diagnostic assays and immunohistochemical staining procedures, which are well known in the art. A typical immunoassay involves the measurement of complex formation between HsCENP-E and its specific antibody. A two-site, monoclonal-based immunoassay utilizing monoclonal antibodies reactive to two non-interfering HsCENP-E epitopes is preferred, but a competitive binding assay may also be employed.

An antibody can be made detectable by various means well known in the art. For example, a detectable marker can be directly or indirectly attached to the antibody. Useful markers include, for example, radionuclides, enzymes, fluorogens, chromogens and chemiluminescent labels.

Methods for assessing the affinity of antibodies for HsCENP-E are also well known. For example, Scatchard analysis in conjunction with radioimmunoassay techniques may be used to assess the affinity of antibodies for HsCENP-E. Affinity is expressed as an association constant, Kₐ, which is defined as the molar concentration of HsCENP-E-antibody complex divided by the molar concentrations of free antigen and free antibody under equilibrium conditions. The Kₐ determined for a preparation of polyclonal antibodies, which are heterogeneous in their affinities for multiple HsCENP-E epitopes, represents the average affinity, or avidity, of the antibodies for HsCENP-E. The Kₐ determined for a preparation of monoclonal antibodies, which are monospecific for a particular HsCENP-E epitope, represents a true measure of affinity. High-affinity antibody preparations with Kₐ ranging from about 10⁹ to 10¹² l/mole are preferred for use in immunoassays in which the HsCENP-E-antibody complex must withstand rigorous manipulations. Low-affinity antibody preparations with Kₐ ranging from about 10⁶ to 10⁷ l/mole are preferred for use in immunopurification and similar procedures which ultimately require dissociation of HsCENP-E, preferably in active form, from the antibody (Catty, D. (1988) Antibodies, Volume I: A Practical Approach, IRL Press, Washington D.C.; Liddell, J. E. and Cryer, A. (1991) A Practical Guide to Monoclonal Antibodies, Johne Wiley & Sons, New York N.Y.).

Kits for generating and screening phage display libraries are commercially available (e.g., the Pharmacia Recombinant Phage Antibody System, Catalog No. 27-9400-01; and the Stratagene SurfZAP.™. Phage Display Kit, Catalog No. 240612). Also see, Ladner et al. U.S. Pat. No. 5,223,409; Kang et al. PCT International Publication No. WO 92/18619; Dower et al. PCT International Publication No. WO 91/17271; Winter et al. PCT International Publication WO 92/20791; Markland et al. PCT International Publication No. WO 92/15679; Breitling et al. PCT International Publication WO 93/01288; McCafferty et al. PCT International Publication No. WO 92/01047; Garrard et al. PCT International Publication No. WO 92/09690; Ladner et al. PCT International Publication No. WO 90/02809; Fuchs et al. (1991) Bio/Technology 9: 1370-1372; Hay et al. (1992) Hum. Antibod. Hybridomas 3: 81-85; Huse et al. (1989) Science 246: 1275-1281; Griffiths et al. (1993) EMBO J. 12: 725-734; Hawkins et al. (1992) J. Mol. Biol. 226: 889-896; Clarkson et al. (1991) Nature 352: 624-628; Gram et al. (1992) Proc. Natl. Acad. Sci. USA 89: 3576-3580; Garrad et al. (1991) Bio/Technology 9: 1373-1377; Hoogenboom et al. (1991) Nuc. Acid Res. 19: 4133-4137; Barbas et al. (1991) Proc. Natl. Acad. Sci. USA 88: 7978-7982; and McCafferty et al. Nature (1990) 348: 552-554.

IV. Uses and Methods of the Invention

The nucleic acid molecules, proteins, protein homologues, and antibodies described herein can be used in one or more of the following methods: a) screening assays; b) predictive medicine e.g., diagnostic assays, and pharmacogenetics); and c) methods of treatment of disorders characterized by excessive cellular proliferation, including cancer.

In the main, the polypeptides of the invention can be used to screen drugs or compounds which modulate activity or expression of HsCENP-E or fragments thereof as well as to treat disorders characterized by excessive production of HsCENP-E or production of a form of HsCENP-E which has increased or aberrant activity compared to the wild type protein. The herein disclosed nucleic acid molecules or sequences derived therefrom can be used in gene therapy to treat HsCENP-E mediated disorders, detect mRNA encoding a human CENP-E or a mutation in a gene encoding HsCENP-E.

A. Screening Assays

In one aspect, the invention provides screening assay(s) for identifying modulators, i.e., candidate or test compounds or agents (e.g., peptides, peptidomimetics, small molecules or other drugs) which bind to HsCENP-E proteins, have an inhibitory effect on, for example, HsCENP-E expression or activity.

In general, assays that can be used to test for modulators of HsCENP-E (target protein) include a variety of in vitro or in vivo assays, e.g., microtubule gliding assays, binding assays such as microtubule binding assays, microtubule depolymerization assays, and ATPase assays (Kodama et al., J. Biochem. 99: 1465-1472 (1986); Stewart et al., Proc. Natl. Acad. Sci. USA 90: 5209-5213 (1993); (Lombillo et al., J. Cell Biol. 128: 107-115 (1995); Vale et al., Cell 42: 39-50 (1985)).

In furtherance of the above, an embodiment of the invention provides a method for identifying a compound that binds to or modulates the activity of a HsCENP-E protein, by providing an indicator composition comprising a HsCENP-E protein having HsCENP-E activity, contacting the indicator composition with a test compound, and determining the effect of the test compound on HsCENP-E activity in the indicator composition to identify a compound that modulates the activity of a HsCENP-E protein.

In yet another aspect, the invention provides a cell-free assay for identifying potential HsCENP-E modulators, comprising contacting a HsCENP-E protein or biologically active portion thereof with a test compound and determining the ability of the test compound to bind to the HsCENP-E protein or biologically active portion thereof. Binding of the test compound to the HsCENP-E protein can be determined either directly or indirectly via known methods.

In another aspect, the invention provides contacting the HsCENP-E protein or biologically active portion thereof with a known compound which binds the polypeptide to form an assay mixture, contacting the assay mixture with a test compound, and determining the ability of the test compound to interact with the HsCENP-E protein, wherein determining the ability of the test compound to interact with the HsCENP-E protein comprises determining the ability of the test compound to preferentially bind to the HsCENP-E protein or biologically active portion thereof as compared to the known compound.

Alternatively, the ability of a compound to modulate activity of a target protein, e.g., HsCENP-E is tested by screening for candidate agents capable of modulating the activity of the target protein. An exemplary assay format comprises the steps of combining a candidate agent with the target protein and determining an alteration in the biological activity of the target protein. In accordance with this embodiment, the candidate agent may bind to the target protein, and as a result thereof, alter its biological or biochemical activity. The methods include both in vitro screening methods and in vivo screening of cells for alterations in cell cycle distribution, cell viability, or for the presence, morphology, activity, distribution, or amount of mitotic spindles.

The test compounds for use in the assays described herein can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12: 145).

There are a number of enzymatic assays known in the art which use ADP as a substrate. For example, kinase reactions such as pyruvate kinases are known. See, Nature 78: 632 (1956) and Mol. Pharmacol. 6: 31 (1970). This is a preferred method in that it allows the regeneration of ATP.

Consequently, in one embodiment, the level of activity of the enzymatic reaction is determined directly. Alternatively, the level of activity of the enzymatic reaction which uses ADP as a substrate is measured indirectly by being coupled to another reaction. Measuring enzymatic reactions by coupling is known in the art. In addition, other protocols are available which utilize phosphate. Example reactions include include a purine nucleoside phosphorylase reaction, which can be measured directly or indirectly.

An exemplary embodiment of the invention proposes detecting ADP or phosphate non-enzymatically, e.g., by binding or reacting the ADP or phosphate with a detectable compound. Suitable examples include phosphomolybdate based assays which involve conversion of free phosphate to a phosphomolybdate complex. One method of quantifying the phosphomolybdate is with malachite green. Alternatively, a fluorescently labeled form of a phosphate binding protein, such as the E. coli phosphate binding protein, can be used to measure phosphate by a shift in its fluorescence.

In yet another embodiment, the present invention provides a method of identifying a candidate agent as a modulator of the activity of a target protein, e.g., HsCENP-E. The method comprises adding a candidate agent to a mixture comprising a target protein which directly or indirectly produces ADP or phosphate, under conditions that normally allow the production of ADP or phosphate. The method further comprises subjecting the mixture to a reaction that uses said ADP or phosphate as a substrate under conditions that normally allow the ADP or phosphate to be utilized and determining the level of activity of the reaction as a measure of the concentration of ADP or phosphate. A change in the level between the presence and absence of the candidate agent indicates a modulator of the target protein. A target protein refers to a molecule with which a selected polypeptide, e.g., HsCENP-E binds or interacts with in nature. In certain instances the target protein can be a HsCENP-E protein.

The phrase “use ADP or phosphate” means that the ADP or phosphate are directly acted upon by detection reagents. In one case, the ADP, for example, can be hydrolyzed or can be phosphorylated. As another example, the phosphate can be added to another compound. As used herein, in each of these cases, ADP or phosphate is acting as a substrate.

Preferably, the target protein either directly or indirectly produces ADP or phosphate and comprises a motor domain. More preferably, the target protein comprises a kinesin superfamily motor protein as described above and most preferably, the target protein comprises HsCENP-E or a fragment thereof.

In another embodiment, modulators of expression of a HsCENP-E protein are identified in a method in which a cell is contacted with a candidate compound and the expression of the selected mRNA or protein (i.e., the mRNA or protein corresponding to a polypeptide or nucleic acid of the invention) in the cell is determined. The level of expression of the selected mRNA or protein in the presence of the candidate compound is compared to the level of expression of the selected mRNA or protein in the absence of the candidate compound. The candidate compound can then be identified as a modulator of expression of the HsCENP-E protein based on this comparison. For example, when expression of the selected mRNA or protein is greater (statistically significantly greater) in the presence of the candidate compound than in its absence, the candidate compound is identified as a stimulator of the selected mRNA or protein expression. Alternatively, when expression of the selected mRNA or protein is less (statistically significantly less) in the presence of the candidate compound than in its absence, the candidate compound is identified as an inhibitor of the selected mRNA or protein expression. The level of the selected mRNA or protein expression in the cells can be determined by methods described herein.

It is also within the scope of this invention to further use a compound (agent) identified as described supra, in an appropriate animal model. For example, an agent identified as described herein (e.g., a HsCENP-E modulating agent, an antisense HsCENP-E nucleic acid molecule, a HsCENP-E-specific antibody, or a HsCENP-E-binding partner) can be used in an animal model to determine the efficacy, toxicity, or side effects of treatment with such a compound.

In an alternative embodiment, the screening assays provided by the invention relate to transgenic mammals whose germ cells and somatic cells contain a nucleotide sequence encoding HsCENP-E protein or a selected portion thereof—SEQ ID NO:2. There are several means by which a sequence encoding, for example, the HsCENP-E may be introduced into a non-human mammalian embryo, some of which are described in, e.g., U.S. Pat. No. 4,736,866, Jaenisch, Science 240-1468-1474 (1988) and Westphal et al., Annu. Rev. Cell Biol. 5: 181-196 (1989), which are incorporated herein by reference. The animal's cells then express the receptor and thus may be used as a convenient model for testing or screening selected agonists or antagonists.

This invention further pertains to novel agents identified by the above-described screening assays and uses thereof for treatments as described herein.

B. Predictive Medicine

Polynucleotides or the polypeptides disclosed herein may also be used as tools in diagnostic assays, and pharmacogenomics as well as for treating individuals afflicted with a HsCENP-E mediated disorder.

In general, the methods described herein can be utilized as diagnostic or prognostic assays to identify subjects having or at risk of developing a disease or disorder associated with aberrant expression or activity of a HsCENP-E protein. For example, the assays described herein, can be utilized to identify a subject having or at risk of developing a disorder associated with aberrant expression or activity of a HsCENP-E protein. Alternatively, the assays can be utilized to identify a subject having or at risk for developing such a disease or disorder.

In accordance with the above, a representative embodiment provides a method in which a test sample is obtained from a subject and a polypeptide or nucleic acid (e.g., mRNA, genomic DNA) of the invention is detected, wherein the presence of the polypeptide or nucleic acid is diagnostic for a subject having or at risk of developing a disease or disorder associated with aberrant expression or activity of the polypeptide. As used herein, a “test sample” refers to a biological sample obtained from a subject of interest. For example, a test sample can be a biological fluid (e.g., serum), cell sample, or tissue.

These and other agents are described in further detail in the following sections.

1. Diagnostic Assays

Accordingly, in one aspect, the invention provides diagnostic assay(s) for identifying the presence or absence of a genetic alteration exemplified by (i) aberrant modification or mutation of a gene encoding a HsCENP-E protein; (ii) mis-regulation of the gene; or (iii) aberrant post-translational modification of a HsCENP-E protein, to thereby determine whether an individual is afflicted with a disease or disorder, or is at risk of developing a disorder, associated with one or more of the above referenced parameters.

For instance, mutations in a gene encoding HsCENP-E can be assayed in a biological sample, and subsequently used for prognostic or predictive purpose to thereby prophylactically treat an individual prior to the onset of a disorder characterized by or associated with aberrant expression or activity of a HsCENP-E protein.

The detection method of the invention can be used to detect mRNA, protein, or genomic DNA in a biological sample in vitro as well as in vivo.

An exemplary method for detecting the presence or absence of a polypeptide or nucleic acid encoding HsCENP-E in a biological sample involves obtaining a biological sample from a test subject and contacting the biological sample with a compound or an agent capable of detecting a polypeptide or nucleic acid (e.g., mRNA, genomic DNA) such that the presence of a polypeptide or nucleic acid is detected in the biological sample.

A preferred agent for detecting mRNA or genomic DNA encoding a HsCENP-E protein is a labeled nucleic acid probe capable of hybridizing to mRNA or genomic DNA encoding a HsCENP-E protein. The nucleic acid probe can be, for example, a full-length cDNA, such as the nucleic acid of SEQ ID NO:1, or a portion derived therefrom, such as an oligonucleotide of at least 15, 30, 50, 100, 250 or 500 nucleotides in length and sufficient to specifically hybridize under moderately stringent conditions to a mRNA or genomic DNA encoding a HsCENP-E protein. Other suitable probes for use in the diagnostic assays of the invention are described herein. In certain embodiments, polynucleotides or longer fragments derived from the polynucleotide sequence described herein may be used in various diagnostic assays to detect the presence of HsCENP-E mediated disorders. Nucleic acids for diagnosis may be obtained from body fluids or cell extracts which are known to express HsCENP-E.

The probes may be labeled by standard methods and added to a fluid or tissue sample from a patient under conditions suitable for the formation of hybridization complexes. After a suitable incubation period, the sample is washed and the signal is quantitated and compared with a standard value. If the amount of signal in the patient sample is significantly altered in comparison to a control sample then the presence of altered levels of nucleotide sequences encoding HsCENP-E in the sample indicates the presence of the associated disorder. Such assays may also be used to evaluate the efficacy of a particular therapeutic treatment regimen in animal studies, in clinical trials, or to monitor the treatment of an individual patient.

In one aspect, hybridization with PCR probes which are capable of detecting polynucleotide sequences, including genomic sequences encoding a human CENP-E protein or closely related molecules may be used to identify nucleic acid sequences which encode a HsCENP-E protein. The specificity of the probe, whether it is made from a highly specific region, e.g., the 5′ regulatory region, or from a less specific region, e.g., a conserved motif, and the stringency of the hybridization or amplification (maximal, high, intermediate, or low), will determine whether the probe identifies only naturally occurring sequences encoding HsCENP-E, allelic variants, or related sequences.

Probes may also be used for the detection of related sequences, and should preferably have at least 50% sequence identity to any of the HsCENP-E encoding sequences. The hybridization probes of the subject invention may be DNA or RNA and may be derived from the sequence of SEQ ID NO:1 or from genomic sequences including promoters, enhancers, and introns of the HsCENP-E gene.

Means for producing specific hybridization probes for DNAs encoding HsCENP-E include cloning polynucleotide sequences encoding HsCENP-E or HsCENP-E derivatives into vectors for the production of mRNA probes. Such vectors are known in the art, are commercially available, and may be used to synthesize RNA probes in vitro by means of the addition of the appropriate RNA polymerases and the appropriate labeled nucleotides. Hybridization probes may be labeled by a variety of reporter groups, for example, by radionuclides such as ³²P or ³⁵S, or by enzymatic labels, such as alkaline phosphatase coupled to the probe via avidin/biotin coupling systems, and the like.

In order to provide a basis for the diagnosis of a disorder associated with expression of HsCENP-E, a normal or standard profile for expression is established. This may be accomplished by combining body fluids or cell extracts taken from normal subjects, either animal or human, with a sequence, or a fragment thereof, encoding HsCENP-E, under conditions suitable for hybridization or amplification. Standard hybridization may be quantified by comparing the values obtained from normal subjects with values from an experiment in which a known amount of a substantially purified polynucleotide is used. Standard values obtained in this manner may be compared with values obtained from samples from patients who are symptomatic for a disorder. Deviation from standard values is used to establish the presence of a disorder.

A general method corresponding to the above entails obtaining a control biological sample from a control subject, contacting the control sample with a compound or agent capable of detecting a HsCENP-E protein or mRNA or genomic DNA encoding a HsCENP-E protein, such that the presence of the protein or mRNA or genomic DNA encoding the protein is detected in the biological sample, and comparing the presence of the polypeptide or mRNA or genomic DNA encoding the HsCENP-E protein in the control sample with the presence of the protein or mRNA or genomic DNA encoding the polypeptide in the test sample. Once the presence of a disorder is established and a treatment protocol is initiated, hybridization assays may be repeated on a regular basis to determine if the level of expression in the patient begins to approximate that which is observed in the normal subject. The results obtained from successive assays may be used to show the efficacy of treatment over a period ranging from several days to months.

Polynucleotides encoding the amino acid of SEQ ID NO:2 or biologically equivalent variants thereof or having the nucleotide sequence as set forth in SEQ ID NO: 1 including sequences derived therefrom may be used to detect a mutated form of an HsCENP-E gene associated with a dysfunction that would eventually provide a diagnostic tool that can add to or define a diagnosis of a disease or susceptibility to a disease which results from the abnormal or altered expression of HsCENP-E.

A mutation(s) can be detected by ascertaining the existence of at least one of: 1) a deletion of one or more nucleotides from the gene; 2) an addition of one or more nucleotides to the gene; 3) a substitution of one or more nucleotides of the gene; 4) a chromosomal rearrangement of the gene; 5) an alteration in the level of a messenger RNA transcript of the gene; 6) an aberrant modification of the gene, such as of the methylation pattern of the genomic DNA; 7) the presence of a non-wild type splicing pattern of a messenger RNA transcript of the gene; 8) a non-wild type level of a the protein encoded by the gene; 9) an allelic loss of the gene; and 10) an inappropriate post-translational modification of the protein encoded by the gene. As described herein, there are a large number of assay techniques known in the art which can be used for detecting lesions in a gene.

Consequently, the polynucleotides encoding HsCENP-E may be used to detect and quantitate HsCENP-E gene expression in biopsied tissues in which expression of HsCENP-E may be correlated with disease. The diagnostic assay may be used to determine absence, presence, and excess expression of HsCENP-E, and to monitor regulation of HsCENP-E levels during therapeutic intervention. Methods for quantifying the expression of HsCENP-E include radiolabeling or biotinylating nucleotides, coamplification of a control nucleic acid, and interpolating results from standard curves (See, e.g., Melby, P. C. et al. (1993) J. Imunol. Methods 159: 235-244; Duplaa, C. et al. (1993) Anal. Biochem. 229-236). The speed of quantitation of multiple samples may be accelerated by running the assay in an ELISA format where the oligomer of interest is presented in various dilutions and a spectrophotometric or calorimetric response gives rapid quantitation.

With respect to cancer, the presence of an abnormal amount of transcript, preferably, an increased level of a transcript encoding HsCENP-E protein in biopsied tissue from an individual may indicate a predisposition for the development of the disease, or may provide a means for detecting the disease prior to the appearance of actual clinical symptoms. A more definitive diagnosis of this type may allow health professionals to employ preventative measures or aggressive treatment earlier thereby preventing the development or further progression of the cancer.

For instance, point mutations can be identified by hybridizing amplified DNA to labeled HsCENP-E nucleotide sequences. Perfectly matched sequences can be distinguished from mismatched duplexes by RNase digestion or by differences in melting temperatures. DNA sequence differences may also be detected by alterations in electrophoretic mobility of DNA fragments in gels, with or without denaturing agents, or by direct DNA sequencing. See, e.g., Myers et al., Science (1985) 230: 1242. Sequence changes at specific locations may also be revealed by nuclease protection assays, such as RNase and S 1 protection or the chemical cleavage method. See Cotton et al., Proc Natl Acad Sci USA (1985) 85: 4397-4401.

In preferred embodiments, the methods include detecting, in a sample of cells from the subject, the presence or absence of a mutation characterized by at least one of an alteration affecting the integrity of a gene encoding the HsCENP-E protein, or the mis-expression of the gene encoding the HsCENP-E protein.

In certain embodiments, detection of the mutation involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241: 1077-1080; and Nakazawa et al. (1994) Proc. Natl. Acad. Sci. USA 91: 360-364), the latter of which can be particularly useful for detecting point mutations in a gene (see, e.g., Abravaya et al. (1995) Nucleic Acids Res. 23: 675-682). This method can include the steps of collecting a sample of cells from a patient, isolating nucleic acid (e.g., genomic, mRNA or both) from the cells of the sample, contacting the nucleic acid sample with one or more primers which specifically hybridize to the selected gene under conditions such that hybridization and amplification of the gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein. Other amplification methods are well known.

In an alternative embodiment, mutations in a selected gene e.g., HsCENP-E encoding gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, e.g., U.S. Pat. No. 5,498,531) can be used to score for the presence of specific mutations by development or loss of a ribozyme cleavage site.

In other embodiments, genetic mutations can be identified by hybridizing a sample and control nucleic acids, e.g., DNA or RNA, to high density arrays containing hundreds or thousands of polynucleotide probes (Cronin et al. (1996) Human Mutation 7: 244-255; Kozal et al. (1996) Nature Medicine 2: 753-759). For example, genetic mutations can be identified in two-dimensional arrays containing light-generated DNA probes as described in Cronin et al., supra. Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the selected gene and detect mutations by comparing the sequence of the sample nucleic acids with the corresponding wild-type (control) sequence. Examples of sequencing reactions include those based on techniques developed by Maxim and Gilbert ((1977) Proc. Natl. Acad. Sci. USA 74: 560) or Sanger ((1977) Proc. Natl. Acad. Sci. USA 74: 5463). It is also contemplated that any of a variety of automated sequencing procedures can be utilized when performing the diagnostic assays ((1995) Bio/Techniques 19: 448), including sequencing by mass spectrometry (see, e.g., PCT Publication No. WO 94/16101; Cohen et al. (1996) Adv. Chromatogr. 36: 127-162; and Griffin et al. (1993) Appl. Biochem. Biotechnol. 38: 147-159).

Other methods for detecting mutations in a HsCENP-E gene include methods in which protection from cleavage agents is used to detect mismatched bases in RNA/RNA or RNA/DNA heteroduplexes (Myers et al. (1985) Science 230: 1242). In general, the technique of a mismatch cleavage entails providing heteroduplexes formed by hybridizing (labeled) RNA or DNA containing the wild-type sequence with potentially mutant RNA or DNA obtained from a tissue sample. The double-stranded duplexes are treated with an agent which cleaves single-stranded regions of the duplex such as which will exist due to basepair mismatches between the control and sample strands. RNA/DNA duplexes can be treated with RNase to digest mismatched regions, and DNA/DNA hybrids can be treated with S1 nuclease to digest mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, e.g., Cotton et al. (1988) Proc. Natl. Acad. Sci. USA 85: 4397; Saleeba et al. (1992) Methods Enzymol. 217: 286-295. In a preferred embodiment, the control DNA or RNA can be labeled for detection. See, also U.S. Pat. No. 5,459,039.

In other embodiments, alterations in electrophoretic mobility will be used to identify mutations in genes. For example, single strand conformation polymorphism (SSCP) may be used to detect differences in electrophoretic mobility between mutant and wild type nucleic acids (Orita et al. (1989) Proc. Natl. Acad. Sci. USA 86: 2766; see also Cotton (1993) Mutat. Res. 285: 125-144; Hayashi (1992) Genet. Anal. Tech. Appl. 9: 73-79). Single-stranded DNA fragments of sample and control nucleic acids will be denatured and allowed to renature. The secondary structure of single-stranded nucleic acids varies according to sequence, and the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet. 7: 5).

In yet another embodiment, the movement of mutant or wild-type fragments in polyacrylamide gels containing a gradient of denaturant is assayed using denaturing gradient gel electrophoresis (DGGE) (Myers et al. (1985) Nature 313: 495). When DGGE is used as the method of analysis, DNA will be modified to insure that it does not completely denature, for example by adding a 'GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys. Chem. 265: 12753).

Other suitable protocols for detecting point mutations include, but are not limited to, selective oligonucleotide hybridization, selective amplification, or selective primer extension. See for example Saiki et al. (1986) Nature 324: 163); Saiki et al. (1989) Proc. Natl. Acad. Sci. USA 86: 6230).

By means of having the HsCENP-E molecule and antibodies thereto, a variety of diagnostic assays are provided. Antibodies can be used for treatment or to identify the presence of HsCENP-E protein having the sequence identity characteristics as described herein. Additionally, antibodies can be used to identify modulators of the interaction between the antibody and HsCENP-E protein as further described below.

While the following discussion is directed toward the use of antibodies in the use of binding assays, it is understood that the same general assay formats such as those described for “non-competitive” or “competitive” assays can be used with any compound which binds to HsCENP-E protein such as microtubules etc. For a review of immunological and immunoassay procedures, see Basic and Clinical Immunology (Stites & Terr eds., 7th ed. 1991). Moreover, the immunoassays of the present invention can be performed in any of several configurations, which are reviewed extensively in Enzyme Immunoassay (Maggio ed., 1980); and Harlow & Lane, supra.

Preferably, HsCENP-E protein is detected and/or quantified using any of a number of well recognized immunological binding assays (see, e.g., U.S. Pat. Nos. 4,366,241; 4,376,110; 4,517,288; and 4,837,168). For a review of the general immunoassays, see also Methods in Cell Biology Volume 37: Antibodies in Cell Biology (Asai, ed. 1993); Basic and Clinical Immunology (Stites & Terr, eds., 7th ed. 1991).

Immunological binding assays (or immunoassays) typically use an antibody that specifically binds to a protein or antigen of choice, vis-à-vis the HsCENP-E protein or antigenically active fragment thereof. The antibody, e.g., anti-HsCENP-E protein may be produced by any of a number of means well known to those of skill in the art and as described above.

Thus, a representative embodiment of the invention proposes utilizing an antibody which specifically binds a HsCENP-E protein for the diagnosis of disorders characterized by expression of HsCENP-E, or in assays to monitor patients being treated with HsCENP-E or agonists, antagonists, or inhibitors of HsCENP-E. Preferably, the antibody has a binding specificity for the gene product of SEQ ID NO:1 or the polypeptide of SEQ ID NO:2 or an antigenically active fragment thereof.

A variety of protocols exist that are suitable for measuring HsCENP-E. Representative examples include ELISAs, RIAs, and FACS, each of which provides a basis for diagnosing altered or abnormal levels of HsCENP-E expression.

Normal or standard values for HsCENP-E expression can be established by combining body fluids or cell extracts taken from normal mammalian subjects, preferably human, with antibody to HsCENP-E under conditions favoring the formation of a complex therewith. The amount of standard complex formation may be quantitated by various methods. Quantities of HsCENP-E expressed in a subject, control, and disease samples from biopsied tissues can thereafter be compared with the standard values. Deviation between standard and subject values, in turn, provides the parameters for diagnosing disease.

In one assay format, a HsCENP-E protein is identified and/or quantified by using labeled antibodies, preferably monoclonal antibodies which are reacted with body tissue known to express high levels HsCENP-E and determining the specific binding thereto, the assay typically being performed under conditions conducive to immune complex formation. Unlabeled primary antibody can be used in combination with labels that are reactive with primary antibody to detect the receptor.

Immunoassays also often use a labeling agent to specifically bind to and label the complex formed by the antibody and antigen. The labeling agent may itself be one of the moieties comprising the antibody/antigen complex. Thus, the labeling agent may be a labeled HsCENP-E protein polypeptide or a labeled anti-HsCENP-E protein antibody. Alternatively, the labeling agent may be a third moiety, such a secondary antibody, that specifically binds to the antibody/HsCENP-E protein complex (a secondary antibody is typically specific to antibodies of the species from which the first antibody is derived). Other proteins capable of specifically binding immunoglobulin constant regions, such as protein A or protein G may also be used as the label agent. These proteins exhibit a strong non-immunogenic reactivity with immunoglobulin constant regions from a variety of species (see generally Kronval et al., J. Immunol. 111: 1401-1406 (1973); Akerstrom et al., J. Immunol. 135: 2589-2542 (1985)). The labeling agent can be modified with a detectable moiety, such as biotin, to which another molecule can specifically bind, such as streptavidin. A variety of detectable moieties are well known to those skilled in the art.

Non-Competitive Assay Formats—Immunoassays for detecting HsCENP-E protein in samples may be either competitive or noncompetitive. Noncompetitive immunoassays are assays in which the amount of antigen is directly measured. In one preferred “sandwich” assay, for example, the anti-HsCENP-E protein antibodies can be bound directly to a solid substrate on which they are immobilized. These immobilized antibodies then capture HsCENP-E protein present in the test sample. HsCENP-E protein is thus immobilized is then bound by a labeling agent, such as a second HsCENP-E protein antibody bearing a label. Alternatively, the second antibody may lack a label, but it may, in turn, be bound by a labeled third antibody specific to antibodies of the species from which the second antibody is derived. The second or third antibody is typically modified with a detectable moiety, such as biotin, to which another molecule specifically binds, e.g., streptavidin, to provide a detectable moiety.

In an alternative format, e.g., competitive assay formats, the amount of HsCENP-E protein present in the sample is measured indirectly by measuring the amount of a known, added (exogenous) HsCENP-E protein displaced (competed away) from an anti-HsCENP-E protein antibody by the unknown HsCENP-E protein present in a sample. In one competitive assay, a known amount of HsCENP-E protein is added to a sample and the sample is then contacted with an antibody that specifically binds to HsCENP-E protein. The amount of exogenous HsCENP-E protein bound to the antibody is inversely proportional to the concentration of HsCENP-E protein present in the sample. In a particularly preferred embodiment, the antibody is immobilized on a solid substrate. The amount of HsCENP-E protein bound to the antibody may be determined either by measuring the amount of HsCENP-E protein present in a HsCENP-E protein/antibody complex, or alternatively by measuring the amount of remaining uncomplexed protein. The amount of HsCENP-E protein may be detected by providing a labeled HsCENP-E protein molecule.

A hapten inhibition assay is another preferred competitive assay. In this assay the known HsCENP-E protein, is immobilized on a solid substrate. A known amount of anti-HsCENP-E protein antibody is added to the sample, and the sample is then contacted with the HsCENP-E protein. The amount of anti-HsCENP-E protein antibody bound to the known immobilized HsCENP-E protein is inversely proportional to the amount of HsCENP-E protein present in the sample. Again, the amount of immobilized antibody may be detected by detecting either the immobilized fraction of antibody or the fraction of the antibody that remains in solution. Detection may be direct where the antibody is labeled or indirect by the subsequent addition of a labeled moiety that specifically binds to the antibody as described above.

A western blot analysis can be used to detect and quantify the presence of HsCENP-E protein in the sample. The technique generally comprises separating sample proteins by gel electrophoresis on the basis of molecular weight, transferring the separated proteins to a suitable solid support, (such as a nitrocellulose filter, a nylon filter, or derivatized nylon filter), and incubating the sample with the antibodies that specifically bind HsCENP-E protein. The anti-HsCENP-E protein antibodies specifically bind to the HsCENP-E protein on the solid support. These antibodies may be directly labeled or alternatively may be subsequently detected using labeled antibodies (e.g., labeled sheep anti-mouse antibodies) that specifically bind to the anti-HsCENP-E protein antibodies. Other assay formats include liposome immunoassays (LIA), which use liposomes designed to bind specific molecules (e.g., antibodies) and release encapsulated reagents or markers. The released chemicals are then detected according to standard techniques (see Monroe et al., Amer. Clin. Prod. Rev. 5: 34-41 (1986)).

Reduction of non-specific binding—It will be appreciated that it is often desirable to minimize non-specific binding in immunoassays. Particularly, where the assay involves an antigen or antibody immobilized on a solid substrate it is desirable to minimize the amount of non-specific binding to the substrate. Means of reducing such non-specific binding are well known to those of skill in the art. Typically, this technique involves coating the substrate with a proteinaceous composition. In particular, protein compositions such as bovine serum albumin (BSA), nonfat powdered milk, and gelatin are widely used with powdered milk being most preferred.

The term “labeled,” with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. The particular label or detectable group used in the assays disclosed herein is not critical, so long as it does not significantly interfere with the specific binding of the antibody used in the particular assay. The detectable group can be any material having a detectable physical or chemical property. Such detectable labels have been well-developed in the field of immunoassays and, in general, most any label useful in such methods can be applied to the present invention.

“Biological sample” as used herein is a sample of biological tissue or fluid that contains a target protein, e.g. HsCENP-E protein or a fragment thereof or nucleic acid encoding a target protein or a fragment thereof, e.g., HsCENP-E. Biological samples may also include sections of tissues such as frozen sections taken for histological purposes. The term is intended to include tissues, cells and biological fluids isolated from a subject, as well as tissues, cells and fluids present within a subject.

The invention also encompasses kits for detecting the presence of a polypeptide or nucleic acid of the invention in a biological sample (a test sample). Such kits can be used to determine if a subject is suffering from or is at increased risk of developing a disorder associated with aberrant expression of a HsCENP-E protein (e.g., HsCENP-E mediated or associated cancers). For example, the kit can comprise a labeled compound or agent capable of detecting the polypeptide or mRNA encoding the polypeptide in a biological sample and means for determining the amount of the HsCENP-E protein or mRNA in the sample (e.g., an antibody which binds the polypeptide or an oligonucleotide probe which binds to DNA or mRNA encoding the polypeptide). Kits may also include instruction for observing that the tested subject is suffering from or is at risk of developing a disorder associated with aberrant expression of the polypeptide if the amount of the polypeptide or mRNA encoding the polypeptide is above or below a normal level.

For antibody-based kits, the kit may comprise, for example: (1) a first antibody (e.g., attached to a solid support) which binds to a HsCENP-E protein; and, optionally, (2) a second, different antibody which binds to either the polypeptide or the first antibody and is conjugated to a detectable agent.

For oligonucleotide-based kits, the kit may comprise, for example: (1) an oligonucleotide, e.g., a detectably labeled oligonucleotide, which hybridizes to a nucleic acid sequence encoding a HsCENP-E protein or (2) a pair of primers useful for amplifying a nucleic acid molecule encoding a HsCENP-E protein.

The kit may also comprise, e.g., a buffering agent, a preservative, or a protein stabilizing agent. The kit may also comprise components necessary for detecting the detectable agent (e.g., an enzyme or a substrate). The kit may also contain a control sample or a series of control samples which can be assayed and compared to the test sample contained. Each component of the kit is usually enclosed within an individual container and all of the various containers are within a single package along with instructions for observing whether the tested subject is suffering from or is at risk of developing a disorder associated with aberrant expression of the polypeptide.

In one embodiment, for example, the diagnostic nucleic acids are derived from SEQ ID NO:1. The contemplated diagnostic systems are useful for assaying for the presence or absence of nucleic acid encoding the invention protein in either genomic DNA or in transcribed nucleic acid (such as mRNA or cDNA) encoding the invention protein.

A suitable diagnostic system includes at least one nucleic acid molecule of the invention or a fragment derived therefrom, preferably two or more invention nucleic acids, as a separately packaged chemical reagent(s) in an amount sufficient for at least one assay. Instructions for use of the packaged reagent are also typically included. Those of skill in the art can readily incorporate invention nucleic probes and/or primers into kit form in combination with appropriate buffers and solutions for the practice of the invention methods as described herein.

“Invention protein” or “protein of the invention” refers to the gene product of SEQ ID NO:1 or variants or fragments thereof, all of which encode the amino acid sequence of SEQ ID NO:2 or a biologically active HsCENP-E.

2. Pharmacogenomics

The invention further provides methods for expression of a nucleic acid or HsCENP-E protein or activity of a HsCENP-E protein in an individual to thereby select appropriate therapeutic or prophylactic agents for that individual (referred to herein as “pharmacogenomics”).

“Pharmacogenomics”, as used herein, refers to the application of genomics technologies such as gene sequencing, statistical genetics, and gene expression analysis to drugs in clinical development and on the market. More specifically, the term refers the study of how a patient's genes determine his or her response to a drug (e.g., a patient's “drug response phenotype”, or “drug response genotype”). See, e.g., Linder (1997) Clin. Chem. 43(2): 254-266.

Thus, an aspect of the invention provides methods for tailoring an individual's prophylactic or therapeutic treatment with either the HsCENP-E molecules of the present invention or HsCENP-E modulators according to that individual's drug response genotype.

Consequently, modulators which inhibit the activity or expression of a HsCENP-E protein as identified by any one of the screening assays described herein can be administered to individuals to treat (prophylactically or therapeutically) disorders associated with aberrant activity of the polypeptide. In conjunction with such treatment, the pharmacogenomics (i.e., the study of the relationship between an individual's genotype and that individual's response to a foreign compound or drug) of the individual may be considered. As such, the proposed methods of the invention will effectively allow a clinician or physician to target prophylactic or therapeutic treatments to patients who will most benefit from the treatment and to avoid treatment of patients who will experience toxic drug-related side effects.

Such pharmacogenomics can further be used to determine appropriate dosages and therapeutic regimens. Consequently, the activity of a HsCENP-E protein, expression of a nucleic acid encoding the polypeptide, or mutation content of a gene encoding the polypeptide in an individual can be determined to thereby select appropriate agent(s) for therapeutic or prophylactic treatment of the individual.

For example, the effectiveness of an agent identified herein, that has been implicated in decreasing HsCENP-E gene expression, HsCENP-E protein levels or its protein activity, can be monitored in clinical trials of subjects exhibiting increased gene expression, protein levels, or protein activity.

For example, and not by way of limitation, genes, including those of the invention, vis-a-vis SEQ ID NO:1 or encoding a protein of SEQ ID NO:2 that are modulated in cells by treatment with an agent (e.g., compound, drug or small molecule) which modulates activity or expression of a HsCENP-E polypeptide (e.g., as identified in a screening assay described herein) can be identified.

Thus, to study the effect of agents (e.g., anti-mitotic agents) on a specific HsCENP-E mediated or associated cancers, e.g., those characterized by hyper-proliferative cells including those resistant to Taxol or other drug-resistant tumors, for example, in a clinical trial, cells can be isolated and RNA prepared and analyzed for the levels of expression of a gene of the invention and other genes implicated in the disorder. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of a gene of the invention or other genes. In this way, the gene expression pattern can serve as a marker, indicative of the physiological response of the cells to the agent. Accordingly, this response state may be determined before, and at various points during, treatment of the individual with the agent.

Consequently, an exemplary embodiment of the invention provides a method for monitoring the effectiveness of treatment of a subject with an agent (e.g., an agonist, antagonist, peptidomimetic, protein, peptide, nucleic acid, small molecule, or other drug candidate identified by the screening assays described herein) comprising the steps of (i) obtaining a pre-administration sample from a subject prior to administration of the agent; (ii) detecting the level of the polypeptide or nucleic acid of the invention in the pre-administration sample; (iii) obtaining one or more post-administration samples from the subject; (iv) detecting the level the of the polypeptide or nucleic acid of the invention in the post-administration samples; (v) comparing the level of the polypeptide or nucleic acid of the invention in the pre-administration sample with the level of the polypeptide or nucleic acid of the invention in the post-administration sample or samples; and (vi) altering the administration of the agent to the subject accordingly. For example, increased administration of the agent may be desirable to reduce expression or activity of the polypeptide, i.e., to increase the effectiveness of the agent.

C. Methods of Treatment

The present invention also provides for both prophylactic and therapeutic methods of treating a subject at risk of (or susceptible to) a disorder or having a disorder associated with aberrant HsCENP-E expression or activity. With regards to both prophylactic and therapeutic methods of treatment, such treatments may be specifically tailored or modified, based on knowledge obtained from the field of pharmacogenomics.

1. Prophylactic Methods

In accordance with the above, the invention provides a method for preventing in a subject, a disease or condition associated with an aberrant HsCENP-E expression or activity, by administering to the subject a HsCENP-E or an agent which modulates HsCENP-E expression or at least one HsCENP-E activity. Subjects at risk for a disease which is caused or contributed to by aberrant HsCENP-E expression or activity can be identified by, for example, any or a combination of diagnostic or prognostic assays as described herein. Administration of a prophylactic agent can occur prior to the manifestation of symptoms characteristic of the HsCENP-E aberrancy, such that a disease or disorder is prevented or, alternatively, delayed in its progression. Depending on the type of HsCENP-E aberrancy, for example, a HsCENP-E agonist or HsCENP-E antagonist agent can be used for treating the subject. The appropriate agent can be determined based on screening assays described herein.

2. Therapeutic Methods

It is well known that chemical and structural similarity, e.g., in the context of sequences and motifs, exists between regions of HsCENP-E and the motor domain of kinesin. As well, the prior art is replete with teaching implicating the expression of HsCENP-E in disease states attended by hyper-proliferating cells such as, for example, cancer. Therefore, in the treatment of disease states characterized by increased HsCENP-E activity, it is desirable to decrease the expression or activity of HsCENP-E. Alternatively, in the treatment of pathological conditions attended with decreased HsCENP-E activity, it is desirable to provide the protein or to increase the expression of HsCENP-E.

Generally, inhibition of HsCENP-E activity is desirable in situations in which HsCENP-E is abnormally upregulated and/or in which decreased HsCENP-E activity is likely to have a beneficial effect. Preferably, the agent inhibits one or more HsCENP-E activities. Examples of such inhibitory agents include antisense HsCENP-E nucleic acid molecules, anti-HsCENP-E antibodies, and HsCENP-E inhibitors. These modulatory methods can be performed in vitro (e.g., by culturing the cell with the agent) or, alternatively, in vivo (e.g., by administering the agent to a subject).

Increased expression of a HsCENP-E protein can be treated by administering an antagonist of HsCENP-E to the subject in an amount effective to treat or prevent a disorder associated with increased expression or activity of HsCENP-E. Such disorders may include, but are not limited to, those discussed above.

The antibody which specifically binds HsCENP-E may be used directly as an antagonist or indirectly as a targeting or delivery mechanism for bringing a pharmaceutical agent to cells or tissue which express HsCENP-E. A preferred embodiment of the present invention involves a method for treatment of a HsCENP-E associated disease or disorder which includes the step of administering a therapeutically effective amount of a HsCENP-E antibody to a subject.

As defined herein, a therapeutically effective amount of antibody (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of an antibody can include a single treatment or, preferably, can include a series of treatments. In a preferred example, a subject is treated with antibody in the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of antibody used for treatment may increase or decrease over the course of a particular treatment. Changes in dosage may result from the results of diagnostic assays as described herein.

As noted, supra, expression of a gene encoding endogenous human CENP-E protein can be inhibited using expression blocking techniques.

Consequently, antisense-constructs—DNA or RNA that are taken up by suitable host cells but are not expressed and instead block the expression of certain genes in vivo are also contemplated by the methods of the invention.

Known such techniques involve the use of antisense sequences, either internally generated or separately administered. See, for example, O'Connor, J Neurochem (1991) 56: 560 in Oligodeoxynucleotides as Antisense Inhibitors of Gene Expression, CRC Press, Boca Raton, Fla. (1988). Alternatively, polynucleotides which form triple helices with the gene can be supplied. See, for example, Lee et al., Nucleic Acids Res (1979) 6: 3073; Cooney et al., Science (1988) 241: 456; Dervan et al., Science (1991) 251: 1360. These oligomers can be administered per se or the relevant oligomers can be expressed in vivo.

The prior art is replete with teachings showing that short antisense oligonucleotides can be imported into cells where they act as inhibitors, despite their low intracellular concentrations caused by their restricted uptake by the cell membrane. (Zamecnik et al., Proc. Natl. Acad. Sci. USA 83, 4143-4146 [1986]). The polynucleotides can be modified to enhance the uptake, e.g., by substituting their negatively charged phosphodiester groups with uncharged groups.

In furtherance of the above, an aspect of the invention is drawn to the introduction of antisense constructs prepared through the use of antisense technology, which may be used to control gene expression through triple-helix formation or antisense DNA or RNA, both of which methods are based on binding of a polynucleotide to DNA or RNA.

For the purpose of this invention, the intended objective with respect to DNA of a cell is to interfere with its replication and transcription. Likewise, with respect to RNA, it is an object of the invention to interfere with all vital functions such as, for example, translocation of the RNA to the site of protein translation, translation of protein from the RNA, splicing of the RNA to yield one or more mRNA species, and catalytic activity which may be engaged in or facilitated by the RNA. The overall effect of the proposed effect within the context of the above embodiment—is the modulation of the expression of the target nucleic acid in the host cell. As used within the context of this embodiment, a “target nucleic acid” is intended to cover DNA or RNA whose replication or transcription is intended to be inhibited. As such, “modulation” with respect to the above referenced embodiment means a decrease or complete inhibition in the expression of the “target nucleic acid”.

The technique for the above embodiment details engineering an oligonucleotide so as to be complementary to a region of the gene involved in transcription of the mRNA to be targeted. The antisense RNA polynucleotide is thereafter introduced into suspected host cells in accordance with a method of the invention and under conditions favoring its uptake and subsequent expression whereby it hybridizes to mRNA in vivo and blocks translation of the mRNA molecules into proteins (triple helix—see Lee et al., Nucl. Acids Res., 6: 3073 (1979); Cooney et al, Science, 241: 456 (1988); and Oligodeoxynucleotides as Antisense Inhibitors of Gene Expression, CRC Press, Boca Raton, Fla. (1988).

As such, an embodiment of the invention proposes administering a vector expressing the complement of the polynucleotide encoding HsCENP-E to a subject to treat or prevent a disorder associated with increased expression or activity of HsCENP-E including, but not limited to, those described above.

For practicing the above embodiments, the antisense DNA sequences may use natural nucleotides or unnatural nucleotide mimics known in the art.

In another embodiment of the invention, the complement of the polynucleotide encoding HsCENP-E may be used in situations in which it would be desirable to block the transcription of the mRNA. In particular, cells may be transformed with sequences complementary to polynucleotides encoding HsCENP-E. Thus, complementary molecules or fragments may be used to modulate HsCENP-E activity, or to achieve regulation of gene function. Such technology is now well known in the art, and sense or antisense polynucleotides or larger fragments can be designed from various locations along the coding or control regions of sequences encoding HsCENP-E.

Expression vectors derived from retroviruses, adenoviruses, or herpes or vaccinia viruses, or from various bacterial plasmids, may be used for delivery of nucleotide sequences to the targeted organ, tissue, or cell population. Methods which are well known to those skilled in the art can be used to construct vectors to express nucleic acid sequences complementary to the polynucleotides encoding HsCENP-E. (See, e.g., Sambrook, supra; Ausubel, 1995, supra.).

Genes encoding HsCENP-E can be turned off by transforming a cell or tissue with expression vectors which express high levels of a polynucleotide, or fragment thereof, encoding HsCENP-E. Such constructs may be used to introduce untranslatable sense or antisense sequences into a cell. Even in the absence of integration into the DNA, such vectors may continue to transcribe RNA molecules until they are disabled by endogenous nucleases. Transient expression may last for a month or more with a non-replicating vector, and may last even longer if appropriate replication elements are part of the vector system.

As mentioned above, modifications of gene expression can be obtained by designing complementary sequences or antisense molecules (DNA, RNA, or PNA) to the control, 5′, or regulatory regions of the gene encoding HsCENP-E. Polynucleotides derived from the transcription initiation site, e.g., between about positions −10 and +10 from the start site, are preferred. Similarly, inhibition can be achieved using triple helix base-pairing methodology. Triple helix pairing is useful because it causes inhibition of the ability of the double helix to open sufficiently for the binding of polymerases, transcription factors, or regulatory molecules. Recent therapeutic advances using triplex DNA have been described in the literature. (See, erg., Gee, J. E. et al. (1994) in Huber, B. E. and B. I. Carr, Molecular and Immunologic Approaches, Futura Publishing, Mt. Kisco N.Y., pp. 163-177). A complementary sequence or antisense molecule may also be designed to block translation of mRNA by preventing the transcript from binding to ribosomes.

Ribozymes, enzymatic RNA molecules, may also be used to catalyze the specific cleavage of RNA. The mechanism of ribozyme action involves sequence-specific hybridization of the ribozyme molecule to complementary target RNA, followed by endonucleolytic cleavage. For example, engineered hammerhead motif ribozyme molecules may specifically and efficiently catalyze endonucleolytic cleavage of sequences encoding HsCENP-E.

Specific ribozyme cleavage sites within any potential RNA target are initially identified by scanning the target molecule for ribozyme cleavage sites, including the following sequences: GUA, GUU, and GUC. Once identified, short RNA sequences of between 15 and 20 ribonucleotides, corresponding to the region of the target gene containing the cleavage site, may be evaluated for secondary structural features which may render the oligonucleotide inoperable. The suitability of candidate targets may also be evaluated by testing accessibility to hybridization with complementary polynucleotides using ribonuclease protection assays.

Complementary ribonucleic acid molecules and ribozymes of the invention may be prepared by any method known in the art for the synthesis of nucleic acid molecules. These include techniques for chemically synthesizing polynucleotides such as solid phase phosphoramidite chemical synthesis. Alternatively, RNA molecules may be generated by in vitro and in vivo transcription of DNA sequences encoding HsCENP-E. Such DNA sequences may be incorporated into a wide variety of vectors with suitable RNA polymerase promoters such as T7 or SP6. Alternatively, these cDNA constructs that synthesize complementary RNA, constitutively or inducibly, can be introduced into cell lines, cells, or tissues.

RNA molecules may be modified to increase intracellular stability and half-life. Possible modifications include, but are not limited to, the addition of flanking sequences at the 5′ and/or 3′ ends of the molecule, or the use of phosphorothioate or 2′ O-methyl rather than phosphodiesterase linkages within the backbone of the molecule. This concept is inherent in the production of PNAs and can be extended in all of these molecules by the inclusion of nontraditional bases such as inosine, queosine, and wybutosine, as well as acetyl-, methyl-, thio-, and similarly modified forms of adenine, cytidine, guanine, thymine, and uridine which are not as easily recognized by endogenous endonucleases.

Many methods for introducing vectors into cells or tissues are available and equally suitable for use in vivo, in vitro, and ex vivo. For ex vivo therapy, vectors may be introduced into stem cells taken from the patient and clonally propagated for autologous transplant back into that same patient. Delivery by transfection, by liposome injections, or by polycationic amino polymers may be achieved using methods which are well known in the art. (See, e.g., Goldman, C. K. et al. (1997) Nature Biotechnology 15: 462-466).

Any of the therapeutic methods described above may be applied to any subject in need of such therapy, including, for example, mammals such as dogs, cats, cows, horses, rabbits, monkeys, and most preferably, humans.

Alternatively, the target cell can be engineered to express a double stranded RNA molecule (dsRNA) and its effect on endogenous RNA in the host cells assessed via conventional means. Techniques for introducing dsRNA are known to one skilled in the art. See Fire A. (1999) Trends Genet. 15: 358-363; Sharp P A (1999) Genes Dev 13: 139-141; and Hunter C. (1999) Curr. Biol. 9: R440-R442. Thus, the dsRNA approach described above (e.g., RNAi) will prove effective in inactivating a cloned gene as well as in establishing gene expression profiling etc. See Fire et al. (1998) Nature 391: 80681 (1); and Montgomery et al. (1998) PNAS 95: 15502-15507). In an alternative embodiment, method(s) of the invention may be used to introduce a DNA fragment, which is anti-sense to an mRNA encoding a receptor for a drug. It is believed that the anti-sense heterologous DNA, will effectively decrease the expression of the drug receptor protein, thereby causing a decrease in drug binding to cells containing the anti-sense DNA, thus enabling one to further characterize a potential target etc. Other uses will become apparent to one skilled in the art.

The invention also encompasses vectors in which the heterologous nucleic acids are cloned into the vector in reverse orientation, but operably linked to a regulatory sequence that permits transcription of antisense RNA. Thus, an antisense transcript can be produced to all, or to a portion, of a target nucleic acid including both coding and non-coding regions.

In yet another embodiment, a heterologous or foreign nucleic acid molecule encoding a protein of SEQ ID NO:2 or a fragment thereof for use in the methods of the invention can be modified at the base moiety, sugar moiety or phosphate backbone to improve, e.g., the stability, hybridization, or solubility of the molecule. For example, the deoxyribose phosphate backbone of the nucleic acid molecules can be modified to generate peptide nucleic acids (see Hyrup B. et al. (1996) Bioorganic & Medicinal Chemistry 4 (1): 5-23).

As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup B. et al. (1996) supra; Perry-O'Keefe et al. Proc. Natl. Acad. Sci. 93: 14670-675.

Accordingly, PNAs of a target nucleic acid molecule can be used in therapeutic and diagnostic applications. For example, PNAs can be used as antisense agents for sequence-specific modulation of gene expression by, for example, inducing transcription or translation arrest or inhibiting replication.

V. Pharmaceutical Formulations and Modes of Administration

Pharmaceutically useful compositions comprising nucleic acid molecules encoding the novel HsCENP-E of the invention, antisense sequences thereto, polypeptides of SEQ ID NO:2 or fragments thereof having at least one HsCENP-E activity, antibodies to the herein disclosed protein and mimetics, agonists, antagonists, or inhibitors of HsCENP-E may be formulated according to known methods such as by the admixture of a pharmaceutically acceptable carrier. Examples of such carriers and methods of formulation may be found in Remington's Pharmaceutical Sciences (Maack Publishing Co, Easton, Pa.). To form a pharmaceutically acceptable composition suitable for effective administration, such compositions will contain an effective amount of the protein, DNA, RNA, or modulator as described herein.

A therapeutically effective dose refers to compositions of the invention that are administered to an individual in amounts sufficient to treat or diagnose human CENP-E mediated disorder. The effective amount may vary according to a variety of factors such as the individual's condition, weight, sex and age. Other factors include the mode of administration. An effective but non-toxic amount of the compound desired can be employed as a human CENP-E modulating agent.

Compounds identified according to the methods disclosed herein may be used alone at appropriate dosages defined by routine testing in order to obtain optimal modulation of a CENP-E polypeptide, or its activity while minimizing any potential toxicity. In addition, co-administration or sequential administration of other agents may be desirable.

Toxicity and therapeutic efficacy of such compounds can be determined by standard pharmaceutical procedures in cell cultures or experimental animals, e.g., for determining the LD₅₀. (the dose lethal to 50% of the population) and the ED₅₀ (the dose therapeutically effective in 50% of the population). The dose ratio between toxic and therapeutic effects is the therapeutic index and it can be expressed as the ratio ID₅₀/ED₅₀. Compounds which exhibit large therapeutic indices are preferred. The data obtained from these cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized.

The exact formulation, route of administration and dosage can be chosen by the individual physician in view of the patient's condition. See e.g. Fingl et al., in The Pharmacological Basis of Therapeutics, 1975, Ch. 1 p. 1. It should be noted that the attending physician would know how to and when to terminate, interrupt, or adjust administration due to toxicity, or to organ dysfunction. Conversely, the attending physician would also know to adjust treatment to higher levels if the clinical response were not adequate (precluding toxicity). The magnitude of an administrated dose in the management of the disorder of interest will vary with the severity of the condition to be treated and to the route of administration. The severity of the condition may, for example, be evaluated, in part, by standard prognostic evaluation methods. Further, the dose and perhaps dose frequency, will also vary according to the age, body weight, and response of the individual patient. A program comparable to that discussed above may be used in veterinary medicine. The dosage should not be so large as to cause adverse side effects, such as unwanted cross-reactions, anaphylactic reactions, and the like.

Generally, the dosage will vary with the age, condition, sex and extent of disease in the patient, counter indications, if any, and other such variables, to be adjusted by the individual physician. Dosage can vary from 0.001 mg/kg to 50 mg/kg, preferably 0.1 mg/kg to 1.0 mg/kg, of the agonist or antagonist of the invention, in one or more administrations daily, for one or several days.

For oral administration, the compositions are preferably provided in the form of scored or unscored tablets containing 0.01, 0.05, 0.1, 0.5, 1.0, 2.5, 5.0, 10.0, 15.0, 25.0, and 50.0 milligrams of the active ingredient for the symptomatic adjustment of the dosage to the patient to be treated. An effective amount of the drug is ordinarily supplied at a dosage level of from about 0.0001 mg/kg to about 100 mg/kg of body weight per day. The range is more particularly from about 0.001 mg/kg to 10 mg/kg of body weight per day. Even more particularly, the range varies from about 0.05 to about 1 mg/kg. Of course the dosage level will vary depending upon the potency of the particular compound. Certain compounds will be more potent than others. In addition, the dosage level will vary depending upon the bioavalability of the compound. The more bioavailable and potent the compound, the less compound will need to be administered through any delivery route, including but not limited to oral delivery. The dosages of HsCENP-E are adjusted when combined to achieve desired effects. On the other hand, dosages of these various agents may be independently optimized and combined to achieve a synergistic result wherein the pathology is reduced more than it would be if either agent were used alone. Those skilled in the art will employ different formulations for nucleotides than for proteins or their inhibitors. Similarly, delivery of polynucleotides or polypeptides will be specific to particular cells and conditions.

For example, a dose can be formulated in animal models to achieve a circulating plasma concentration range that includes the IC₅₀ as determined in cell culture (i.e., the concentration of the test compound which achieves a half-maximal disruption of the polypeptide complex, or a half-maximal inhibition of the cellular level and/or activity of a complex component). Such information can be used to more accurately determine useful doses in humans. Levels in plasma may be measured, for example, by HPLC.

The pharmaceutical compositions may be provided to the individual by a variety of routes such as subcutaneous, topical, oral and intramuscular. Administration of pharmaceutical compositions is accomplished orally or parenterally. Methods of parenteral delivery include topical, intra-arterial (directly to the tissue), intramuscular, subcutaneous, intramedullary, intrathecal, intraventricular, intravenous, intraperitoneal, or intranasal administration. The present invention also has the objective of providing suitable topical, oral, systemic and parenteral pharmaceutical formulations for use in the novel methods of treatment of the present invention. The compositions containing compounds identified according to this invention as the active ingredient for use in the modulation of HsCENP-E can be administered in a wide variety of therapeutic dosage forms in conventional vehicles for administration. For example, the compounds can be administered in such oral dosage forms as tablets, capsules (each including timed release and sustained release formulations), pills, powders, granules, elixirs, tinctures, solutions, suspensions, syrups and emulsions, or by injection. Likewise, they may also be administered in intravenous (both bolus and infusion), intraperitoneal, subcutaneous, topical with or without occlusion, or intramuscular form, all using forms well known to those of ordinary skill in the pharmaceutical arts.

For injection, the agents of the invention may be formulated in aqueous solutions, preferably in physiologically compatible buffers such as Hanks's solution, Ringer's solution, or physiological saline buffer. For such transmucosal administration, penetrants appropriate to the barrier to be permeated are used in the formulation. Such penetrants are generally known in the art.

Use of pharmaceutically acceptable carriers to formulate the compounds herein disclosed for the practice of the invention into dosages suitable for systemic administration is within the scope of the invention. With proper choice of carrier and suitable manufacturing practice, the compositions of the present invention, in particular, those formulated as solutions, may be administered parenterally, such as by intravenous injection. The compounds can be formulated readily using pharmaceutically acceptable carriers well known in the art into dosages suitable for oral administration. Such carriers enable the compounds of the invention to be formulated as tablets, pills, capsules, liquids, gels, syrups, slurries, suspensions and the like, for oral ingestion by a patient to be treated.

Agents intended to be administered intracellularly may be administered using techniques well known to those of ordinary skill in the art. For example, such agents may be encapsulated into liposomes, then administered as described above. Liposomes are spherical lipid bilayers with aqueous interiors. All molecules present in an aqueous solution at the time of liposome formation are incorporated into the aqueous interior. The liposomal contents are both protected from the external microenvironment and, because liposomes fuse with cell membranes, are efficiently delivered into the cell cytoplasm. Additionally, due to their hydrophobicity, small organic molecules may be directly administered intracellularly.

In addition to the active ingredients, these pharmaceutical compositions may contain suitable pharmaceutically acceptable carriers comprising excipients and auxiliaries which facilitate processing of the active compounds into preparations which can be used pharmaceutically. The preparations formulated for oral administration may be in the form of tablets, capsules, or solutions. The pharmaceutical compositions of the present invention may be manufactured in a manner that is itself known, e.g., by means of conventional mixing, dissolving, granulating, levitating, emulsifying, encapsulating, entrapping or lyophilizing processes.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran, optionally, the suspension may also contain suitable stabilizers or agents which increase the solubility of the compounds to allow for the preparation of highly concentrated solutions.

Pharmaceutical preparations for oral use can be obtained by combining the active compounds with solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

The examples below are provided to illustrate the subject invention and are not included for the purpose of limiting the invention.

### EXAMPLES

I. cDNA Library Construction and/or Isolation of cDNA Clones

MRC-5 human diploid lung fibroblast polyA RNA was obtained from a commercial source (Ambion) and used for RT-PCR mediated isolation of an HsCENP-E motor domain cDNA fragment. For amplification of a long motor domain fragment corresponding to amino acids 1 through 465 of the HsCENP-E motor domain, the following RT-PCR primers were employed:

5′ amplification primer:
5′ GCCCATGGCGGAGGAAGGAGCCGT 3′

In addition to HsCENP-E nucleotide sequence, this primer contains a GC-clamp followed by an NcoI restriction site nested around the ATG start codon for ease of subcloning into an expression vector.

3′ amplification primer:
5′ GCGGTACCGACAGATTCATCAATTTCTCG 3′

In addition to HsCENP-E nucleotide sequence, this primer contains a GC-clamp and KpnI restriction site for ease of subcloning into an expression vector.

The Titan One Tube RT-PCR System (Roche) was used according to manufacturer's instructions to reverse transcribe and amplify cDNA. Briefly, the reverse transcriptase reaction was carried out on 200 ng MRC-5 polyA RNA at 50° C. for 30 minutes. Prior to this reaction, the RNA template and primers were heated in water to 95° C. for 1 minute followed by immediate cooling on ice to denature any secondary structure at the 5′ end of the RNA template.

Following reverse transcription to cDNA, samples were subject to PCR amplification in a GeneAmp PCR System 9700 thermal cycler (PE Applied Biosystems). Samples were initially denatured at 94° C. for 2 minutes followed by 30 rounds of temperature cycling at 94° C. for 15 seconds, 52° C. for 30 seconds, and 68° C. for 2 minutes. A final 5-minute extension was carried out at 68° C. prior to cooling samples to 4° C. for storage.

Following PCR amplification of the HsCENP-E cDNA fragment, samples were purified from excess primer and nucleotides and digested with NcoI and KpnI restriction enzymes. The motor domain fragment was subcloned into the pTrcHis2C vector from Invitrogen.

The corresponding plasmid was called pTrcHis2C/HsCENP-E465.

For functional analysis of a smaller motor domain fragment of HsCENP-E corresponding to amino acids 1 through 340, the Expand High Fidelity PCR System (Roche) was used according to manufacturer's instructions to amplify a 1020 base pair fragment of the HsCENP-E motor domain from plasmid DNA (pTrcHis2C/HsCENP-E465).

The following amplification primers were used:

5′ amplification primer:
5′ GCCCATGGCGGAGGAAGGAGCCGT 3′

This primer is identical to the 5′ amplification primer described above.

3′ amplification primer:
5′ GCGTCGACAGTTGATACCTCATTAAC 3′

In addition to HsCENP-E nucleotide sequence, this primer contains a GC-clamp and SalI restriction site for ease of subcloning into an expression vector. Briefly, the following amplification conditions were used: 5 ng of plasmid template DNA was amplified by an initial denaturation of the PCR reaction at 94° C. for 2 minutes followed by 25 rounds of thermal cycling at 94° C. for 15 seconds, 55° C. for 30 seconds, and 72° C. for 1 minute. A final 5-minute extension was carried out at 72° C. prior to cooling samples to 4° C. for storage. PCR amplification was carried out in a GeneAmp PCR System 9700 thermal cycler (PE Applied Biosystems).

Following PCR, amplified material was purified from excess primers and nucleotides and subject to digestion with NcoI and SalI restriction enzymes. The HsCENP-E motor domain fragment corresponding to amino acids 1 through 340 was subcloned into the pET23D bacterial expression vector from Novagen.

The resultant plasmid is referred to as pET23D/HsCENP-E340. Insertion of the HsCENP-E cDNA in frame to a histidine linker present in the pET23D vector creates a series of 6 histidine residues (6×His Tag) at the C-terminus of the expressed protein. This 6×His Tag is useful for purification and immunological detection of the expressed protein.

Plasmid DNA from individual bacterial transformants was subject to sequence analysis to verify that no PCR-mediated sequence errors were present.

II. Sequencing and Analysis

The aforementioned plasmid DNA was subjected to sequencing analysis. Both coding and noncoding strands of the cloned HsCENP-E motor domains were sequence verified. Alignment versus the published nucleotide sequence of HsCENP-E (Genbank entry Z15005) or the translated amino acid sequence of HsCENP-E (NCBI Entrez Protein [www.ncbi.nlm.nih.gov/entrez/query.fcgi?db=Protein] entries: CAA78727, S28261, NP 001804, Q02224, 1819485A) was carried out using the GCG alignment package. In each instance, the nucleotide and amino acid sequence of the HsCENP-E motor domain fragments described herein was consistent with there being an Alanine residue at amino acid position 300 rather than a Proline residue as described in the entries listed above.

III. Site-Directed Mutagenesis of Isolated cDNA Clone

To investigate the functional significance of an alanine residue in HsCENP-E at amino acid position 300 compared to a proline residue at that position, site-directed mutagenesis was performed on the pET23D/HsCENP-E340 expression plasmid to change the nucleotide coding sequence to one specifying a proline at amino acid 300 of the HsCENP-E motor domain.

The QuikChange Site-Directed Mutagenesis Kit from Stratagene was used according to manufacturer's instructions. Briefly, PCR amplification of 5 ng of pET23D/HsCENP-E340 plasmid DNA was carried out in the presence of 125 ng each of two mutagenic nucleotide primers that contained the desired nucleotide change and that annealed to the same sequence on opposite strands of the plasmid. The following mutagenic oligonucleotides were used:

Mut35A:
5′ CCTTGGGAGGAAATCCAAAGACACGTATTATCTGC 3′
Mut35B:
5′ GCAGATAATACGTGTCTTTGGATTTCCTCCCAAGG 3′

The plasmid DNA was initially denatured at 95° C. for 30 seconds followed by 12 rounds of thermal cycling at 95° C. for 30 seconds, 55° C. for 1 minute, and 68° C. for 9 minutes in a GeneAmp PCR System 9700 thermal cycler (PE Applied Biosystems).

Following cooling of reactions to 4° C., 10 units of DpnI restriction enzyme was added to each 50 μL PCR reaction, mixed, and incubated at 37° C. for 1 hour to digest parental (non-mutated) supercoiled double-stranded DNA. One microliter of DpnI-treated DNA was transformed into XL-1 Blue Supercompetent cells according to manufacturer's instructions. Cells were spread on LB-ampicillin agar plates and incubated at 37° C. for more than 16 hours.

Individual bacterial transformant colonies were grown in liquid selective media at 37° C. and plasmid miniprep DNA was isolated from cells using the Wizard Plus SV Miniprep DNA Purification System from Promega. Samples were then subject to sequence analysis to confirm the presence of the desired nucleotide change.

To ensure that only the desired mutation was incorporated into the pET23D/HsCENP-E340 expression vector and that no other PCR-induced errors were present, sequence-verified expression vector containing the correct nucleotide change was digested with HpaI and SalI restriction enzymes and a 158 base pair fragment carrying the mutation of interest was isolated. This fragment was reintroduced into pET23D/HsCENP-E340 plasmid that had not been subject to prior PCR amplification. The resultant plasmid expression vector, pET23D/HsCENP-E340 A300P was transformed into BL21(DE3)pLysS cells for protein expression under induction condition “A” as described in Section VIII, Expression of HsCENP-E, below.

IV. Northern Analysis

Northern analysis is a laboratory technique used to detect the presence of a transcript of a gene and involves the hybridization of a labeled nucleotide sequence to a membrane on which RNAs from a particular cell type or tissue have been bound (See, e.g., Sambrook, supra ch. 7; Ausubel, 1995, supra, ch. 4 and 16).

V. Labeling and Use of Individual Hybridization Probes

Hybridization probes derived from SEQ ID NO:1 are employed to screen cDNAs, genomic DNAs, or mRNAs. Although the labeling of polynucleotides, consisting of about 20 base pairs, is specifically described, essentially the same procedure is used with larger nucleotide fragments. Polynucleotides are designed using state-of-the-art software such as OLIGO 4.06 software (National Biosciences) and labeled by Combining suitable amounts of an oligomer, e.g., 50 pmol of each oligomer, 250 μCi of ³²P-adenosine triphosphate (Amersham Pharmacia Biotech), and T4 polynucleotide kinase (DuPont NEN, Boston Mass.). The labeled polynucleotides are substantially purified using a SEPHADEX G-25 superfine size exclusion dextran bead column (Amersham Pharmacia Biotech). An aliquot containing 10⁷ counts per minute of the labeled probe is used in a typical membrane-based hybridization analysis of human genoniic DNA digested with one of the following endonucleases: Ase I, Bgl II, Eco RI, Pst I, XbaI, or Pvu II (DuPont NEN).

The DNA from each digest is fractionated on a 0.7% agarose gel and transferred to nylon membranes (Nytran Plus, Schleicher & Schuell, Durham N.H.). Hybridization is carried out for 16 hours at 40° C. To remove nonspecific signals, blots are sequentially washed at room temperature under increasingly stringent conditions up to 0.1×saline sodium citrate and 0.5% sodium dodecyl sulfate. After XOMAT-AR film (Eastman Kodak, Rochester N.Y.) is exposed to the blots to film for several hours, hybridization patterns are compared visually.

VI. Microarrays

In general, a chemical coupling procedure and an ink jet device can be used to synthesize array elements on the surface of a substrate (See, e.g., Baldeschweiler, supra). An array analogous to a dot or slot blot may also be used to arrange and link elements to the surface of a substrate using thermal, UV, chemical, or mechanical bonding procedures. A typical array may be produced by hand or using available methods and machines and contain any appropriate number of elements. After hybridization, nonhybridized probes are removed and a scanner used to determine the levels and patterns of fluorescence. The degree of complementarity and the relative abundance of each probe which hybridizes to an element on the microarray may be assessed through analysis of the scanned images.

Full-length cDNAs, Expressed Sequence Tags (ESTs), or fragments thereof may comprise the elements of the microarray. Fragments suitable for hybridization can be selected using software well known in the art such as LASERGENE software (DNASTAR). Full-length cDNAs, ESTs, or fragments thereof corresponding to one of the nucleotide sequences of the present invention, or selected at random from a cDNA library relevant to the present invention, are arranged on an appropriate substrate, e.g., a glass slide. The cDNA is fixed to the slide using, e.g., UV cross-linking followed by thermal and chemical treatments and subsequent drying (See. e.g., Schena, M. et al. (1995) Science 270: 467-470; Shalon, D. et al. (1996) Genome Res. 6: 639-645). Fluorescent probes are prepared and used for hybridization to the elements on the substrate. The substrate is analyzed by procedures described above.

VII. Complementary Polynucleotides

Sequences complementary to the HsCENP-E-encoding sequences, or any parts thereof, are used to detect, decrease, or inhibit expression of naturally occurring HsCENP-E. Although use of polynucleotides comprising from about 15 to 30 base pairs is described, essentially the same procedure is used with smaller or with larger sequence fragments. Appropriate polynucleotides are designed using OLIGO 4.06 software (National Biosciences) and the coding sequence of HsCENP-E. To inhibit transcription, a complementary oligonucleotide is designed from the most unique 5′ sequence and used to prevent promoter binding to the coding sequence. To inhibit translation, a complementary oligonucleotide is designed to prevent ribosomal binding to the HsCENP-E-encoding transcript.

VIII. Expression of HsCENP-E

Pilot expression studies were performed with the pET23D/HsCENP-E340 construct to determine optimal conditions for expression of the 340 amino acid HsCENP-E motor domain in bacteria. Briefly, the pET23D/HsCENP-E340 expression construct was tested in both BL21(DE3) and BL21(DE3)pLysS bacterial strains from Novagen under three different induction conditions described here as conditions “A”, “B”, AND “C.”

For induction condition A, bacterial transformants carrying the expression plasmid were grown in liquid selective media at 37° C. with continuous shaking to an O.D.600 of 0.4. Once the proper optical density was reached, IPTG was added to the bacterial cultures to a final concentration of 100 μM and induction of HsCENP-E340 expression was allowed to proceed for 18-20 hours at 25° C. with continuous shaking.

For induction condition B, bacterial transformants carrying the pET23D/HsCENP-E340 expression plasmid were grown in liquid selective media at 37° C. with continuous shaking to an O.D.600 of 0.6 to 1.0. IPTG was then added to the cultures to a final concentration of 0.5 mM and induction of HsCENP-E340 protein expression was allowed to proceed for 4 hours at 25° C. with continued shaking.

For induction condition C, bacterial transformants carrying the pET23D/HsCENP-E340 expression plasmid were grown in liquid selective media at 37° C. with continuous shaking to an O.D.600 of 0.6. IPTG was then added to bacterial cultures to a final concentration of 0.4 mM to induce HsCENP-E340 protein expression for 3 hours at 37° C.

Bacterial samples pre- and post-addition of IPTG were collected from each induction condition and expression of HsCENP-E340 protein was assessed by Western analysis using a 6×His Tag antibody (peroxidase conjugated Monoclonal Anti-polyhistidine antibody, Clone HIS-1, from Sigma) for immunodetection. Briefly, bacterial cell pellets were extracted with B-PER Bacterial Protein Extraction Reagent from Pierce. Insoluble material was pelleted by centrifugation and the soluble extract was transferred to a new tube. The insoluble material was further extracted with RIPA buffer (1% NP40, 0.5% sodium deoxycholate, 0.1% SDS in PBS with protease inhibitor tablets [Complete Mini, EDTA free protease inhibitor cocktail tablets from Roche] added fresh) and used for analysis.

Total protein concentration from extracts of soluble and insoluble material was determined by using Bio-Rad D_{c} Protein Assay Reagents (Bio-Rad Laboratories) according to manufacturer's instructions. Equal amounts of protein from each sample were analyzed by Western to determine which induction condition was best for expressing high levels of soluble, biologically active HsCENP-E340 protein. It was determined that use of bacterial strain BL21(DE3)pLysS under induction condition A provided the best and most convenient means for expressing soluble, biologically active HsCENP-E340 protein in quantity.

Methods for protein expression and purification are discussed in Ausubel (1995, supra, ch 10 and 16). Purified HsCENP-E obtained by these methods can be used directly in the following activity assay.

IX. Demonstration of HsCENP-E Activity

A microtubule motility assay for HsCENP-E activity measures motor domain function. In this assay, recombinant HsCENP-E is immobilized onto a glass slide or similar substrate. Taxol-stabilized bovine brain microtubules (commercially available) in a solution containing ATP and cytosolic extract are perfused onto the slide. Movement of microtubules as driven by HsCENP-E motor activity can be visualized and quantified using video-enhanced light microscopy and image analysis techniques. HsCENP-E activity is directly proportional to the frequency and velocity of microtubule movement.

X. Functional Assays

HsCENP-E function is assessed by expressing the sequences encoding HsCENP-E at physiologically elevated levels in mammalian cell culture systems. cDNA is subcloned into a mammalian expression vector containing a strong promoter that drives high levels of cDNA expression. Vectors of choice include pCMV SPORT (Life Technologies) and pCR3.1 (Invitrogen, Carlsbad Calif.), both of which contain the cytomegalovirus promoter. 5-10 μg of recombinant vector are transiently transfected into a human cell line, preferably of endothelial or hematopoietic origin, using either liposome formulations or electroporation. 1-2 μg of an additional plasmid containing sequences encoding a marker protein are co-transfected. Expression of a marker protein provides a means to distinguish transfected cells from nontransfected cells and is a reliable predictor of cDNA expression from the recombinant vector. Marker proteins for use include any one of the commercially available markers e.g., Green Fluorescent Protein (GFP; Clontech), CD64, or a CD64-GFP fusion protein. Flow cytometry (FCM), an automated, laser optics-based technique, is used to identify transfected cells expressing GFP or CD64-GFP, and to evaluate cellular properties, for example, their apoptotic state. FCM detects and quantifies the uptake of fluorescent molecules that diagnose events preceding or coincident with cell death. These events include changes in nuclear DNA content as measured by staining of DNA with propidium iodide; changes in cell size and granularity as measured by forward light scatter and 90 degree side light scatter, down-regulation of DNA synthesis as measured by decrease in bromodeoxyuridine uptake; alterations in expression of cell surface and intracellular proteins as measured by reactivity with specific antibodies; and alterations in plasma membrane composition as measured by the binding of fluorescein-conjugated Annexin V protein to the cell surface. Methods in flow cytometry are discussed in Ormerod, M. G. (1994) Flow Cytometry, Oxford, New York N.Y.

The influence of HsCENP-E on gene expression can be assessed using highly purified populations of cells transfected with sequences encoding HsCENP-E and either CD64 or CD64-GFP. CD64 and CD64-GFP are expressed on the surface of transfected cells and bind to conserved regions of human immunoglobulin G (IgG). Transfected cells are efficiently separated from nontransfected cells using magnetic beads coated with either human IgG or antibody against CD64 (DYNAL, Lake Success N.Y.). mRNA can be purified from the cells using methods well known by those of skill in the art. Expression of mRNA encoding HsCENP-E and other genes of interest can be analyzed by northern analysis or microarray techniques.

XI. Production of HsCENP-E Specific Antibodies

HsCENP-E substantially purified using polyacrylamide gel electrophoresis (PAGE; see, e.g., Harrington, M. G. (1990) Methods Enzymol. 182: 488-495), or other purification techniques, is used to immunize rabbits and to produce antibodies using standard protocols.

Alternatively, the HsCENP-E amino acid sequence is analyzed using LASERGENE software (DNASTAR) to determine regions of high immunogenicity, and a corresponding oligopeptide is synthesized and used to raise antibodies by means known to those of skill in the art. Methods for selection of appropriate epitopes, such as those near the C-terminus or in hydrophilic regions are well described in the art (See, e.g., Ausubel, 1995, supra, ch. 11).

Typically, oligopeptides 15 residues in length are synthesized using an ABI 431A peptide synthesizer (Perkin-Elmer) using fmoc-chemistry and coupled to KLH (Sigma-Aldrich, St. Louis Mo.) by reaction with N-maleimidobenzoyl-N-hydroxysuccinimide ester (MBS) to increase immunogenicity (See, e.g., Ausubel, 1995, supra). Rabbits are immunized with the oligopeptide-KLH complex in complete Freund's adjuvant. Resulting antisera are tested for antipeptide activity by, for example, binding the peptide to plastic, blocking with 1% BSA, reacting with rabbit antisera, washing, and reacting with radio-iodinated goat anti-rabbit IgG.

XII. Purification of Naturally Occurring HsCENP-E Using Specific Antibodies

Naturally occurring or recombinant HsCENP-E are substantially purified by immunoaffinity chromatography using antibodies specific for HsCENP-E. An immunoaffinity column is constructed by covalently coupling anti-HsCENP-E antibody to an activated chromatographic resin, such as CNBr-activated SEPHAROSE (Amersham Pharmacia Bictech). After the coupling, the resin is blocked and washed according to the manufacturer's instructions.

Media containing HsCENP-E are passed over the immunoaffinity column, and the column is washed under conditions that allow the preferential absorbance of HsCENP-E (e.g., high ionic strength buffers in the presence of detergent). The column is eluted under conditions that disrupt antibody/HsCENP-E binding (e.g., a buffer of pH 2 to pH 3, or a high concentration of a chaotrope, such as urea or thiocyanate ion), and HsCENP-E is collected.

XIII. Identification of Agents which Interact with HsCENP-E

HsCENP-E, or biologically active fragments thereof, are labeled with ¹²⁵I Bolton-Hunter reagent (See, e.g., Bolton et al. (1973) Biochem. J. 133: 529). Candidate molecules previously arrayed in the wells of a multi-well plate are incubated with the labeled HsCENP-E, washed, and any wells with labeled HsCENP-E complex are assayed. Data obtained using different concentrations of HsCENP-E are used to calculate values for the number, affinity, and association of HsCENP-E with the candidate molecules.

Various modifications and variations of the described methods and systems of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

### SUMMARY OF SEQUENCES

SEQ ID NO:1 discloses a nucleotide sequence of 1395 base pairs that encodes a novel motor domain of human CENP-E protein—referred to as CENP-E 465. The remainder of the sequence (bp 1396-1488) is a polylinker derived from the pTrcHis2C expression vector.

SEQ ID NO:2 is the amino acid sequence of the motor protein encoded by the nucleotide sequence as set forth in SEQ ID NO:1-465 amino acids. Note that amino acids 466-496 are not derived from the CENP-E motor domain, rather it is a linker region plus a myc epitope and 6×His Tag provided by the pTrcHis2C expression vector.

SEQ ID NO:3 corresponds to the nucleotide sequence of 1020 base pairs which encodes a novel motor domain of human CENP-E protein of 340 amino acids, also referred to as CENP-E340. The remainder of the sequence (bp 1021-1065) is a polylinker derived from the pET23D expression vector. See the examples for further details of this expression vector.

SEQ ID NO:4 is the amino acid sequence of the motor protein encoded by the nucleotide sequence as set forth in SEQ ID NO: 3-340 amino acids. Note that amino acids 341-355 are not derived from the CENP-E motor domain, rather it is a linker region plus a 6×His Tag provided by the pET23D expression vector.

1. A purified polypeptide comprising an amino acid sequence selected from the group consisting of:
a) an amino acid sequence as set forth in SEQ ID NO:2, and
b) an amino acid sequence comprising amino acid residue 1 to amino acid residue 340 of SEQ ID NO:2.

2. (canceled)

3. A pharmaceutical composition comprising the polypeptide of claim 1 and a pharmaceutically acceptable excipient.

4. A composition of claim 3, wherein the polypeptide has the sequence of SEQ ID NO:2.

5. A method for screening a compound for effectiveness as an agonist of the polypeptide of claim 1, comprising:
a) exposing a sample comprising the polypeptide of claim 1 to a compound, and
b) detecting agonist activity in the sample.

6. A method for screening a compound for effectiveness as an antagonist of the polypeptide of claim 1, the method comprising:
a) exposing a sample comprising a polypeptide of claim 1 to a compound, and
b) detecting antagonist activity in the sample.

7. An isolated nucleic acid molecule comprising a sequence of nucleotides as set forth in SEQ ID NO:1.

8. An isolated polynucleotide which hybridizes under conditions of 250 mM NaCl, 25 mM trisodium citrate, 1% SDS, 50% formamide and 200 μg/ml ssDNA at 42° C., and wash conditions of 15 mM NaCl, 1.5 mM trisodium citrate, and 0.1% SDS at 68° C. to the sequence of nucleotides as set forth in claim 7.

9. A method for detecting a nucleic acid molecule having a sequence of nucleotides substantially similar to the nucleic acid molecule of claim 7, the method comprising the steps of:
a) hybridizing the nucleic acid molecule of claim 7 to at least one nucleic acid in a sample, under conditions favoring the formation of a hybridization complex; and
b) detecting the hybridization complex, wherein said hybridization is performed at 42° C. in a solution containing 250 mM NaCl, 25 mM trisodium citrate, 1% SDS, 50% formamide and 200 μg/ml ssDNA followed by washing at 68° C. in a solution of 15 mM NaCl, 1.5 mM trisodium citrate, and 0.1% SDS wherein the presence of the hybridization complex correlates with the presence of the polynucleotide in the sample.

10. The method of claim 9 further comprising amplifying the polynucleotide prior to hybridization.

11. An isolated nucleic acid molecule comprisisng a sequence of nucleotides that encode a polypeptide comprising the amino acid sequence as set forth in SEQ ID NO: 2.

12. An expression vector comprising the nucleic acid molecule of claim 7.

13. A host cell comprising the expression vector of claim 12.

14. A method for producing the polypeptide of claim 1, comprising the steps of:
a) culturing the host cell of claim 13 under conditions suitable for the expression of the polypeptide; and
b) recovering the polypeptide from the host cell culture.

15. A method of modulating cellular proliferation in a mammal in need thereof comprising administering to said mammal an amount of the pharmaceutical composition of claim 3 effective to modulate cellular proliferation, said composition comprising a pharmaceutically acceptable vehicle and a HsCENP-E protein characterized as having an ATP binding site, and a motor domain comprising an amino acid sequence from amino acid at position 1 through amino acid at position 340 as set forth in SEQ ID NO:2.

16. A method for inhibiting HsCENP-E mediated/induced cellular proliferation of a cell in culture, said method comprising the steps of:
a) providing an oligonucleotide comprising at least 18 contiguous nucleotide bases which are complementary to a nucleotide base sequence region contained in a nucleic acid sequence as set forth in SEQ ID NO.1, and
b) contacting said cell with said oligonucleotide under conditions such that said oligonucleotide is delivered within said cell and hybridizes with said nucleotide base sequence region, thereby inhibiting HsCENP-E mediated/induced cellular proliferation of said cell.

17. A method of detecting the presence of cancer in an individual comprising:
(a) obtaining a biological sample from said individual;
(b) incubating said biological sample with at least one antibody which is immunoreactive with a gene product encoded by the nucleic acid molecule of claim 7;
(c) detecting immunoconjugates which form as a consequence of the incubation of step (b); and
(d) relating the amount of immunoconjugates of step (c) to the presence of cancer, wherein cancer is present when said amount is greater than a threshold value.

18. The substantially purified polypeptide of claim 1, wherein said polypeptide comprises an amino acid sequence as set forth in SEQ ID NO:2.

19. The substantially purified polypeptide of claim 1, wherein said polypeptide comprises an amino acid sequence comprising amino acids at position 1 through 340 of SEQ ID NO:2.
